(19) United States
(12) Patent Application Publication
(10) Pub. No.: US 2012/0190042 A1
(43) Pub. Date: Jul. 26, 2012

(54) PROTEIN BIOMARKERS AND THERAPEUTIC TARGETS FOR OSTEOARTHRITIS

(76) Inventors: Reuben Gobezie, Cleveland Heights, OH (US); David M. Lee, Basel (CH)

(21) Appl. No.: 13/145,796

(22) PCT Filed: Jan. 22, 2010
(86) PCT No.: PCT/US10/21741

§ 371 Date: Feb. 2, 2012

Related U.S. Application Data
(60) Provisional application No. 61/146,524, filed on Jan. 22, 2009.

(51) Int. Cl. G01N 33/566; G01N 33/573

(52) U.S. Cl. 435/7.4; 436/501

(57) ABSTRACT

The present invention relates to the identification and use of protein expression profiles with clinical relevance to osteoarthritis. In particular, the invention provides the identity of marker proteins whose expressions are correlated with OA, and/or OA progression. Methods and kits are described for using these protein expression profiles in the study and/or diagnosis of OA, in the determination of the degree of advancement of OA, and in the selection and/or monitoring of treatment regimens. The invention also relates to the screening of drugs that modulate expression of these proteins or nucleic acid molecules encoding these proteins, in particular for the development of disease-modifying OA agents.

### RELATED APPLICATION

This application claims priority from U.S. Provisional Application No. 61/146,524, filed Jan. 22, 2009, the subject matter, which is incorporated herein by reference.

### BACKGROUND

Musculoskeletal conditions affect hundreds of millions of people around the world and this figure is expected to increase sharply due to the predicted doubling of the population over 50 by the year 2020 (“The Global Burden of Disease. A Comprehensive Assessment of Mortality and Disability from Diseases, Injuries, and Risk Factors in 1990 and Projected to 2020”, C. J. L. Murray and A. D. Lopez (Eds.), 1996, Harvard University Press: Cambridge, Mass.). Musculoskeletal conditions give rise to enormous healthcare expenditures and loss of economic productivity, and therefore have a huge impact on society. In the U.S. alone, musculoskeletal conditions were estimated to have cost $214 billion in 1995 (A. Praemer et al., “Musculoskeletal Conditions in the United States”, 2nd Ed., 1999, American Academy of Orthopaedic Surgeons Rosemont, Ill.). While there are many types of musculoskeletal conditions, osteoarthritis is one of the most common chronic musculoskeletal disorders encountered by physicians throughout the world.

Osteoarthritis (OA) is a non-inflammatory joint disease, which is characterized by the breakdown of joint cartilage. It may affect one or more joints in the body, including those of the fingers, neck, shoulder, hips, knees, lower spine region, and feet. OA can cause pain and severely impair mobility and lower extremity function (E. Bagge et al., Age Ageing, 1992, 21: 160-167; D. Hamerman, Ann. Rheum. Dis., 1995, 54: 82-85; J. Jordan et al., J. Rheumatol., 1997, 24: 1344-1349; S. M. Ling and J. M. Bathon, J. Am. Geriatr. Soc., 1998, 46: 216-225), which can lead to disability and difficulty maintaining independence (A. A. Guccione et al., Am. J. Public Health, 1994, 84: 351-358; M. A Gignac et al., J. Gerontol. B: Psychol. Sci. Soc. Sci., 2000, 55: 362-372; M. C. Corti and C. Rignon, Aging Clin. Exp. Res., 2003, 15: 359-363).

Currently, diagnosis of OA is typically based upon radiological examination as well as clinical observations including localized tenderness, use-related pain, bony or soft tissue swelling, joint instability, limited joint function, muscle spasm, and crepitus (i.e., cracking or grinding sensation). While the diagnosis of OA is often suggested on physical examination, radiographic evaluation is generally used to confirm the diagnosis or assess the severity of the disease. The radiographic hallmarks of OA include nonuniform joint space loss, osteophyte formation, cyst formation, and subchondral sclerosis. While these characteristic features are generally present in X-ray images of “severe” or “late” OA, patients with “early” OA may not show radiographic evidence of bony changes, joint space narrowing and/or osteophytosis, making the diagnosis unclear or difficult to establish.

### SUMMARY OF THE INVENTION

The present invention relates to the use of protein expression profiles with clinical relevance to osteoarthritis. In particular, the invention provides the identity of proteins, whose expression is correlated with osteoarthritis (OA) and/or with different phases of advancement of the disease. These protein expression profiles may be applied to the diagnosis and staging of disease useful for prognostic purposes in OA. Compared to existing methods of diagnosis, the protein expression profiles disclosed herein constitute a more robust signature of OA and OA progression, and provide a more reliable basis for the selection of appropriate therapeutic regimens. The invention also relates to the screening of drugs that target these biomarkers, in particular for the development of new therapeutics for the treatment of OA.

In general, the invention involves the use of expression profiles of the marker proteins listed in attached Tables 1-6 for diagnosing osteoarthritis. More specifically, the present invention provides methods for distinguishing between stages of osteoarthritis. Methods are provided that comprise steps of: providing a biological sample obtained from a subject to be tested; determining, in the biological sample, the level of expression of a one or more of polypeptides selected from the group consisting of proteins presented in Tables 1-6.

In one embodiment, the proteins can comprise at least one of alpha-2-macroglobulin precursor, fibronectin precursor, Chain B structure of complement C3b, C9 complement protein, coagulation factor II precursor, alpha-1-antichymotrypsin, complement component C3, complement factor H, inter-alpha-trypsin heavy chain H1 precursor, complement factor H isoform a precursor, gelsolin isoform a precursor, ceruloplasmin, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In other embodiments, the proteins can comprise at least one of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), 5100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In still other embodiments, the proteins can comprise at least one of fibronectin precursor, complement component C3, alpha-2-macroglobulin precursor, Chain B Structure of Complement C3b, complement factor H, fibronectin 1 isoform 3 preproprotein, alpha-2-macroglobulin, collagen type IV alpha 1, alph 2 type IV collagen preprotein, inter-alpha-trypsin inhibitor, C-terminal inter-alpha trypsin inhibitor, ceruloplasmin, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, serum albumin, COMP, ALB protein, gelsolin isoform a precursor, complement factor B, fibulin-1 isoform D precursor, inter-alpha-globulin inhibitor H4, valosin-containing protein, Vitamin D-binding protein precursor, complement component 2 precursor, annexin A2, Annexin A2 isoform 2, homerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

In further embodiments, the proteins can comprise at least one of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, homerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

In still other embodiments, the proteins can comprise at least one of alpha-2-macroglobulin precursor, fibronectin precursor, Chain B structure of complement C3b, C9 complement protein, coagulation factor II precursor, alpha-1-antichymotrypsin, complement component C3, complement factor H, inter-alpha-trypsin heavy chain H1 precursor, complement factor H isoform a precursor, gelsolin isoform a precursor, ceruloplasmin, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, fibronectin 1 isoform 3 preproprotein, alpha-2-macroglobulin, collagen type IV alpha 1, C-terminal inter-alpha trypsin inhibitor, phosphatidylinositol-glycan-specific phospholipase D1, complement component 6 isoform CRA_b, Chain A Crystal Structure of human Galectin-7, serum albumin, ALB protein, inter-alpha-globulin inhibitor H4, valosin-containing protein, Vitamin D-binding protein precursor, complement component 2 precursor, annexin A2, homerin precursor, Protein S100-A7 (psoriasin), coagulation factor XIII, B Chain Alpha-Ferrous-Carbonmonoxy, insulin-like growth factor binding protein acid labile, histidine-rich glycoprotein precursor, phospholipid transfer protein isoform a precursor, coagulation factor XIII B chain, vitronectin, biotinidase precursor, alpha-1-B-glycoprotein, Coagulation factor XIII B chain precursor, vanin 1 precursor, extracellular matrix protein 1 isoform precursor, histidine-rich glycoprotein, dopamine beta-hydroxylase precursor, Chain A Crystal Structure of Native Heparin Cofactor Ii, inter-alpha (globulin) inhibitor H1, vitronectin precursor, Vitamin K-dependent protein S precursor, GRP78, analogs and fragments thereof; and any combination thereof.

In other embodiments, the proteins can comprise at least one of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), 5100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, fibronectin 1 isoform 3 preproprotein, collagen type IV alpha 1, C-terminal inter-alpha trypsin inhibitor, phosphatidylinositol-glycan-specific phospholipase D1, complement component 6 isoform CRA_b, Chain A Crystal Structure of human Galectin-7, ALB protein, valosin-containing protein, Vitamin D-binding protein precursor, complement component 2 precursor, annexin A2, homerin precursor, Protein S100-A7 (psoriasin), coagulation factor XIII, B Chain Alpha-Ferrous-Carbonmonoxy, insulin-like growth factor binding protein acid labile, histidine-rich glycoprotein precursor, phospholipid transfer protein isoform a precursor, coagulation factor XIII B chain, vitronectin, biotinidase precursor, alpha-1-B-glycoprotein, Coagulation factor XIII B chain precursor, vanin 1 precursor, extracellular matrix protein 1 isoform precursor, histidine-rich glycoprotein, dopamine beta-hydroxylase precursor, Chain A Crystal Structure of Native Heparin Cofactor Ii, inter-alpha (globulin) inhibitor H1, vitronectin precursor, Vitamin K-dependent protein S precursor, GRP78, analogs and fragments thereof; and any combination thereof.

In certain embodiments, providing an osteoarthritis diagnosis to the subject comprises comparing the test protein expression profile to a control protein expression, wherein the difference between the test protein expression profile and the control protein expression profile is indicative of early or late stage osteoarthritis in the subject; and based on the comparison, identifying osteoarthritis in the subject as early stage or late stage osteoarthritis.

In certain embodiments, the control protein expression profile is an early stage osteoarthritis expression profile, and the difference is indicative of late stage osteoarthritis. In such embodiments, the difference may be selected from the group consisting of an increase or decrease in the level of expression of one or more proteins selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In other embodiments, the control protein expression profile is a late stage osteoarthritis expression profile, and the difference is indicative of early stage osteoarthritis. In such embodiments, the difference may be selected from the group consisting of an increase or decrease in the level of expression of one or more proteins selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In other embodiments, the control protein expression profile is a healthy or normal expression profile, and the difference is indicative of early or late stage osteoarthritis. In such embodiments, the difference may be selected from the group consisting of an increase or decrease in the level of expression of one or more proteins selected from the group consisting of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, homerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

In still other embodiments, the control protein expression profile is a late or early stage osteoarthritis expression profile, and the difference is indicative of normal or healthy subject. In such embodiments, the difference may be selected from the group consisting of an increase or decrease in the level of expression of one or more proteins selected from the group consisting of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, hornerin precursor, complement component 1 subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

In these methods, the biological sample can comprise a sample of blood or blood product, a sample of urine, a sample of joint fluid, a sample of saliva, or a sample of synovial fluid. In certain preferred embodiments, the biological sample comprises a sample of synovial fluid. Determination of the level of expression of one or more of polypeptides according to the present invention may comprise exposing the biological sample to at least one antibody specific for at least one of said polypeptides.

In certain embodiments, the subject is a human being, for example, a patient suspected of having osteoarthritis or a patient diagnosed with osteoarthritis but whose stage of osteoarthritis is unknown.

The inventive methods may further comprise a step of selecting a therapy for the subject based on the determination of the stage of osteoarthritis for the subject.

In yet another aspect, the present invention provides OA expression profile maps comprising expression level information for one or more of polypeptides selected from the group consisting of the proteins presented in Tables 1-6, analogs and fragments thereof. The OA expression profile may comprise expression level information for at least one biological sample obtained from a healthy individual, an individual with early stage osteoarthritis or an individual with late osteopathic osteoarthritis.

In still another aspect, the present invention provides kits for identifying the stage of osteoarthritis in a subject. Inventive kits comprise at least one reagent that specifically detects expression levels of at least one biomarker selected from the group consisting of: polypeptides selected from the group consisting of the proteins presented in Tables 1 and 3, analogs and fragments thereof, and nucleic acid molecules comprising polynucleotide sequences coding for polypeptides selected from the group consisting of the proteins presented in Tables 1 and 3, analogs and fragments thereof, and instructions for using said kits for identifying osteoarthritis in a subject as early Osteopathic or late osteoarthritic osteoarthritis.

In certain embodiments, the reagent that specifically detects expression levels of at least one biomarker comprises an antibody that specifically binds to at least one the polypeptides. In other embodiments, the reagent comprises a nucleic acid probe complementary to a polynucleotide sequence coding for at least one of the polypeptides. For example, the nucleic acid probe may be a cDNA or an oligonucleotide, and, in certain embodiments, is immobilized on a substrate surface.

Kits of the present invention may further comprise instructions required by a regulatory agency (e.g., the United States Food and Drug Administration) for use in in vitro diagnostic products; one or more of: extraction buffer/reagents and protocol, amplification buffer/reagents and protocol, hybridization buffer/reagents and protocol, immunodetection buffer/reagents and protocol, and labeling buffer/reagents and protocol, and/or at least one OA expression profile map as described above.

These and other objects, advantages and features of the present invention will become apparent to those of ordinary skill in the art having read the following detailed description of the preferred embodiments.

### DETAILED DESCRIPTION

Throughout the specification, several terms are employed that are defined in the following paragraphs.

The term “subject” and “individual” are used herein interchangeably. They refer to a human or another mammal (e.g., primate, dog, cat, goat, horse, pig, mouse, rat, rabbit, and the like), that can be afflicted with osteoarthritis, but may or may not have the disease. In many embodiments, the subject is a human being.

The term “subject suspected of having osteoarthritis (OA)” refers to a subject that presents one or more symptoms indicative of OA (e.g., joint pain, localized tenderness, bony or soft tissue swelling, joint instability, crepitus) or that is being screened for OA (e.g., during a routine physical examination). A subject suspected of having OA may also have one or more risk factors (e.g., age, obesity, traumatic injury, overuse due to sports or occupational stresses, or family history). The term encompasses individuals who have not been tested for OA as well as individuals who have received an initial diagnosis (e.g., based on radiological examination) but for whom the stage of OA is not known.

The terms “osteoarthritis stage”, “osteoarthritis phase”, and “stage osteoarthritis” are used herein interchangeably and refer to the degree of advancement or progression of the disease. The present invention provides a means for determining the stage of the disease. In particular, the methods provided herein allows detection of “mild” or “early” stage OA, and of “severe” or “late” stage OA. Other staging systems known in the art include, for example, that developed by Marshall (W. Marshall, J. Rheumatol., 1996, 23: 582-584).

As used herein, the term “diagnosis” refers to a process aimed at determining if an individual is afflicted with a disease or ailment. In the context of the present invention, “diagnosis of OA” refers to a process aimed at one or more of: determining if an individual is afflicted with OA, and determining the stage of the disease (e.g., early OA or late OA).

The term “biological sample” is used herein in its broadest sense. A biological sample may be obtained from a subject (e.g., a human) or from components (e.g., tissues) of a subject. The sample may be of any biological tissue or fluid with which biomarkers of the present invention may be assayed. Frequently, the sample will be a “clinical sample”, i.e., a sample derived from a patient. Such samples include, but are not limited to, bodily fluids which may or may not contain cells, e.g., blood, urine, synovial fluid, saliva, and joint fluid; tissue or fine needle biopsy samples, such as from bone or cartilage; and archival samples with known diagnosis, treatment and/or outcome history. Biological samples may also include sections of tissues such as frozen sections taken from histological purposes. The term biological sample also encompasses any material derived by processing the biological sample. Derived materials include, but are not limited to, cells (or their progeny) isolated from the sample, proteins or nucleic acid molecules extracted from the sample. Processing of the biological sample may involve one or more of, filtration, distillation, extraction, concentration, inactivation of interfering components, addition of reagents, and the like.

The terms “normal” and “healthy” are used herein interchangeably. They refer to an individual or group of individuals who have not shown any OA symptoms, including joint pain, and have not been diagnosed with cartilage injury or OA. Preferably, said normal individual (or group of individuals) is not on medication affecting OA and has not been diagnosed with any other disease. In certain embodiments, normal individuals have similar sex, age, body mass index as compared with the individual from which the sample to be tested was obtained. The term “normal” is also used herein to qualify a sample isolated from a healthy individual.

In the context of the present invention, the term “control sample” refers to one or more biological samples isolated from an individual or group of individuals that are normal (i.e., healthy). A control sample can also refer to a biological sample isolated from a patient or group of patients diagnosed with a specific stage of OA (e.g., early OA or late OA). The term “control sample” (or “control”) can also refer to the compilation of data derived from samples of one or more individuals classified as normal, or one or more individuals diagnosed with OA, a specific stage of OA, or one or more individuals having undergone treatment of OA.

The terms “OA biomarker” and “biomarker” are used herein interchangeably. They refer to a protein selected from the set of proteins provided by the present invention and whose expression profile was found to be indicative of OA and/or a particular stage of OA. The term “biomarker” also encompasses nucleic acid molecules comprising a nucleotide sequence, which codes for a marker protein of the present invention as well as polynucleotides that hybridize with portions of these nucleic acid molecules.

As used herein, the term “indicative of OA”, when applied to a biomarker, refers to an expression pattern or profile which is diagnostic of OA or a stage of OA such that the expression pattern is found significantly more often in patients with the disease or a stage of the disease than in patients without the disease or another stage of the disease (as determined using routine statistical methods setting confidence levels at a minimum of 95%). Preferably, an expression pattern which is indicative of OA is found in at least 60% of patients who have the disease and is found in less than 10% of subjects who do not have the disease. More preferably, an expression pattern which is indicative of OA is found in at least 70%, at least 75%, at least 80%, at least 85%, at least 90%, at least 95% or more in patients who have the disease and is found in less than 10%, less than 8%, less than 5%, less than 2.5%, or less than 1% of subjects who do not have the disease.

As used herein, the term “differentially expressed biomarker” refers to a biomarker whose level of expression is different in a subject (or a population of subjects) afflicted with OA relative to its level of expression in a healthy or normal subject (or a population of healthy or normal subjects). The term also encompasses a biomarker whose level of expression is different at different stages of the disease (e.g., mild or early OA, severe or late OA). Differential expression includes quantitative, as well as qualitative, differences in the temporal or cellular expression pattern of the biomarker. As described in greater details below, a differentially expressed biomarker, alone or in combination with other differentially expressed biomarkers, is useful in a variety of different applications in diagnostic, staging, therapeutic, drug development and related areas. The expression patterns of the differentially expressed biomarkers disclosed herein can be described as a fingerprint or a signature of OA, OA stage and OA progression. They can be used as a point of reference to compare and characterize unknown samples and samples for which further information is sought. The term “decreased level of expression” as used herein, refers to a decrease in expression of at least 10% or more. For example, 20%, 30%, 40%, or 50%, 60%, 70%, 80%, 90% or more, or a decrease in expression of greater than 1-fold, 2-fold, 3-fold, 4-fold, 5-fold, 10-fold, 50-fold, 100-fold or more as measured by one or more methods described herein. The term “increased level of expression” as used herein, refers to an increase in expression of at least 10% or more. For example, 20%, 30%, 40%, or 50%, 60%, 70%, 80%, 90% or more or an increase in expression of greater than I-fold, 2-fold, 3-fold, 4-fold, 5-fold, 10-fold, 50-fold, 100-fold or more as measured by one or more methods, such as method described herein.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is the full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains, such as oxidation of sulfhydryl groups. Thus, the term “protein” (or its equivalent terms) is intended to include the amino acid sequence of the full-length native protein, subject to those modifications that do not change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

The term “protein analog”, as used herein, refers to a polypeptide that possesses a similar or identical function as the full-length native protein but need not necessarily comprise an amino acid sequence that is similar or identical to the amino acid sequence of the protein, or possesses a structure that is similar or identical to that of the protein. Preferably, in the context of the present invention, a protein analog has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the full-length native protein.

The term “protein fragment”, as used herein, refers to a polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of a marker protein may or may not possess a functional activity of the full-length native protein.

The terms “nucleic acid molecule” and “polynucleotide” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

As used herein, the term “a reagent that specifically detects expression levels” refers to one or more reagents used to detect the expression level of one or more biomarkers (e.g., a polypeptide selected from the marker proteins provided herein, a nucleic acid molecule comprising a polynucleotide sequence coding for a marker protein, or a polynucleotide that hybridizes with at least a portion of the nucleic acid molecule). Examples of suitable reagents include, but are not limited to, antibodies capable of specifically binding to a marker protein of interest, nucleic acid probes capable of specifically hybridizing to a polynucleotide sequence of interest, or PCR primers capable of specifically amplifying a polynucleotide sequence of interest. The term “amplify” is used herein in the broad sense to mean creating/generating an amplification product. “Amplification”, as used herein, generally refers to the process of producing multiple copies of a desired sequence, particularly those of a sample. A “copy” does not necessarily mean perfect sequence complementarity or identity to the template sequence.

The term “hybridizing” refers to the binding of two single stranded nucleic acids via complementary base pairing. The term “specific hybridization” refers to a process in which a nucleic acid molecule preferentially binds, duplexes, or hybridizes to a particular nucleic acid sequence under stringent conditions (e.g., in the presence of competitor nucleic acids with a lower degree of complementarity to the hybridizing strand). In certain embodiments of the present invention, these terms more specifically refer to a process in which a nucleic acid fragment (or segment) from a test sample preferentially binds to a particular probe and to a lesser extent or not at all, to other probes, for example, when these probes are immobilized on an array.

The terms “array”, “micro-array”, and “biochip” are used herein interchangeably. They refer to an arrangement, on a substrate surface, of hybridizable array elements, preferably, multiple nucleic acid molecules of known sequences. Each nucleic acid molecule is immobilized to a discrete spot (i.e., a defined location or assigned position) on the substrate surface. The term “micro-array” more specifically refers to an array that is miniaturized so as to require microscopic examination for visual evaluation.

The term “probe”, as used herein, refers to a nucleic acid molecule of known sequence, which can be a short DNA sequence (i.e., an oligonucleotide), a PCR product, or mRNA isolate. Probes are specific DNA sequences to which nucleic acid fragments from a test sample are hybridized. Probes specifically bind to nucleic acids of complementary or substantially complementary sequence through one or more types of chemical bonds, usually through hydrogen bond formation.

The terms “labeled”, “labeled with a detectable agent” and “labeled with a detectable moiety” are used herein interchangeably. These terms are used to specify that an entity (e.g., a probe) can be visualized, for example, following binding to another entity (e.g., a polynucleotide or polypeptide). Preferably, the detectable agent or moiety is selected such that it generates a signal which can be measured and whose intensity is related to the amount of bound entity. In array-based methods, the detectable agent or moiety is also preferably selected such that it generates a localized signal, thereby allowing spatial resolution of the signal from each spot on the array. Methods for labeling polypeptides or polynucleotides are well-known in the art. Labeled polypeptides or polynucleotides can be prepared by incorporation of or conjugation to a label, that is directly or indirectly detectable by spectroscopic, photochemical, biochemical, immunochemical, electrical, optical, or chemical means. Suitable detectable agents include, but are not limited to, various ligands, radionuclides, fluorescent dyes, chemiluminescent agents, microparticles, enzymes, calorimetric labels, magnetic labels, and haptens. Detectable moieties can also be biological molecules such as molecular beacons and aptamer beacons.

The term “OA expression profile map” refers to a presentation of expression levels of a set of biomarkers in a particular status of OA (e.g., absence of disease, OA, early OA and late OA). The map may be presented as a graphical representation (e.g., on paper or a computer screen), a physical representation (e.g., a gel or array) or a digital representation stored in a computer-readable medium. Each map corresponds to a particular status of the disease (e.g., absence of disease, OA, early OA and late OA), and thus provides a template for comparison to a patient sample. In certain preferred embodiments, maps are generated from a plurality of samples obtained from a significant number of normal individuals or individuals with the same stage/status of OA. Maps may be established for individuals with matched age, sex and body mass index.

The term “computer readable medium” refers to any device or system for storing or providing information (e.g., data and instructions) to a computer processor. Examples of computer readable media include, but are not limited to, DVDs, CDs, hard disk drives, magnetic tape and servers for streaming media over networks.

The terms “compound” and “agent” are used herein interchangeably. They refer to any naturally occurring or non-naturally occurring (i.e., synthetic or recombinant) molecule, such as a biological macromolecule (e.g., nucleic acid, polypeptide or protein), organic or inorganic molecule, or an extract made from biological materials such as bacteria, plants, fungi, or animal (particularly mammalian, including human) cells or tissues. The compound may be a single molecule or a mixture or complex of at least two molecules.

The term “candidate compound” refers to a compound or agent (as defined above) that is to be tested for an activity of interest. In the screening methods of the present invention, candidate compounds are evaluated for their ability to modulate (e.g., increase or decrease) the expression level of one or more of the biomarkers provided herein. Particularly interesting are candidate compounds that can restore the expression profile of one or more disease indicative biomarkers of a patient with OA to an expression profile more similar to that of an individual afflicted with an earlier stage of the disease or to that of a normal individual. Such compounds are potential “OA therapeutic agents”.

As used herein, the term “effective amount” refers to an amount of a compound or agent that is sufficient to fulfill its intended purpose(s). In the context of the present invention, the purpose(s) may be, for example: to modulate the expression of at least one inventive biomarker; and/or to delay or prevent the onset of OA; and/or to slow down or stop the progression, aggravation, or deterioration of the symptoms of OA; and/or to bring about amelioration of the symptoms of OA, and/or to cure OA.

The term “system” and “biological system” are used herein interchangeably. A system may be any biological entity that can express or comprise at least one inventive biomarker. In the context of the present invention, in vitro, in vivo, and ex vivo systems are considered; and the system may be a cell, a biological fluid, a biological tissue, or an animal. For example, a system may originate from a living subject (e.g., it may be obtained by drawing blood, or by performing needle biopsy), or from a deceased subject (e.g., it may be obtained at autopsy).

A “pharmaceutical composition” is defined herein as comprising at least one compound of the invention (i.e., a candidate compound identified by an inventive screening method as a modulator of the expression of at least one inventive biomarker), and at least one pharmaceutically acceptable carrier.

As used herein, the term “pharmaceutically acceptable carrier” refers to a carrier medium which does not interfere with the effectiveness of the biological activity of the active ingredients and which is not excessively toxic to the host at the concentrations at which it is administered. The term includes solvents, dispersion media, coatings, antibacterial and antifungal agents, isotonic agents, absorption delaying agents, and the like. The use of such media and agents for pharmaceutically active substances is well known in the art (see, for example, Remington's Pharmaceutical Sciences, E. W. Martin, 18th Ed., 1990, Mack Publishing Co., Easton, Pa.).

The term “treatment” is used herein to characterize a method that is aimed at (1) delaying or preventing the onset of OA; or (2) slowing down or stopping the progression, aggravation, or deterioration of the symptoms of the condition; or (3) bringing about ameliorations of the symptoms of the condition; or (4) curing the condition. A treatment may be administered prior to the onset of the disease, for a prophylactic or preventive action. It may also be administered after initiation of the disease, for a therapeutic action.

The present invention relates to improved systems and strategies for the diagnostic, characterization, and staging of OA. In particular, the present invention provides the identity of biomarkers whose expression has been found to correlate with OA and OA progression.

In one aspect, the present invention provides the identity of a set of proteins indicative of OA identified using high-throughput proteomics technology. The protein markers indicative of OA are listed in Tables 1, 2, 3, 4, 5, and 6. More specifically, by analyzing samples of serum protein depleted, age-matched synovial fluid obtained from healthy patients and from patients with early OA or late OA, it was found that the proteins listed in Tables 1, 2, 3, 4, 5 and 6 can be used to discriminate between normal/healthy and early OA and normal/healthy and late OA. It was also found that the proteins listed in Tables 1, 2, 3, 4, 5 and 6 can be used to discriminate between early OA, late OA, and healthy individuals.

The samples of synovial fluid obtained from patients with early and late OA compared to samples of synovial fluid obtained from normal individuals exhibit differing expression levels (i.e., increased expression levels and decreased levels of expression) of the proteins listed of the proteins listed in Tables 1, 2, 3, 4, 5, and 6. Therefore, the expression profiles of the proteins presented in Tables 1, 2, 3, 4, 5, and 6 can be used to diagnose OA as well as to determine the presence and/or degree of advancement of the disease (i.e., to determine the stage of the disease).

In one embodiment, the proteins can comprise at least one of alpha-2-macroglobulin precursor, fibronectin precursor, Chain B structure of complement C3b, C9 complement protein, coagulation factor II precursor, alpha-1-antichymotrypsin, complement component C3, complement factor H, inter-alpha-trypsin heavy chain H1 precursor, complement factor H isoform a precursor, gelsolin isoform a precursor, ceruloplasmin, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In other embodiments, the proteins can comprise at least one of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), 5100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In still other embodiments, the proteins can comprise at least one of fibronectin precursor, complement component C3, alpha-2-macroglobulin precursor, Chain B Structure of Complement C3b, complement factor H, fibronectin 1 isoform 3 preproprotein, alpha-2-macroglobulin, collagen type IV alpha 1, alph 2 type IV collagen preprotein, inter-alpha-trypsin inhibitor, C-terminal inter-alpha trypsin inhibitor, ceruloplasmin, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, serum albumin, COMP, ALB protein, gelsolin isoform a precursor, complement factor B, fibulin-1 isoform D precursor, inter-alpha-globulin inhibitor H4, valosin-containing protein, Vitamin D-binding protein precursor, complement component 2 precursor, annexin A2, Annexin A2 isoform 2, homerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

In further embodiments, the proteins can comprise at least one of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, homerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

In still other embodiments, the proteins can comprise at least one of alpha-2-macroglobulin precursor, fibronectin precursor, Chain B structure of complement C3b, C9 complement protein, coagulation factor II precursor, alpha-1-antichymotrypsin, complement component C3, complement factor H, inter-alpha-trypsin heavy chain H1 precursor, complement factor H isoform a precursor, gelsolin isoform a precursor, ceruloplasmin, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, fibronectin 1 isoform 3 preproprotein, alpha-2-macroglobulin, collagen type IV alpha 1, C-terminal inter-alpha trypsin inhibitor, phosphatidylinositol-glycan-specific phospholipase D1, complement component 6 isoform CRA_b, Chain A Crystal Structure of human Galectin-7, serum albumin, ALB protein, inter-alpha-globulin inhibitor H4, valosin-containing protein, Vitamin D-binding protein precursor, complement component 2 precursor, annexin A2, homerin precursor, Protein S100-A7 (psoriasin), coagulation factor XIII, B Chain Alpha-Ferrous-Carbonmonoxy, insulin-like growth factor binding protein acid labile, histidine-rich glycoprotein precursor, phospholipid transfer protein isoform a precursor, coagulation factor XIII B chain, vitronectin, biotinidase precursor, alpha-1-B-glycoprotein, Coagulation factor XIII B chain precursor, vanin 1 precursor, extracellular matrix protein 1 isoform precursor, histidine-rich glycoprotein, dopamine beta-hydroxylase precursor, Chain A Crystal Structure of Native Heparin Cofactor Ii, inter-alpha (globulin) inhibitor H1, vitronectin precursor, Vitamin K-dependent protein S precursor, GRP78, analogs and fragments thereof; and any combination thereof.

In other embodiments, the proteins can comprise at least one of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), 5100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, fibronectin 1 isoform 3 preproprotein, collagen type IV alpha 1, C-terminal inter-alpha trypsin inhibitor, phosphatidylinositol-glycan-specific phospholipase D1, complement component 6 isoform CRA_b, Chain A Crystal Structure of human Galectin-7, ALB protein, valosin-containing protein, Vitamin D-binding protein precursor, complement component 2 precursor, annexin A2, homerin precursor, Protein S100-A7 (psoriasin), coagulation factor XIII, B Chain Alpha-Ferrous-Carbonmonoxy, insulin-like growth factor binding protein acid labile, histidine-rich glycoprotein precursor, phospholipid transfer protein isoform a precursor, coagulation factor XIII B chain, vitronectin, biotinidase precursor, alpha-1-B-glycoprotein, Coagulation factor XIII B chain precursor, vanin 1 precursor, extracellular matrix protein 1 isoform precursor, histidine-rich glycoprotein, dopamine beta-hydroxylase precursor, Chain A Crystal Structure of Native Heparin Cofactor Ii, inter-alpha (globulin) inhibitor H1, vitronectin precursor, Vitamin K-dependent protein S precursor, GRP78, analogs and fragments thereof; and any combination thereof.

Other OA biomarkers provided by the present invention include nucleic acid molecules comprising polynucleotide sequences coding for the inventive protein markers described above (or analogs and fragments thereof) and polynucleotides that hybridize with portions of these nucleic acid molecules.

Information on expression levels of a given set of biomarkers obtained using biological samples from individuals afflicted with a particular stage of the disease (e.g., healthy subjects, patients with OA, with early OA, or with late OA) may be grouped to form an OA expression profile map. Preferably, an OA expression profile map results from the study of a large number of individuals with the same disease stage/status. In certain embodiments, an OA expression profile map is established using samples from individuals with matched age, sex, and body index. Each expression profile map provides a template for comparison to biomarker expression patterns generated from unknown biological samples. OA expression profile maps may be presented as a graphical representation (e.g., on paper or a computer screen), a physical representation (e.g., a gel or array) or a digital representation stored in a computer-readable medium.

As will be appreciated by those of ordinary skill in the art, sets of biomarkers whose expression profiles correlate with OA, can discriminate between different stages of the disease may be used to identify, study or characterize unknown biological samples. Accordingly, the present invention provides methods for characterizing biological samples obtained from a subject suspected of having OA, for diagnosing OA in a subject, and for assessing the advancement of OA in a subject. In such methods, the biomarkers' expression levels determined for a biological sample obtained from the subject are compared to the levels in one or more control samples. The control samples may be obtained from a healthy individual (or a group of healthy individuals), from an individual (or group of individuals) afflicted with OA, and/or from an individual (or group of individuals) afflicted with a specific stage of the disease (e.g., early OA or late OA). As mentioned above, the control expression levels of the biomarkers of interest are preferably determined from a significant number of individuals, and an average or mean is obtained. In certain preferred embodiments, the expression levels determined for the biological sample under investigation are compared to at least one expression profile map for OA, as described above.

In certain embodiments, the control protein expression profile is an early stage osteoarthritis expression profile, and the difference is indicative of late stage osteoarthritis. In such embodiments, the difference may be selected from the group consisting of an increase or decrease in the level of expression of one or more proteins selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In other embodiments, the control protein expression profile is a late stage osteoarthritis expression profile, and the difference is indicative of early stage osteoarthritis. In such embodiments, the difference may be selected from the group consisting of an increase or decrease in the level of expression of one or more proteins selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

In other embodiments, the control protein expression profile is a healthy or normal expression profile, and the difference is indicative of early or late stage osteoarthritis. In such embodiments, the difference may be selected from the group consisting of an increase or decrease in the level of expression of one or more proteins selected from the group consisting of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, hornerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

In still other embodiments, the control protein expression profile is a late or early stage osteoarthritis expression profile, and the difference is indicative of normal or healthy subject. In such embodiments, the difference may be selected from the group consisting of an increase or decrease in the level of expression of one or more proteins selected from the group consisting of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, hornerin precursor, complement component 1 subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

The methods of the invention may be applied to the study of any type of biological samples allowing one or more inventive biomarkers to be assayed. Examples of biological samples include, but are not limited to, urine, blood, blood products, joint fluid, saliva, and synovial fluid. The biological samples used in the practice of the inventive methods of diagnostic may be fresh or frozen samples collected from a subject, or archival samples with known diagnosis, treatment and/or outcome history. Biological samples may be collected by any non-invasive means, such as, for example, by drawing blood from a subject, or using fine needle aspiration or needle biopsy. Alternatively, biological samples may be collected by an invasive method, including, for example, surgical biopsy.

In certain embodiments, the inventive methods are performed on the biological sample itself without or with limited processing of the sample.

In other embodiments, the inventive methods are performed at the single cell level (e.g., isolation of cells from the biological sample). However, in such embodiments, the inventive methods are preferably performed using a sample comprising many cells, where the assay is “averaging” expression over the entire collection of cells present in the sample. Preferably, there is enough of the biological sample to accurately and reliably determine the expression of the set of biomarkers of interest. Multiple biological samples may be taken from the same tissue/body part in order to obtain a representative sampling of the tissue.

In still other embodiments, the inventive methods are performed on a protein extract prepared from the biological sample. Preferably, the protein extract contains the total protein content. However, the methods may also be performed on extracts containing one or more of: membrane proteins, nuclear proteins, and cytosolic proteins. Methods of protein extraction are well known in the art (see, for example “Protein Methods”, D. M. Bollag et al., 2nd Ed., 1996, Wiley-Liss; “Protein Purification Methods: A Practical Approach”, E. L. Harris and S. Angal (Eds.), 1989; “Protein Purification Techniques: A Practical Approach”, S. Roe, 2nd Ed., 2001, Oxford University Press; “Principles and Reactions o/Protein Extraction, Purification, and Characterization”, H. Ahmed, 2005, CRC Press: Boca Raton, Fla.). Numerous different and versatile kits can be used to extract proteins from bodily fluids and tissues, and are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kites) most appropriate for a particular situation. After the protein extract has been obtained, the protein concentration of the extract is preferably standardized to a value being the same as that of the control sample in order to allow signals of the protein markers to be quantitated. Such standardization can be made using photometric or spectrometric methods or gel electrophoresis.

In yet other embodiments, the inventive methods are performed on nucleic acid molecules extracted from the biological sample. For example, RNA may be extracted from the sample before analysis. Methods of RNA extraction are well known in the art (see, for example, J. Sambrook et al., “Molecular Cloning: A Laboratory Manual”, 1989, 2nd Ed., Cold Spring Harbor Laboratory Press: Cold Spring Harbor, N.Y.). Most methods of RNA isolation from bodily fluids or tissues are based on the disruption of the tissue in the presence of protein denaturants to quickly and effectively inactivate RNAses. Isolated total RNA may then be further purified from the protein contaminants and concentrated by selective ethanol precipitations, phenol/chloroform extractions followed by isopropanol precipitation or cesium chloride, lithium chloride or cesium trifluoroacetate gradient centrifugations. Kits are also available to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Qiagen, Inc. (Valencia, Calif.).

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each probe being monitored, or using thermostable DNApolymerases (such as avian myeloblastosis virus reverse transcriptase or Moloney murine leukemia virus reverse transcriptase).

The diagnostic methods of the present invention generally involve the determination of expression levels of a plurality (i.e., one or more, e.g., at least 2, at least 3, at least 4, at least 5, at least 6, at least 7, at least 8, at least 9, at least 10 or more) of polypeptides in a biological sample obtained from a subject. Determination of protein expression levels in the practice of the inventive methods may be performed by any suitable method (see, for example, E. Harlow and A. Lane, “Antibodies: A Laboratories Manual”, 1988, Cold Spring Harbor Laboratory Cold Spring Harbor, N.Y.).

In general, protein expression levels are determined by contacting a biological sample isolated from a subject with binding agents for one or more of the protein markers; detecting, in the sample, the levels of polypeptides that bind to the binding agents; and comparing the levels of polypeptides in the sample with the levels of polypeptides in a control sample. As used herein, the term “binding agent” refers to an entity such as a polypeptide or antibody that specifically binds to an inventive protein marker. An entity “specifically binds” to a polypeptide if it reacts/interacts at a detectable level with the polypeptide but does not react/interact detectably with peptides containing unrelated sequences or sequences of different polypeptides.

In certain embodiments, the binding agent is a ribosome, with or without a peptide component, an RNA molecule, or a polypeptide (e.g., a polypeptide that comprises a polypeptide sequence of a protein marker, a peptide variant thereof, or a non-peptide mimetic of such a sequence).

In other embodiments, the binding agent is an antibody specific for a protein marker of the invention. Suitable antibodies for use in the methods of the present invention include monoclonal and polyclonal antibodies, immunologically active fragments (e.g., Fab or (Fab)₂ fragments), antibody heavy chains, humanized antibodies, antibody light chains, and chimeric antibodies. Antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czemik et al., NeuroNeuroprotocols, 1995, 6: 56-61; H. Zhang et al., J. Biol. Chem. 2002, 277: 39379-39387; S. L. Morrison et al., Proc. Natl. Acad. Sci., 1984, 81: 6851-6855; M. S. Neuberger et al., Nature, 1984, 312: 604-608; S. Takeda et al., Nature, 1985, 314: 452-454). Antibodies to be used in the methods of the invention can be purified by methods well known in the art (see, for example, S. A. Minden, “Monoclonal Antibody Purification”, 1996, IBC Biomedical Library Series: Southbridge, Mass.). For example, antibodies can be affinity purified by passage over a column to which a protein marker or fragment thereof is bound. The bound antibodies can then be eluted from the column using a buffer with a high salt concentration.

Instead of being prepared, antibodies to be used in the methods of the present invention may be obtained from scientific or commercial sources.

In certain embodiments, the binding agent is directly or indirectly labeled with a detectable moiety. The role of a detectable agent is to facilitate the detection step of the diagnostic method by allowing visualization of the complex formed by binding of the binding agent to the protein marker (or analog or fragment thereof). Preferably, the detectable agent is selected such that it generates a signal which can be measured and whose intensity is related (preferably proportional) to the amount of protein marker present in the sample being analyzed. Methods for labeling biological molecules such as polypeptides and antibodies are well-known in the art (see, for example, “Affinity Techniques. Enzyme Purification. Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

In certain embodiments, the binding agents (e.g., antibodies) may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinylether-maleic acid copolymer, amino acid copolymer, ethylene-maleic acid copolymer, nylon, silk, and the like. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

Protein expression levels in the diagnostic methods of the present invention may be determined using immunoassays. Examples of such assays are radioimmunoassays, enzyme immunoassays (e.g., ELISA), immunofluorescence immunoprecipitation, latex agglutination, hemagglutination, and histochemical tests, which are conventional methods well-known in the art. As will be appreciated by one skilled in the art, the immunoassay may be competitive or noncompetitive. Methods of detection and quantification of the signal generated by the complex formed by binding of the binding agent with the protein marker will depend on the nature of the assay and of the detectable moiety (e.g., fluorescent moiety).

Alternatively, the protein expression levels may be determined using mass spectrometry based methods or image (including use of labeled ligand) based methods known in the art for the detection of proteins. Other suitable methods include proteomics-based methods. Proteomics, which studies the global changes of protein expression in a sample, typically includes the following steps: (I) separation of individual proteins in a sample by electrophoresis (2-D PAGE), (2) identification of individual proteins recovered from the gel (e.g., by mass spectrometry or N-terminal sequencing), and (3) analysis of the data using bioinformatics.

As already mentioned above, the diagnostic methods of the present invention may involve determination of the expression levels of a set of nucleic acid molecules comprising polynucleotide sequences coding for an inventive protein marker. Determination of expression levels of nucleic acid molecules in the practice of the inventive methods may be performed by any suitable method, including, but not limited to, Southern analysis, Northern analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds.), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320308), one-sided PCR (Ohara et al., Proc. Natl. Acad. Sci., 1989, 86: 5673-5677), in situ hybridization, Taqman based assays (Holland et al., Proc. Natl. Acad. Sci., 1991, 88:7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan™, and the like.

Nucleic acid probes for use in the detection of polynucleotide sequences in biological samples may be constructed using conventional methods known in the art. Suitable probes may be based on nucleic acid sequences encoding at least 5 sequential amino acids from regions of nucleic acids encoding a protein marker, and preferably comprise about 15 to about 50 nucleotides. A nucleic acid probe may be labeled with a detectable moiety, as mentioned above in the case of binding agents. The association between the nucleic acid probe and detectable moiety can be covalent or non-covalent. Detectable moieties can be attached directly to nucleic acid probes or indirectly through a linker (E. S. Mansfield et al., Mol. Cell. Probes, 1995, 9: 145-156). Methods for labeling nucleic acid molecules are well-known in the art (for a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35:135-153).

Nucleic acid probes may be used in hybridization techniques to detect polynucleotides encoding the protein markers. The technique generally involves contacting an incubating nucleic acid molecules in a biological sample obtained from a subject with the nucleic acid probes under conditions such that specific hybridization takes place between the nucleic acid probes and the complementary sequences in the nucleic acid molecules. After incubation, the non-hybridized nucleic acids are removed, and the presence and amount of nucleic acids that have hybridized to the probes are detected and quantified.

Detection of nucleic acid molecules comprising polynucleotide sequences coding for a protein marker may involve amplification of specific polynucleotide sequences using an amplification method such as PCR, followed by analysis of the amplified molecules using techniques known in the art. Suitable primers can be routinely designed by one skilled in the art. In order to maximize hybridization under assay conditions, primers and probes employed in the methods of the invention generally have at least 60%, preferably at least 75% and more preferably at least 90% identity to a portion of nucleic acids encoding a protein marker.

Hybridization and amplification techniques described herein may be used to assay qualitative and quantitative aspects of expression of nucleic acid molecules comprising polynucleotide sequences coding for the inventive protein markers.

Alternatively, oligonucleotides or longer fragments derived from nucleic acids encoding each protein marker may be used as targets in a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; 1. 1. Chen et al., Genomics, 1998, 51: 313324; U.S. Pat. Nos. 5,143,854; 5,445,934; 5,807,522; 5,837,832; 6,040,138; 6,045,996; 6,284,460; and 6,607,885).

Once the expression levels of the biomarkers of interest have been determined (as described above) for the biological sample being analyzed, they are compared to the expression levels in one or more control samples or to at least one expression profile map for OA. Comparison of expression levels according to methods of the present invention is preferably performed after the expression levels obtained have been corrected for both differences in the amount of sample assayed and variability in the quality of the sample used (e.g., amount of protein extracted, or amount and quality of mRNA tested). Correction may be carried out using different methods well-known in the art. For example, the protein concentration of a sample may be standardized using photometric or spectrometric methods or gel electrophoresis (as already mentioned above) before the sample is analyzed. In case of samples containing nucleic acid molecules, correction may be carried out by normalizing the levels against reference genes (e.g., housekeeping genes) in the same sample. Alternatively or additionally, normalization can be based on the mean or median signal (e.g., Ct in the case of RT-PCR) of all assayed genes or a large subset thereof (global normalization approach).

For a given set of biomarkers, comparison of an expression pattern obtained for a biological sample against an expression profile map established for a particular stage of OA may comprise comparison of the normalized expression levels on a biomarker-by-biomarker basis and/or comparison of ratios of expression levels within the set of biomarkers. In addition, the expression pattern obtained for the biological sample being analyzed, may be compared against each of the expression profile maps (e.g., expression profile map for non-OA, expression profile map for OA, expression profile map for early OA, and expression profile map for late OA) or against an expression profile that defines delineations made based upon all the OA expression profile maps.

Using methods described herein, skilled physicians may select and prescribe treatments adapted to each individual patient based on the diagnosis and disease staging provided to the patient through determination of the expression levels of the inventive biomarkers. In particular, the present invention provides physicians with a non-subjective means to diagnose early OA, which will allow for early treatment, when intervention is likely to have its greatest effect, potentially preventing pain and long-term disability and improving patient's quality of life. Selection of an appropriate therapeutic regimen for a given patient may be made based solely on the diagnosis/staging provided by the inventive methods. Alternatively, the physician may also consider other clinical or pathological parameters used in existing methods to diagnose OA and assess its advancement.

Furthermore, the methods of OA diagnosis and OA staging provided by the present invention allow the disease to be monitored even when signs of cartilage destruction would not be visible or when changes in joint spaces would not be detectable on X-ray images.

In another aspect, the present invention provides kits comprising materials useful for carrying out diagnostic methods according to the present invention. The diagnosis and staging procedures described herein may be performed by diagnostic laboratories, experimental laboratories, or practitioners. The invention provides kits, which can be used in these different settings.

Materials and reagents for characterizing biological samples, diagnosing OA in a subject, and/or staging OA in a subject according to the inventive methods may be assembled together in a kit. In certain embodiments, an inventive kit comprises at least one reagent that specifically detects expression levels of one or more inventive biomarkers, and instructions for using the kit according to a method of the invention. Each kit may preferably include the reagent, which renders the procedure specific. Thus, for detecting/quantifying a protein marker (or an analog or fragment thereof), the reagent that specifically detects expression levels of the protein may be an antibody that specifically binds to the protein marker (or analog or fragment thereof). For detecting/quantifying a nucleic acid molecule comprising a polynucleotide sequence coding a protein marker, the reagent that specifically detects expression levels may be a nucleic acid probe complementary to the polynucleotide sequence (e.g., cDNA or an oligonucleotide). The nucleic acid probe may or may not be immobilized on a substrate surface (e.g., beads, a microarray, and the like).

Depending on the procedure, the kit may further comprise one or more of, extraction buffer and/or reagents, amplification buffer and/or reagents, hybridization buffer and/or reagents, immunodetection buffer and/or reagents, labeling buffer and/or reagents, and detection means. Protocols for using these buffers and reagents for performing different steps of the procedure may be included in the kit.

The reagents may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, the kits of the present invention further comprise control samples. In other embodiments, the inventive kits comprise at least one expression profile map for OA and/or OA progression as described herein for use as comparison template. Preferably, the expression profile map is digital information stored in a computer-readable medium.

Instructions for using the kit, according to one or more methods of the invention, may comprise instructions for processing the biological sample obtained from the subject, and/or for performing the test, instructions for interpreting the results. As well as a notice in the form prescribed by a governmental agency (e.g., FDA) regulating the manufacture, use or sale of pharmaceuticals or biological products.

As noted above, the inventive biomarkers whose expression profiles correlate with osteoarthritis and/or osteoarthritis progression are attractive targets for the identification of new therapeutic agents (e.g., using screens to detect compounds or substances that inhibit or enhance the expression of these biomarkers). Accordingly, the present invention provides methods for the identification of compounds potentially useful for treating osteoarthritis or modulating osteoarthritis progression.

The inventive methods comprise incubating a biological system, which expresses at least one inventive biomarker, with a candidate compound under conditions and for a time sufficient for the candidate compound to modulate the expression of the biomarker, thereby obtaining a test system; incubating the biological system under the same conditions and for the same time absent the candidate compound, thereby obtaining a control system; measuring the expression level of the biomarker in the test system; measuring the expression level of the biomarker in the control system; and determining that the candidate compound modulates the expression of the biomarker if the expression level measured in the test system is less than or greater than the expression level measured in the control system.

The assay and screening methods of the present invention may be carried out using any type of biological systems, e.g., a cell or cells, a biological fluid, a biological tissue, or an animal. In certain embodiments, the methods are carried out using a system that can exhibit cartilage degeneration due to OA (e.g., an animal model, or whole or portion of a body part, e.g., the knee). In other embodiments, the methods are carried out using a biological entity that expresses or comprises at least one inventive biomarker (e.g., a cell or a sample of blood, urine, saliva, or synovial fluid).

In certain preferred embodiments, the assay and screening methods of the present invention are carried out using cells that can be grown in standard tissue culture plastic ware. Such cells include all appropriate normal and transformed cells derived from any recognized sources. Preferably, cells are of mammalian (human or animal, such as rodent or simian) origin. More preferably, cells are of human origin. Mammalian cells may be of any organ or tissue origin (e.g., bone, cartilage, or synovial fluid) and of any cell types as long as the cells express at least one inventive biomarker.

Cells to be used in the practice of the methods of the present invention may be primary cells, secondary cells, or immortalized cells (e.g., established cell lines). They may be prepared by techniques well known in the art (for example, cells may be isolated from bone, cartilage or synovial fluid) or purchased from immunological and microbiological commercial resources (for example, from the American Type Culture Collection, Manassas, Va.). Alternatively or additionally, cells may be genetically engineered to contain, for example, a gene of interest.

Selection of a particular cell type and/or cell line to perform an assay according to the present invention will be governed by several factors such as the nature of the biomarker whose expression is to be modulated and the intended purpose of the assay. For example, an assay developed for primary drug screening (i.e., first round(s) of screening) is preferably performed using established cell lines, which are commercially available and usually relatively easy to grow, while an assay to be used later in the drug development process is preferably performed using primary and secondary cells, which are generally more difficult to obtain, maintain and/or grow than immortalized cells but which represent better experimental models for in vivo situation. Examples of established cell lines that can be used in the practice of the assay and screening methods of the present invention include fibroblastic and/or osseously derived cell lines. Primary and secondary cells that can be used in the inventive screening methods include, but are not limited to, chondrocytes and osteocytes.

Cells to be used in the inventive assays may be cultured according to standard cell culture techniques. For example, cells are often grown in a suitable vessel in a sterile environment at 37° C. in an incubator containing a humidified 95% air-5% CO₂ atmosphere. Vessels may contain stirred or stationary cultures. Various cell culture media may be used including media containing undefined biological fluids such as fetal calf serum. Cell culture techniques are well known in the art and established protocols are available for the culture of diverse cell types (see, for example, R. I. Freshney, “Culture of Animal Cells: A Manual of Basic Technique”, 2nd Edition, 1987, Alan R. Liss, Inc.).

In certain embodiments, the screening methods are performed using cells contained in a plurality of wells of a multi-well assay plate. Such assay plates are commercially available, for example, from Stratagene Corp. (La Jolla, Calif.) and Coming Inc. (Acton, Mass.) and include, for example, 48-well, 96-well, 384-well and 1536-well plates.

As will be appreciated by those of ordinary skill in the art, any kind of compounds or agents can be tested using the inventive methods. A candidate compound may be a synthetic or natural compound; it may be a single molecule or a mixture or complex of different molecules. In certain embodiments, the inventive methods are used for testing one or more compounds. In other embodiments, the inventive methods are used for screening collections or libraries of compounds. As used herein, the term “collection” refers to any set of compounds, molecules or agents, while the term “library” refers to any set of compounds, molecules or agents that are structural analogs.

Collections of natural compounds in the form of bacterial, fungal, plant and animal extracts are available from, for example, Pan Laboratories (Bothell, Wash.) or MycoSearch (Durham, N.C.). Libraries of candidate compounds that can be screened using the methods of the present invention may be either prepared or purchased from a number of companies. Synthetic compound libraries are commercially available from, for example, Comgenex (Princeton, N.J.), Brandon Associates (Merrimack, N.H.), Microsource (New Milford, Conn.), and Aldrich (Milwaukee, Wis.). Libraries of candidate compounds have also been developed by and are commercially available from large chemical companies, including, for example, Merck, Glaxo Welcome, Bristol-Meyers-Squibb, Novartis, Monsanto/Searle, and Pharmacia UpJohn. Additionally, natural collections, synthetically produced libraries and compounds are readily modified through conventional chemical, physical, and biochemical means. Chemical libraries are relatively easy to prepare by traditional automated synthesis, PCR, cloning or proprietary synthetic methods (see, for example, S. H. DeWitt et al., Proc. Natl. Acad. Sci. U.S.A. 1993, 90:6909-6913; R. N. Zuckermann et al., J. Med. Chem. 1994, 37: 2678-2685; Carell et al., Angew. Chem. Int. Ed. Engl. 1994, 33: 2059-2060; P. L. Myers, Curro Opin. Biotechnol. 1997, 8: 701-707).

Useful agents for the treatment of osteoarthritis may be found within a large variety of classes of chemicals, including heterocycles, peptides, saccharides, steroids, and the like. In certain embodiments, the screening methods of the invention are used for identifying compounds or agents that are small molecules (i.e., compounds or agents with a molecular weight <600-700 Da).

The screening of libraries according to the inventive methods will provide “hits” or “leads”, i.e., compounds that possess a desired but not-optimized biological activity. The next step in the development of useful drug candidates is usually the analysis of the relationship between the chemical structure of a hit compound and its biological or pharmacological activity. Molecular structure and biological activity are correlated by observing the results of systemic structural modification on defined biological end-points. Structure-activity relationship information available from the first round of screening can then be used to generate small secondary libraries, which are subsequently screened for compounds with higher affinity. The process of performing synthetic modifications of a biologically active compound to fulfill all stereoelectronic, physicochemical, pharmacokinetic, and toxicologic factors required for clinical usefulness is called lead optimization. Candidate compounds identified as potential OA therapeutic agents by screening methods of the present invention can similarly be subjected to a structure-activity relationship analysis, and chemically modified to provide improved drug candidates. The present invention also encompasses these improved drug candidates.

In the screening methods of the present invention, a candidate compound is identified as a modulator of the expression of at least one inventive biomarker if the expression level of the biomarker in the test sample is lower or greater than the expression level of the same biomarker in the control sample. Reproducibility of the results obtained using methods of the present invention may be tested by performing the analysis more than once with the same concentration of the same candidate compound (for example, by incubating cells in more than one well of an assay plate). Additionally, since candidate compounds may be effective at varying concentrations depending on the nature of the compound and the nature of its mechanism(s) of action, varying concentrations of the candidate compound may be tested (for example, by addition of different concentrations of the candidate compound in different wells containing cells in an assay plate). Generally, candidate compound concentrations from about 1 μM to about 10 mM are used for screening. Preferred screening concentrations are between about 10 μM and about 100 μM.

In certain embodiments, the methods of the invention further involve the use of one or more negative or positive control compounds. A positive control compound may be any molecule or agent that is known to modulate the expression of at least one biomarker studied in the screening assay. A negative control compound may be any molecule or agent that is known to have no detectable effects on the expression of at least one biomarker studied in the screening assay. In these embodiments, the inventive methods further comprise comparing the modulating effects of the candidate compound to the modulating effects (or absence thereof) of the positive (or negative) control compound.

As will be appreciated by those skilled in the art, it is generally desirable to further characterize the compounds identified by the inventive screening methods. For example, if a candidate compound has been identified as a modulator of the expression of a specific biomarker in a given cell culture system (e.g., an established cell line), it may be desirable to test this ability in a different cell culture system (e.g., primary or secondary cells). Alternatively or additionally, it may be desirable to evaluate the effects of the candidate compound on the expression of one or more other inventive biomarkers. It may also be desirable to perform pharmacokinetics and toxicology studies.

A candidate compound identified by the screening methods of the invention may also be further tested in assays that allow for the determination of the compound's properties in vivo. Suitable animal models of osteoarthritis are known in the art. In general, these models fall into two categories, spontaneous and induced (surgical instability or genetic manipulation). Animal models of naturally occurring OA occur in knee joints of guinea pigs, mice, and Syrian hamsters. Commonly used surgical instability models include medial meniscal tear in guinea pigs and rats, medial or lateral partial meniscectomy in rabbits, medial partial or total meniscectomy or anterior cruciate transection in dogs. Transgenic models have been developed in mice. Examples of animal models of osteoarthritis suitable for testing the candidate compounds identified as potential OA therapeutic agents include, but are not limited to, those described in M. J. Pond and G. Nuki, Ann. Rheum. Dis., 1973, 32: 387-388; T. Videman, Acta Orthop. Scand., 1982, 53: 339-347; S. B. Christensen, Scand. J. Rheumatol., 1983, 12: 343-349; A. M. Bendele et al., Vet. Pathol., 1987, 24: 436-443; K. D. Brandt et al., J. Rheumatol., 1991, 18: 436-446; K. D. Brandt, Ann. NY Acad. Sci., 1994, 732: 199-205; C. S. Carlson et al., J. Orthop. Res., 1994, 12: 331-339; A. G. Fam et al., Arthritis Rheum., 1995, 38: 201-210; K. W. Marshall and A. D. Chan, J. Rheumatol., 1996, 23: 344-350; H. J. Helminen et al., Rheumatol., 2002, 41: 848-856 and references cited therein; and J. L. Henry, Novartis Found Symp., 2004, 260: 139-145.

The present invention also provides pharmaceutical compositions, which comprise, as active ingredient, an effective amount of at least one compound identified by an inventive screening assay as a modulator of the expression of at least one biomarker or one set of biomarkers disclosed herein. The pharmaceutical composition may be formulated using conventional methods well known in the art. Such compositions include, in addition to the active ingredient(s), at least one pharmaceutically acceptable liquid, semi-liquid, or solid diluent acting as pharmaceutical vehicle, excipient or medium, and termed here “pharmaceutically acceptable carrier”.

According to the present invention, an inventive pharmaceutical composition may include one or more OA therapeutic agents of the invention as active ingredients. Alternatively, a pharmaceutical composition containing an effective amount of one OA therapeutic agent may be administered to a patient simultaneously with or sequentially with a pharmaceutical composition containing a different inventive OA therapeutic agent.

In another embodiment of this invention, an inventive OA therapeutic agent, or a pharmaceutical composition thereof, may be administered serially or in combination with conventional therapeutics used in the treatment of OA. Such therapeutics include pain relievers such as acetaminophen; Non-steroidal Anti-inflammatory Drugs (NSAIDs), such as aspirin, ibuprofen, naproxen, and ketoprofen; COX-2 inhibitors; corticosteroids; combination of supplement glucosamine and chondroitin sulfates; and over the counter topical formulations containing capsaicin.

Alternatively or additionally, an inventive OA therapeutic agent, or a pharmaceutical composition thereof, may be administered serially or in combination with conventional therapeutic regimens for the treatment of osteoarthritis including viscosupplementation, surgery, arthroplasty (or joint replacement surgery), arthrodesis (or joint fusion), osteotomy, arthroscopy and cartilage transplantation.

In another aspect, the present invention provides methods for the treatment and/or prevention of osteoarthritis. These methods comprise administering to a subject afflicted with OA, an effective amount of a compound that modulates the expression of at least one inventive biomarker. The compound may be known in the art to act as a modulator of the expression of the at least one biomarker. Alternatively, the compound may have been identified as an OA therapeutic agent by a screening method provided by the present invention.

Subjects suitable to receive a treatment according to the present invention include individuals that have been diagnosed with OA using conventional methods (e.g., radiological examination, clinical observations) as well as individuals that have been diagnosed with OA using diagnostic methods provided herein. Suitable subjects may or may not have previously received traditional treatment for the condition.

A treatment according to the methods of the present invention may consist of a single dose or a plurality of doses over a period of time. An inventive OA therapeutic agent, or pharmaceutical composition thereof, may also be released from a depot form per treatment. The administration may be carried out in any convenient manner such as by injection (subcutaneous, intravenous, intramuscular, intraperitoneal, or the like), oral administration, topical administration, rectal administration, or sublingual administration.

Effective dosages and administration regimens can be readily determined by good medical practice and the clinical condition of the individual patient. The frequency of administration will depend on the pharmacokinetic parameters of the active ingredient(s) and the route of administration. The optimal pharmaceutical formulation can be determined depending upon the route of administration and desired dosage. Such formulations may influence the physical state, stability, rate of in vivo release, and rate of in vivo clearance of the administered compounds.

Depending on the route of administration, a suitable dose may be calculated according to body weight, body surface area, or organ size. Optimization of the appropriate dosage can readily be made by those skilled in the art in light of pharmacokinetic data observed in human clinical trials. The final dosage regimen will be determined by the attending physician, considering various factors which modify the action of drugs, e.g., the drug's specific activity, the severity of the damage and the responsiveness of the patient, the age, condition, body weight, sex and diet of the patient, the severity of any present infection, time of administration and other clinical factors. As studies are conducted, further information will emerge regarding the appropriate dosage levels and duration of treatment for various stages of advancement of OA.

### Example

A previous study, using mass spectroscopy-based proteomic techniques in synovial fluid (SF), had identified candidate biomarkers for OA with potential to be developed as highly sensitive and specific tests for disease diagnosis. In this study, patients with OA or healthy controls had their SF fractionated using 1 dimensional SDS gels, gel bands were excised and proteins identified and quantified using mass spectrometry for OA versus control. Due to the complex nature of the SF, this technology has limited ability to identify a larger number of potential biomarkers, and in fact can be expected to be able to sift through only the top 100-200 proteins in the sample. In particular, SF contains many serum proteins that mask information relevant to the disease that could be derived from the diseased synovial tissue. Also, the analytical methods used that compared LC-MS intensities of peptides derived from gel bands in the 1-D gel analysis were not optimal for discerning significant differences, specifically they lacked accurate mass tag information.

In this study, we added a number of novel features to provide more accurate quantification and advanced statistical techniques for determining significance. We increased separation of the protein components in the sample, included a third patient group, that of early OA, and age-matched the samples. We first subjected the SF to immuno-affinity depletion to remove abundant components that are derived from admixture of synovial fluid with serum. This was intended to emphasize the contribution of proteins in the SF preparation that are differentially regulated in diseased versus normal synovial tissue. Second, instead of fractionating the proteins by a 1-D gel method, we used a 2-dimensional method that fractionates the protein by both size and charge. Coupled to the large format gels available, typically 2000-3000 protein forms can be examined for abundance variation. Third, the SF proteins were labeled with fluorescent dyes and samples from multiple groups were run on the same gel to enhance the use of modem statistical methods to examine group based differences with high confidence.

In short, we identified biomarkers in the SF of early OA and late OA patients (versus healthy controls) using Two Dimensional Fluorescence Difference Gel Electrophoresis (2D-DIGE) coupled with mass spectroscopy. Using this method, a variety of alterations that describe the progressive nature of the disease were discovered and potential candidate biomarkers for OA had been found suitable for diagnostic purposes or for evaluating therapeutic response.

Patients with Early Osteoarthritis, Late Osteoarthritis and Controls

Two separate 2D DIGE experiments with different numbers of subjects were performed in this study. In the first experiment, 4 healthy control individuals with same number of patients diagnosed with early and late OA were identified and provided SF samples (12 patients). As stated in a previous study, all samples were collected within our tertiary care referral center and approved by our hospital's institutional review board. All SF samples were snap-frozen in liquid nitrogen immediately after acquisition from the knee joint. In the second experiment, we increased the sample size to 6 controls and same number of patients diagnosed with early and late OA (18 total).

### Depletion of SF Samples and 2D DIGE Method

### Sample Preparation and Fluorescence Dye Labeling

Immuno-affinity depletion was performed for all human synovial fluid samples to remove high. abundant proteins using a commercial column from Agilent. The proteins samples were then further cleaned (GE Healthcare Clean-Up kit), and protein concentration was determined using the 2D-Quant kit as described by the manufacturer (GE Healthcare). Twelve aliquots from each sample with 25 micrograms of proteins were pooled together to prepare an internal standard.

### Gel Electrophoresis and Image Acquisition

Precast immobilized pH gradient strips (pH 3-10 NL, 24 cm) were used for isoelectric focusing and IEF was carried out on an IPGphor2 system (GE Healthcare). The proteins were separated by their pI and the strips were transferred for 2nd dimension SDS-PAGE, which separated the proteins by their molecular weight. After electrophoresis the spots labeled by Cydye in the gel were visualized using the Typhoon 9400 imager (GE Healthcare).

### Image Analysis for Differential Protein Expression

### Image Analysis by DeCyder Software

To compare protein spots across all gels, image analyses were conducted using DeCyder v6.5 2D Differential Analysis Software (GE Healthcare). Protein spot detection and quantification on a set of images was performed using Differential In•gel Analysis (DIA) module in DeCyder software. Because the internal standard was the same pooled sample within each gel, this effectively normalized all the data. Then images were loaded into the biological variation analysis (BVA) module, which matched multiple images from different gels to perform statistical analysis on differential protein expression levels between multiple groups.

### Statistical Analysis of Protein Expression

Differences in protein abundance among the three groups (Healthy, EOA and LOA) were evaluated by a one-way analysis of variance (ANOVA) considered significant at p value <0.05. Gel spots were digested by an automatic in-gel digestion system in 96-well plate and mass spectrometry analysis of the peptide for protein identification was performed on a Finnigan LTQ FT hybrid mass spectrometer (Thermo Electron Corp.).

### Database Searching and Criteria for Protein Identification

All MSIMS data derived from the IT instrument above were analyzed using Mascot Daemon (Matrix Science; version 2.2.1) using an indexed Homo sapiens (human) subset database (191437 sequences) created from the National Center for Biotechnology Information (NCBInr) non-redundant databases containing 4626804 sequences assuming the digestion enzyme as trypsin. Search parameters used in this study were: 1) fragment ion mass tolerance of 0.80 Da and peptide mass tolerance limits of 15 ppm, 2) Iodoacetamide derivative of cysteine was specified as a fixed modification (57 Da) and oxidation of methionine was specified as a variable modification (16 Da), 3) One missed cleavage site was allowed, 4) Peptide identifications were accepted at a cut off of p value as 0.05. A positive identification was accepted when a minimum of two peptide monoisotopic masses matched a particular protein with low expectation value (p<0.001).

### Results

Decyder software identified the following protein spot numbers (in bold type) as differentially expressed between Early Osteoarthritic (EOA) and Late Osteoarthritic (LOA). Listed below each protein spot number are the potential proteins that each protein spot number represents identified by Mascot search software. The criteria used to determine the results listed below include, Mr, PI, and sequence coverage. These criteria were compared between Mascot search results and Decyder image analysis. These results are listed in Appendix A which includes Tables 1-6.

Other embodiments of the invention will be apparent to those skilled in the art from a consideration of the specification or practice of the invention disclosed herein. It is intended that the specification and examples be considered as exemplary only, with the true scope of the invention being indicated by the following claims. All patents, publication, and referenced cited are incorporated by reference in their entirety.

TABLE 1

| | |
|---|---|
| 27 | alpha-2-macroglobulin precursor |
| 76 | fibronectin precursor, Chain B, Structure of complement C3b, C9 complement |
| | protein |
| 93 | no proteins meet the criteria |
| 124 | unnamed protein (coagulation factor II precursor) |
| 206 | alpha-1-antichymotrypsin |
| 210 | alpha-2-macroglobulin precursor, complement component C3, complement |
| | factor H |
| 246 | alpha-2-macroglobulin precursor, complement factor H |
| 255 | macroglobulin alpha2, complement factor H |
| 265 | No proteins meet criteria |
| 292 | alpha-2-macroglobulin precursor, complement factor H, trypsin inhibitor, inter- |
| | alpha-trypsin heavy chain H1 precursor |
| 372 | alpha-2-macroglobulin precursor, complement factor H isoforma precursor, |
| | gelsolin isoform a precursor |
| 384 | Ceruloplasmin, inter-alpha-trypsin inhibitor heavy chain H4 precursor, |
| | phosphatidylinositol-glycan-specific phospholipase D1 precursor |
| 385 | phosphatidylinositol-glycan-specific phospholipase D1 precursor, |
| | ceruloplasmin, inter-alpha-trypsin inhibitor family heavy chain-related protein |
| 393 | Ceruloplasmin, inter-alpha-trypsin inhibitor heavy chain H4 precursor, |
| | phosphatidylinositol-glycan-specific phospholipase D1 precursor |
| 394 | inter-alpha-trypsin inhibitor heavy chain H4 precursor, ceruloplasmin |
| 408 | glycosylphosphatidylinositol specific phosphatase D1, ceruloplasmin |
| 477 | alpha-2-macroglobulin precursor, complement component C6 precursor peptide, |
| | complement factor B |
| 481 | pre-pro-alpha(I) collagen |
| 495 | Ceruloplasmin, complement component C3, factor H, SERPIN2 protein, gp- |
| | 180-carboxypeptidase D-like enzyme |
| 496 | inter-alpha-trypsin inhibitor family heavy chain-related protein, Chain B, |
| | Human Complement Component C3, alpha-2-macroglobulin precursor, |
| | ceruloplasmin |
| 644 | Fibulin-1 isoform D precursor, inter-alpha-trypsin inhibitor family heavy chain- |
| | related protein, plasminogen, complement factor B, HGF activator, |
| | preproprotein |
| 746 | unnamed protein (coagulation factor II precursor), afamin precursor, Vitamin K- |
| | dependent protein, complement component 1, s subcomponent, iinter-alpha- |
| | trypsin inhibitor, ASPIC |
| 805 | complement component 3 precursor, plasma kallikrein precursor, annexin A2 |
| | isoform 2 |
| 840 | glucosamine (N-acetyl)-6-sulfatase precursor, unnamed protein (cystic fibrosis |
| | antigen), ezrin (p81) (cytovillin) (villin-2), S100 Calcium binding protein A9 |
| 849 | glucosamine (N-acetyl)-6-sulfatase precursor, coagulation factor XIII A chain |
| | precursor, peptidoglycan recognition protein L precursor, complement |
| | component 4 binding protein |
| 856 | inter-alpha-trypsin inhibitor heavy chain H1 precursor, glucosamine (N-acetyl)- |
| | 6-sulfatase precursor, fibrinogen gamma chain, coagulation factor XIII A chain |
| | precursor |
| 864 | glucosamine (N-acetyl)-6-sulfatase precursor, Ig mu chain precursor, |
| | phospholipase D3 isoform, moesin |
| 962 | complement 9, Chain B, Structure of Complement C3b, alpha-2-antiplasmin |
| | precursor, kininogen, thrombin inhibitor |
| 973 | complement 9, Chain A, antithrombin Iii, alpha-2-antiplasmin precursor, |
| | kinninogen, thrombin inhibitor, L-plastin, complement component 1, alpha-1-B- |
| | glycoprotein, hemopexin precursor |
| 1472 | complement component C4A, complement component C3, apolipoprotein A-IV |
| | precursor, serum paraoxonase/arylesterase, preprohaptoglobulin, COMP, serpin |
| | peptidase inhibitor, clade I, follistatin-like 1 precursor |
| 1498 | complement factor H-related protein 1 precursor, Chain A, Crystal Structure of |
| | Lipid-Free human Apolipoprotein A-1, annexin A2 isoform 2, phospholipase |
| | D3 isoform 2, Chain E, Structure of human transferring receptor-transferrin |
| | complex |

TABLE 2
Proteins Differentially Expressed Between EOA vs LOA (12 sample + 18 sample)

| 2D | | | | |
|---|---|---|---|---|
| Master# | Protein | Mass | pI | Coverage |
| 44 | complement component C3 | 187046 | 6.02 | 3% |
| 44 | megakaryocyte stimulating factor | 150998 | 9.53 | 3% |
| 44 | PREDICTED: similar to Apolipoprotein(a) | 14584 | 5.79 | 11% |
| | precursor (Apo(a)) (Lp(a)) | | | |
| 44 | PREDICTED: similar to Hornerin | 187937 | 9.82 | 2% |
| 78 | fibronectin precursor | 260064 | 5.45 | 17% |
| 78 | ceruloplasmin | 98321 | 5.29 | 15% |
| 78 | putative | 2269 | 9.97 | 38% |
| 78 | hemopexin | 13452 | 6.70 | 9% |
| 78 | PREDICTED: similar to Apolipoprotein(a) | 14926 | 5.79 | 7% |
| | precursor | | | |
| 78 | development and differentiation enhancing factor- | 100177 | 5.98 | 0% |
| | like 1 | | | |
| 78 | cAMP-specific phosphodiesterase PDE4D5 | 84945 | 5.03 | 1% |
| 94 | fibronectin 1 isoform 3 preproprotein | 262656 | 5.49 | 18% |
| 94 | PREDICTED: similar to Apolipoprotein(a) | 14926 | 5.79 | 17% |
| | precursor (Apo(a)) (Lp(a)) | | | |
| 94 | putative | 2269 | 9.79 | 38% |
| 94 | complement component C3 | 188585 | 6.02 | 1% |
| 94 | dermcidin preproprotein | 11391 | 6.08 | 10% |
| 94 | megakaryocyte stimulating factor; MSF | 152195 | 9.53 | 1% |
| 94 | Synaptonemal complex protein 1 (SCP-1) | 114626 | 5.85 | 3% |
| 94 | creatine kinase M | 43247 | 6.63 | 1% |
| 94 | predicted: similar to ribosomal protein L36 | 5528 | 10.50 | 21% |
| 94 | predicted: similar to cyclin G-associated kinase | 148776 | 9.19 | 4% |
| 94 | cAMP-specific phosphodiesterase PDE4D5 | 84945 | 5.03 | 1% |
| 96 | hypothetical protein | 272166 | 5.30 | 11% |
| 96 | alpha-2-macroglobulin precursor | 163175 | 6.00 | 8% |
| 96 | complement component C3 | 187046 | 6.02 | 2% |
| 96 | dermcidin preproprotein | 11277 | 6.08 | 16% |
| 96 | hornerin precursor | 282199 | 10.04 | 2% |
| 123 | hypothetical protein | 248918 | 5.92 | 11% |
| 123 | lipoprotein | 226369 | 5.71 | 5% |
| 123 | Chain B, Human Complement Component C3 | 112869 | 5.55 | 2% |
| 123 | plasma protease (C1) inhibitor precursor | 55147 | 6.09 | 3% |
| 123 | megakaryocyte stimulating factor | 150998 | 9.53 | 1% |
| 123 | Chain A, Antithrombin Iii | 49008 | 5.95 | 3% |
| 130 | annexin A2 isoform 2 | 38808 | 7.57 | 17% |
| 130 | putative | 2269 | 9.79 | 38% |
| 130 | complement component 3 precursor | 188569 | 6.02 | 7% |
| 130 | plasminogen | 93233 | 7.04 | 5% |
| 130 | fibronectin precursor | 260064 | 5.45 | 0% |
| 130 | dermcidin preproprotein | 11391 | 6.08 | 10% |
| 130 | unnamed protein product | 112600 | 8.27 | 0% |
| 130 | hCG2044987 | 11472 | 9.73 | 14% |
| 162 | alpha-2-macroglobulin | 164600 | 6.00 | 27% |
| 162 | putative | 2269 | 9.79 | 38% |
| 162 | dermcidin preproprotein | 11391 | 6.08 | 10% |
| 162 | annexin A2 isoform 2 | 38808 | 7.57 | 12% |
| 162 | plasminogen | 93263 | 6.89 | 1% |
| 162 | gelsolin isoform a precursor | 86043 | 5.90 | 2% |
| 162 | annexin I | 38918 | 6.57 | 10% |
| 162 | supported by mouse EST AA538043 | 37423 | 9.28 | 2% |
| | (NID: g2284036) | | | |
| 167 | alpha-2-macroglobulin precursor | 164600 | 6.00 | 24% |
| 167 | Putative | 2269 | 9.79 | 38% |
| 167 | filaggrin 2 | 249296 | 8.45 | 1% |
| 167 | dermcidin preproprotein | 11391 | 6.08 | 10% |
| 167 | complement component C3 | 188585 | 6.02 | 0% |
| 167 | Rb1-inducible coiled coil protein | 185051 | 5.31 | 1% |
| 167 | PREDICTED: similar to ribosomal protein L36 | 5528 | 10.50 | 21% |
| | [Pan troglodytes] | | | |
| 167 | cAMP-specific phosphodiesterase PDE4D5 | 84945 | 5.03 | 3% |
| 167 | chromosome 14 open reading frame 104, isoform | 48485 | 9.56 | 2% |
| | CRA_a [Homo sapiens] | | | |
| 167 | CCDC25 protein | 21006 | 8.79 | 5% |
| 167 | KIAA1306 protein | 120084 | 9.36 | 2% |
| 167 | unnamed protein product | 112600 | 8.27 | 0% |
| 167 | annexin II cell-surface form = cytomegalovirus | 2160 | 5.80 | 45% |
| | binding protein | | | |
| 172 | alpha-2-macroglobulin | 164600 | 6.00 | 25% |
| 172 | putative | 2269 | 9.79 | 38% |
| 172 | collagen, type III, alpha 1 preproprotein | 139724 | 6.18 | 3% |
| 172 | dermcidin preproprotein | 11391 | 6.08 | 10% |
| 172 | annexin A2, isoform CRA_c | 32600 | 5.93 | 9% |
| 172 | hCG22067 | 30319 | 7.98 | 3% |
| 172 | cAMP-specific phosphodiesterase PDE4D5 | 84945 | 5.03 | 1% |
| 172 | hypothetical protein LOC400867 | 15522 | 10.07 | 4% |
| 266 | complement factor H | 143694 | 6.21 | 43% |
| 266 | alpha 2 macroglobulin | 167505 | 6.06 | 27% |
| 266 | inter-alpha-trypsin inhibitor heavy chain H2 | 106826 | 6.40 | 14% |
| | precursor | | | |
| 266 | inter-alpha (globulin) inhibitor H1 | 101795 | 6.31 | 15% |
| 266 | fibronectin precursor | 260064 | 5.45 | 3% |
| 266 | chain B, complement component C3 | 114238 | 5.55 | 11% |
| 266 | dermcidin preproprotein | 11391 | 6.08 | 10% |
| 266 | annexin A2 isoform 2 | 38808 | 7.57 | 7% |
| 266 | gelsolin isoform a precursor | 86043 | 5.90 | 5% |
| 266 | hemopexin precursor | 52254 | 6.57 | 7% |
| 266 | cAMP-specific phosphodiesterase PDE4D5 | 84945 | 5.03 | 2% |
| 448 | complement component C3 | 187046 | 6.02 | 22% |
| 448 | alpha-2-macroglobulin precursor | 163175 | 6.00 | 14% |
| 448 | complement component C3b | 25280 | 4.49 | 30% |
| 448 | hemopexin, isoform CRA_a | 22935 | 6.78 | 7% |
| 448 | annexin A2 isoform 2 | 38580 | 7.57 | 7% |
| 569 | complement factor B | 86819 | 6.55 | 34% |
| 569 | Alpha-2-macroglobulin precursor (Alpha-2-M) | 164600 | 6.00 | 18% |
| 569 | complement protein C7 precursor | 96647 | 6.09 | 10% |
| 569 | unnamed protein product | 84549 | 7.23 | 11% |
| 569 | putative | 2269 | 9.79 | 38% |
| 569 | Plasminogen | 93263 | 6.89 | 1% |
| 569 | apg-1 | 95472 | 5.65 | 2% |
| 706 | Inter-alpha-trypsin inhibitor heavy chain H1 | 101782 | 6.31 | 17% |
| | precursor | | | |
| 706 | Coagulation factor XIII A chain precursor | 837228 | 5.75 | 15% |
| | (Coagulation factor XIIIa) (Protein-glutamine | | | |
| | gamma-glutamyltransferase A chain) | | | |
| 706 | fibrinogen gamma chain, isoform CRA_a | 38056 | 5.87 | 32% |
| 706 | putative | 2269 | 9.79 | 38% |
| 706 | glucosamine (N-acetyl)-6-sulfatase precursor | 62840 | 8.60 | 8% |
| 706 | Alpha-2-macroglobulin precursor (Alpha-2-M) | 164600 | 6.00 | 2% |
| 706 | annexin A2 isoform 2 | 38808 | 7.57 | 14% |
| 706 | hornerin precursor | 283111 | 10.04 | 1% |
| 706 | cathepsin D preproprotein | 45037 | 6.10 | 15% |
| 706 | Proapolipoprotein | 28944 | 5.45 | 5% |
| 706 | Coagulation factor XIII B chain precursor | 77723 | 5.97 | 1% |
| | (Protein-glutamine gamma-glutamyltransferase B | | | |
| | chain) (Transglutaminase B chain) (Fibrin- | | | |
| | stabilizing factor B subunit) | | | |
| 706 | hemopexin precursor | 52254 | 6.57 | 4% |
| 706 | cAMP-specific phosphodiesterase PDE4D5 | 84945 | 5.03 | 1% |
| 706 | coagulation factor XII | 68618 | 7.94 | 1% |
| 706 | PREDICTED: similar to ribosomal protein L36 | 5528 | 10.50 | 21% |
| | [Pan troglodytes] | | | |
| 723 | chain A, structure of complement C3b | 71459 | 6.82 | 21% |
| 723 | putative | 2269 | 9.97 | 38% |
| 723 | complement component C4A | 194337 | 6.65 | 4% |
| 723 | coagulation factor XII | 68618 | 7.94 | 6% |
| 723 | annexin A2 isoform 2 | 38808 | 7.57 | 6% |
| 723 | cAMP-specific phosphodiesterase PDE4D5 | 84945 | 5.03 | 1% |
| 752 | inter-alpha (globulin) inhibitor H1 | 101795 | 6.31 | 21% |
| 752 | peptidoglycan recognition protein L precursor | 68669 | 7.62 | 7% |
| 752 | Chain B, Human Complement Component C3 | 114238 | 5.55 | 8% |
| 752 | hemopexin, isoform CRA_d | 43771 | 6.24 | 9% |
| 752 | heparin cofactor II precursor | 57233 | 6.41 | 2% |
| 760 | Chain B, Human Complement Component C3 | 112869 | 5.55 | 47% |
| 760 | inter-alpha (globulin) inhibitor H1 | 101339 | 6.31 | 25% |
| 760 | Inter-alpha-trypsin inhibitor heavy chain H1 | 10326 | 6.31 | 24% |
| | precursor (ITI heavy chain H1) (Inter-alpha- | | | |
| | inhibitor heavy chain 1) (Inter-alpha-trypsin | | | |
| | inhibitor complex component III) (Serum-derived | | | |
| | hyaluronan-associated protein) (SHAP) | | | |
| 760 | Chain A, Crystal Structure Of Native Heparin | 54925 | 6.26 | 32% |
| | Cofactor Ii | | | |
| 760 | peptidoglycan recognition protein L precursor | 67927 | 7.62 | 17% |
| 760 | extracellular matrix protein 1 isoform 1 precursor | 60665 | 6.25 | 22% |
| 760 | complement component 4 binding protein, alpha | 66989 | 7.15 | 13% |
| | chain precursor | | | |
| 760 | glucosamine (N-acetyl)-6-sulfatase precursor | 62042 | 8.60 | 17% |
| 760 | hemopexin precursor | 51512 | 6.57 | 14% |
| 760 | fibrinogen gamma chain | 49450 | 5.61 | 10% |
| 760 | histidine-rich glycoprotein precursor | 59541 | 7.09 | 7% |
| 760 | Dopamine beta-hydroxylase precursor (Dopamine | 67570 | 5.09 | 9% |
| | beta-monooxygenase) | | | |
| 760 | gp180-carboxypeptidase D-like enzyme | 152819 | 5.70 | 3% |
| 760 | Alpha 2 macroglobulin variant | 164030 | 6.00 | 5% |
| 760 | Regucalcin | 33231 | 5.89 | 12% |
| 760 | annexin A2 isoform 2 | 38580 | 7.57 | 9% |
| 760 | complement 8 alpha subunit | 65111 | 6.21 | 9% |
| 760 | glutathione transferase M3 | 26671 | 5.37 | 4% |
| 760 | unnamed protein product | 69250 | 5.92 | 6% |
| 760 | gelsolin isoform a precursor | 85644 | 5.90 | 1% |
| 760 | hypothetical protein | 35102 | 7.31 | 8% |
| 760 | hCG22067 | 29692 | 7.98 | 6% |
| 760 | cAMP-specific phosphodiesterase PDE4D5 | 84375 | 5.03 | 2% |
| 766 | inter-alpha (globulin) inhibitor H1 | 101795 | 6.31 | 25% |
| 766 | inter-alpha-trypsin inhibitor | 101782 | 6.31 | 23% |
| 766 | complement component C3 | 188585 | 6.02 | 22% |
| 766 | extracellular matrix protein (precursor) | 62262 | 6.25 | 29% |
| 766 | alpha-2-macroglobulin precursor | 164600 | 6.00 | 9% |
| 766 | complement component 4 binding protein | 69042 | 7.15 | 16% |
| | (precursor) | | | |
| 766 | dopamine beta-hydroxylase precursor | 68425 | 5.90 | 16% |
| 766 | gp180-carboxypeptidase D-like enzyme | 153903 | 5.70 | 3% |
| 766 | glucosamine (N-acetyl)-6-sulfatase precursor | 62840 | 8.60 | 10% |
| 766 | histidine-rich glycoprotein precursor | 60510 | 7.09 | 12% |
| 766 | annexin A2 isoform 2 | 38808 | 7.57 | 9% |
| 766 | hemopexin precursor | 52254 | 6.57 | 13% |
| 766 | fibrinogen gamma chain | 50077 | 5.61 | 7% |
| 766 | heparin cofactor II precursor | 57233 | 6.41 | 3% |
| 766 | peptidoglycan recognition protein L precursor | 68669 | 7.62 | 5% |
| 766 | regucalcin | 33802 | 5.89 | 5% |
| 766 | glucocerebrosidase precursor | 57798 | 7.03 | 3% |
| 766 | dermcidin preproprotein | 11391 | 6.08 | 10% |
| 766 | unnamed protein | 71246 | 5.92 | 4% |
| 766 | ASC-1 complex subunit P200 | 220646 | 7.23 | 0% |
| 766 | lumican | 38717 | 6.16 | 5% |
| 766 | plasminogen | 93263 | 6.89 | 1% |
| 766 | filaggrin 2 | 249296 | 8.45 | 0% |
| 766 | cAMP-specific phosphodiesterase PDE4D5 | 84945 | 5.03 | 2% |
| 766 | CCDC25 | 21006 | 8.79 | 5% |
| 797 | complement component C3 | 188585 | 6.02 | 19% |
| 797 | chain A, crystal structure of native heparin | 55096 | 6.26 | 41% |
| | cofactor Ii | | | |
| 797 | complement component 4 binding protein | 69042 | 7.15 | 32% |
| 797 | hemopexin precursor | 52254 | 6.75 | 42% |
| 797 | gelsolin isoform a precursor | 86043 | 5.90 | 8% |
| 797 | inter-alpha-trypsin inhibitor C-terminal | 93745 | 6.02 | 5% |
| 797 | peptidoglycan recognition protein L precursor | 68669 | 7.62 | 14% |
| 797 | putative | 2269 | 9.78 | 38% |
| 797 | complement C4B precursor | 189599 | 7.39 | 1% |
| 797 | extracellular matrix protein 1 isoform 1 precursor | 62262 | 6.26 | 6% |
| | [Homo sapiens] | | | |
| 797 | histidine-rich glycoprotein precursor | 60510 | 7.09 | 6% |
| 797 | alpha-2-macroglobulin precursor | 164600 | 6.00 | 3% |
| 797 | ectonucleotide pyrophosphatase/phosphodiesterase | 54745 | 5.94 | 2% |
| | 5 (putative function) | | | |
| 797 | hornerin | 48797 | 9.71 | 3% |
| 797 | Plasminogen | 93263 | 6.89 | 1% |
| 797 | microcephalin | 42749 | 7.65 | 12% |
| 797 | immunoglobulin heavy chain variable region | 12944 | 8.63 | 12% |
| 797 | dimethylarginine dimethylaminohydrolase 1 | 31444 | 5.53 | 3% |
| 797 | cAMP-specific phosphodiesterase PDE4D5 | 84945 | 5.03 | 2% |
| 797 | hypothetical protein LOC400867 | 15522 | 10.07 | 4% |
| 797 | cardiotrophin-like cytokine factor 1 | 25388 | 8.68 | 3% |
| 802 | Chain B, Structure Of Complement C3b: Insights | 104912 | 5.18 | 37% |
| | Into Complement Activation And Regulation | | | |
| 802 | peptidoglycan recognition protein L precursor | 68669 | 7.62 | 12% |
| 802 | hemopexin precursor | 52254 | 6.57 | 20% |
| 802 | complement component 4 binding protein, alpha | 69042 | 7.15 | 7% |
| | chain precursor | | | |
| 802 | Chain A, Crystal Structure Of Native Heparin | 55096 | 6.26 | 13% |
| | Cofactor Ii | | | |
| 802 | putative | 2269 | 9.79 | 38% |
| 803 | Chain B, Structure Of Complement C3b: Insights | 104912 | 5.18 | 36% |
| | Into Complement Activation And Regulation | | | |
| 803 | complement component 4 binding protein, alpha | 69042 | 7.15 | 30% |
| | chain precursor | | | |
| 803 | Chain A, Crystal Structure Of Native Heparin | 55096 | 6.26 | 32% |
| | Cofactor Ii | | | |
| 803 | Alpha-2-macroglobulin precursor (Alpha-2-M) | 164600 | 6.00 | 4% |
| 803 | hemopexin precursor | 52254 | 6.57 | 18% |
| 803 | peptidoglycan recognition protein L precursor | 68669 | 7.62 | 4% |
| 803 | putative [Homo sapiens] | 2269 | 9.79 | 38% |
| 803 | inter-alpha-trypsin inhibitor C-terminal | 93745 | 6.02 | 3% |
| 803 | unnamed protein product | 71246 | 5.92 | 1% |
| 803 | cystic fibrosis transmembrane conductance | 169036 | 8.91 | 0% |
| | regulator | | | |
| 813 | peptidoglycan recognition protein L precursor | 68669 | 7.62 | 8% |
| 813 | unnamed protein product | 60273 | 6.13 | 12% |
| 813 | Putative | 2269 | 9.79 | 38% |
| 813 | Chain A, Crystal Structure Of Native Heparin | 55096 | 6.26 | 13% |
| | Cofactor Ii | | | |
| 813 | hemopexin precursor | 52254 | 6.57 | 12% |
| 813 | complement component 3 precursor | 188569 | 6.02 | 3% |
| 813 | hypothetical protein LOC400867 | 15522 | 10.07 | 4% |
| 813 | cAMP-specific phosphodiesterase PDE4D5 | 84945 | 5.03 | 1% |
| 818 | DEP domain containing 1, isoform | 84786 | 8.71 | 0% |
| 818 | Putative | 2269 | 9.79 | 38% |
| 818 | Mitochondrial ribosomal protein L30 | 18678 | 10.10 | 5% |
| 822 | alpha-1-B-glycoprotein | 52479 | 5.65 | 35% |
| 822 | Chain B, Structure of Complement C3b: . . . | 104912 | 5.18 | 25% |
| 822 | Chain A, Gelatinase A (full length) | 71842 | 5.20 | 20% |
| 822 | C9 complement protein | 64399 | 5.49 | 14% |
| 822 | hemopexin precursor | 52254 | 6.57 | 16% |
| 822 | histidine-rich glycoprotein precursor | 60510 | 7.09 | 9% |
| 822 | complement component 5 variant | 124357 | 8.43 | 3% |
| 822 | Lumican | 38717 | 6.16 | 8% |
| 822 | alpha-2-antiplasmin precursor | 54536 | 5.71 | 10% |
| 822 | complement C4B precursor | 189599 | 7.39 | 2% |
| 822 | vitamin K-dependent protein S precursor | 77127 | 5.48 | 2% |
| 822 | ASPIC | 71448 | 4.98 | 1% |
| 823 | complement component 4 binding protein, alpha | 69042 | 7.15 | 28% |
| | chain precursor | | | |
| 823 | complement component 3 precursor | 188569 | 6.02 | 11% |
| 823 | inter-alpha-trypsin inhibitor C-terminal | 93745 | 6.02 | 7% |
| 823 | peptidoglycan recognition protein L precursor | 68669 | 7.62 | 8% |
| 823 | Putative | 2269 | 9.79 | 38% |
| 823 | extracellular matrix protein 1 isoform 1 precursor | 62262 | 6.25 | 4% |
| 823 | Alpha-2-macroglobulin precursor (Alpha-2-M) | 164600 | 6.00 | 4% |
| 823 | hemopexin precursor | 52254 | 6.57 | 11% |
| 823 | annexin A2 isoform 2 | 38808 | 7.57 | 6% |
| 823 | trypsin inhibitor | 107103 | 6.58 | 2% |
| 823 | dermcidin preproprotein | 11391 | 6.08 | 10% |
| 824 | complement component 4 binding protein, alpha | 66989 | 7.15 | 33% |
| | chain precursor | | | |
| 824 | complement component 3 precursor | 187030 | 6.02 | 13% |
| 824 | N-acetylmuramoyl-L_alanine amidase precursor | 62178 | 7.25 | 18% |
| 824 | inter-alpha-trypsin inhibitor | 93402 | 6.02 | 3% |
| 824 | alpha-2-macroglobulin precursor | 163175 | 6.00 | 6% |
| 824 | annexin A2 isoform 2 | 385800 | 7.57 | 10% |
| 824 | glucocerebrosidase precursor | 57399 | 7.03 | 6% |
| 824 | hemopexin precursor | 51512 | 6.57 | 10% |
| 824 | complement component C3b - human (fragments) | 25280 | 4.49 | 10% |
| 824 | complement protein C7 precursor | 93453 | 6.09 | 2% |
| 824 | filaggrin 2 | 247928 | 8.45 | 1% |
| 869 | chain A, Structure of Complement C3b | 71459 | 6.82 | 65% |
| 869 | putative | 2269 | 9.79 | 38% |
| 869 | DEP domain containing 1, isoform CRA_c | 84786 | 8.71 | 0% |
| 869 | KIAA1481 | 150746 | 6.77 | 1% |
| 956 | hemopexin precursor | 52254 | 6.57 | 22% |
| 956 | alpha-2-macroglobulin precursor | 164600 | 6.00 | 9% |
| 956 | kallistatin | 48696 | 7.33 | 13% |
| 956 | annexin A2 isoform 2 | 38808 | 7.57 | 12% |
| 956 | Putative | 2269 | 9.97 | 38% |
| 956 | filaggrin 2 | 249269 | 8.45 | 0% |
| 956 | dermcidin preproprotein | 11391 | 6.08 | 10% |
| 956 | alpha-fibrinogen precursor | 70223 | 8.26 | 2% |
| 956 | fibronectin precursor | 260064 | 5.45 | 2% |
| 956 | phospholipase D3 isoform 2 | 49196 | 6.00 | 2% |
| 956 | cAMP-specific phosphodiesterase PDE4D5 | 84945 | 5.03 | 3% |
| 956 | Synaptonemal complex protein 1 (SCP-1) | 114626 | 5.85 | 2% |
| 956 | immunoglobulin heavy chain variable region | 12944 | 8.63 | 12% |
| 956 | PREDICTED: similar to mucin 19 | 712778 | 4.82 | 0% |
| 967 | Chain B, Structure of Complement C3b | 103886 | 5.18 | 26% |
| 967 | hemopexin precursor | 51512 | 6.57 | 12% |
| 967 | complement component 8 | 65121 | 6.07 | 9% |
| 967 | alpha-2-antiplasmin precursor | 54194 | 5.71 | 4% |
| 967 | complement C8-beta propeptide | 62008 | 8.24 | 3% |
| 967 | Ceruloplasmin | 97637 | 5.29 | 5% |
| 967 | Regucalcin | 33231 | 5.89 | 5% |
| 967 | Chain I, Crystal Structure of Antithrombin-Iii | 48409 | 5.72 | 9% |
| 967 | trypsin inhibitor | 106647 | 6.58 | 1% |
| 967 | gelsolin isoform a precursor | 85644 | 5.90 | 1% |
| 967 | complement C4B precursor | 188230 | 7.39 | 1% |
| 967 | hyaluron binding protein 2 | 62630 | 6.09 | 1% |
| 967 | afamin precursor | 69024 | 5.64 | 2% |
| 967 | alpha-2-macroglobulin precursor | 163175 | 6.00 | 1% |
| 967 | lipopolysaccharide binding protein precursor | 52912 | 6.23 | 3% |
| 967 | annexin A2 isoform 2 | 38580 | 7.57 | 9% |
| 967 | unnamed protein product | 66412 | 5.62 | 8% |
| 967 | alpha 2-plasmin inhibitor, alpha 2-PI {N-terminal, | 2064 | 4.53 | 63% |
| | form A} [human, plasma, Peptide Partial, 19 aa] | | | |
| 967 | fibrinogen | 49450 | 5.61 | 2% |
| 967 | hCG22067 | 29692 | 7.98 | 6% |
| 967 | beta-2-glycoprotein | 38273 | 8.34 | 2% |
| 1400 | putative | 2269 | 9.79 | 38% |
| 1400 | complement component C3 | 188585 | 6.02 | 1% |
| 1400 | Serum paraoxonase/arylesterase 1 (PON 1) | 39895 | 5.08 | 6% |
| | (Serum aryldialkylphosphatase 1) (A-esterase 1) | | | |
| | (Aromatic esterase 1) (K-45) | | | |
| 1400 | dermcidin preproprotein | 11391 | 6.08 | 10% |
| 1400 | hypothetical protein LOC400867 | 15522 | 10.07 | 4% |
| 1400 | cAMP-specific phosphodiesterase PDE4D5 | 84945 | 5.03 | 3% |
| 1400 | PREDICTED: hypothetical protein | 26918 | 11.95 | 2% |
| 1400 | PREDICTED: similar to ribosomal protein L36 | 5528 | 10.50 | 21% |
| | [Pan troglodytes] | | | |
| 1400 | Rho guanine nucleotide exchange factor (GEF) 17 | 223645 | 5.90 | 0% |
| 1417 | Serum paraoxonase/arylesterase 1 (PON 1) | 66170 | 5.08 | 22% |
| | (Serum aryldialkylphosphatase 1) (A-esterase 1) | | | |
| | (Aromatic esterase 1) (K-45) | | | |
| 1417 | Putative | 2269 | 9.79 | 38% |
| 1417 | complement C4B precursor | 189599 | 7.39 | 3% |
| 1417 | apolipoprotein A-IV precursor | 45353 | 5.33 | 9% |
| 1417 | mutant beta-actin (beta′-actin) | 42128 | 5.22 | 12% |
| 1417 | complement component C3 | 188585 | 6.02 | 1% |
| 1417 | small proline-rich protein | 8676 | 9.07 | 12% |
| 1417 | Plasminogen | 93263 | 6.89 | 1% |
| 1417 | Chain A, Hr1b Domain From Prk1 | 9054 | 9.77 | 11% |
| 1417 | coiled-coil domain containing 96 | 62958 | 4.92 | 1% |
| 1417 | cAMP-specific phosphodiesterase PDE4D5 | 84945 | 5.03 | 1% |
| 27 | alpha-1-antitrypsin | 46848 | 5.43 | 25% |
| 27 | alpha-2-macroglobulin precursor | 164,600 | 6 | 9% |
| 27 | dermcidin preproprotein | 11391 | 6.08 | 1% |
| 27 | megakaryocyte stimulating factor | 152195 | 9.53 | 1% |
| 27 | cAMP-specific phosphodiesterase | 84945 | 5.03 | 1% |
| 76 | fibronectin precursor | 260064 | 5.45 | 7% |
| 76 | Chain B, Structure of Complement C3b | 104912 | 5.18 | 12% |
| 76 | Chain A, Thyroxine-Binding Globulin Complex | 42680 | 5.69 | 17% |
| | With Throxine | | | |
| 76 | filaggrin | 249296 | 8.45 | 0% |
| 76 | C9 complement protein | 64399 | 5.49 | 3% |
| 76 | afamin | 70963 | 5.64 | 6% |
| 76 | Chain, Intact Recombined Alpha-1-Antitrypsin | 44322 | 5.43 | 16% |
| | Mutant Phe 51 to Leu | | | |
| 76 | C4A3 | 58960 | 5.67 | 5% |
| 76 | lipoprotein, Lp(a) | 134467 | 5.77 | 1% |
| 76 | hemopexin | 52254 | 6.57 | 4% |
| 76 | plasminogen | 93263 | 6.89 | 1% |
| 76 | Chain A, Hemoglobulin Thionville Alpha Chain | 15446 | 7.82 | 12% |
| | Mutant | | | |
| 76 | Chain A, Hr1b Domain from Prk1 | 9054 | 9.77 | 11% |
| 93 | Chain B, Crystal Structure of S-Nitroso-Nitrosyl | 15922 | 6.81 | 23% |
| | Human Hemoglobin | | | |
| 93 | alpha-1-antitrypsin | 46848 | 5.43 | 12% |
| 93 | megakaryocyte stimulating factor | 152195 | 9.53 | 1% |
| 124 | kininogen 1 | 48936 | 6.29 | 31% |
| 124 | fibronectin precursor | 260064 | 5.45 | 4% |
| 124 | unnamed protein, coagulation factor II precursor | 70723 | 5.53 | 7% |
| 206 (—) | alpha-1-antichymotrypsin | 48834 | 5.79 | 9% |
| 206 (—) | alpha-1-antitrypsin | 13859 | 7.93 | 21% |
| 206 (—) | aggrecan core protein precursor | 251979 | 4.1 | 0% |
| 206 (—) | plasminogen | 93263 | 6.89 | 1% |
| 206 (—) | creatine kinase M | 43247 | 6.63 | 3% |
| 206 (—) | secretoglobin, family 3A | 10269 | 6.71 | 9% |
| 210 | alpha-2-macroglobulin | 167505 | 6.06 | 23% |
| 210 | complement component C3 | 188585 | 6.02 | 11% |
| 210 | complement factor H | 143694 | 6.21 | 6% |
| 210 | hemopexin precursor | 52254 | 6.57 | 8% |
| 210 | annexin A2 | 38808 | 7.57 | 3% |
| 246 | alpha 2 macroglobulin | 167505 | 6.06 | 15% |
| 246 | complement factor H | 143694 | 6.21 | 21% |
| 246 | complement component C3 | 188585 | 6.02 | 4% |
| 255 | macroglobulin alpha2 | 162072 | 5.95 | 17% |
| 255 | complement factor H | 143694 | 6.21 | 15% |
| 265 | cAMP-specific phosphodiesterase | 2735857 | 6.93 | 4% |
| 292 | alpha-2-macroglobulin precursor | 164600 | 6 | 19% |
| 292 | complement factor H | 143694 | 6.21 | 19% |
| 292 | trypsin inhibitor | 107103 | 6.58 | 8% |
| 292 | inter-alpha-trypsin heavy chain H1 precursor | 101782 | 6.31 | 9% |
| 292 | gelsolin isoform a precursor | 86043 | 5.9 | 1% |
| 292 | cAMP-specific phosphodiesterase | 84945 | 5.03 | 4% |
| 372 | alpha 2 macroglobulin | 167505 | 6.06 | 17% |
| 372 | complement factor H isoform a precursor | 143654 | 6.23 | 4% |
| 372 | gelsolin isoform a precursor | 86043 | 5.9 | 6% |
| 372 | hemopexin precursor | 52254 | 6.57 | 4% |
| 384 | ceruloplasmin | 116197 | 5.43 | 14% |
| 384 | inter-alpha-trypsin inhibitor heavy chain H4 | 103489 | 6.51 | 16% |
| | precursor | | | |
| 384 | phosphatidylinositol-glycan-specific | 92943 | 5.91 | 12% |
| | phospholipase D1 precursor | | | |
| 384 | complement factor H isoform a precursor | 143654 | 6.23 | 2% |
| 384 | alpha-2-macroglobulin precursor | 164600 | 6 | 3% |
| 385 | phosphatidylinositol-glycan-specific | 92943 | 5.91 | 18% |
| | phospholipase D1 precursor | | | |
| 385 | ceruloplasmin | 116197 | 5.43 | 11% |
| 385 | inter-alpha-trypsin inhibitor family heavy chain- | 103536 | 6.64 | 8% |
| | related protein | | | |
| 385 | alpha-2-macroglobulin precursor | 164600 | 6 | 3% |
| 385 | kininogen | 48936 | 6.29 | 6% |
| 385 | complement C4B | 189599 | 7.39 | 3% |
| 385 | complement component C3 | 188585 | 6.02 | 1% |
| 393 | ceruloplasmin | 116197 | 5.43 | 11% |
| 393 | inter-alpha-trypsin inhibitor heavy chain H4 | 103489 | 6.51 | 17% |
| | precursor | | | |
| 393 | phosphatidylinositol-glycan-specific | 92943 | 5.91 | 7% |
| | phospholipase D1 precursor | | | |
| 393 | alpha-2-macroglobulin precursor | 164600 | 6 | 2% |
| 393 | annexin A2 isoform 2 | 38808 | 7.57 | 6% |
| 393 | complement component C3 | 188585 | 6.02 | 1% |
| 393 | factor H | 143710 | 6.28 | 1% |
| 394 | inter-alpha-trypsin inhibitor heavy chain H4 | 103489 | 6.51 | 16% |
| | precursor | | | |
| 394 | ceruloplasmin | 116197 | 5.43 | 18% |
| 394 | hornerin | 48797 | 9.71 | 6% |
| 394 | cAMP-specific phosphodiesterase | 84945 | 5.03 | 2% |
| 394 | factor H | 143710 | 6.28 | 0% |
| 394 | creatine kinase M | 43247 | 6.63 | 3% |
| 408 | glycosylphosphatidylinositol specific phosphatase | 93899 | 5.96 | 13% |
| | D1 | | | |
| 408 | ceruloplasmin | 116197 | 5.43 | 5% |
| 408 | brain-expressed protein BEX1 | 38717 | 6.31 | 12% |
| 408 | lumican | 38717 | 6.16 | 2% |
| 408 | cAMP-specific phosphodiesterase PDE4D5 | 84945 | 5.03 | 5% |
| 477 | alpha-2-macroglobulin precursor | 163175 | 6 | 22% |
| 477 | complement component C6 precursor peptide | 104718 | 6.39 | 22% |
| 477 | complement factor B | 85450 | 6.55 | 11% |
| 477 | inter-alpha-trypsin inhibitor family heavy chain- | 103321 | 6.51 | 2% |
| | related protein | | | |
| 477 | ceruloplasmin | 115398 | 5.43 | 4% |
| 477 | annexin A2 isoform 2 | 38580 | 7.57 | 7% |
| 481 (—) | pre-pro-alpha (I) collagen | 138827 | 5.66 | 10% |
| 495 | inter-alpha-trypsin inhibitor family heavy chain- | 103321 | 6.51 | 20% |
| | related protein | | | |
| 495 | ceruloplasmin | 115398 | 5.43 | 14% |
| 495 | complement component C3 | 187046 | 6.02 | 10% |
| 495 | macroglobulin alpha2 | 160704 | 5.95 | 11% |
| 495 | complement C4B precursor | 188230 | 7.39 | 5% |
| 495 | factor H | 139034 | 6.28 | 6% |
| 495 | SERPINF2 protein | 54559 | 5.87 | 11% |
| 495 | gp-180-carboxypeptidase D-like enzyme | 152819 | 5.7 | 5% |
| 495 | oxidized protein hydrolase | 81201 | 5.29 | 2% |
| 495 | complement component C6 precursor peptide | 104718 | 6.39 | 9% |
| 496 | inter-alpha-trypsin inhibitor family heavy chain- | 103321 | 6.51 | 23% |
| | related protein | | | |
| 496 | Chain B, Human Complement Component C3 | 112869 | 5.55 | 18% |
| 496 | alpha-2-macroglobulin precursor | 163175 | 6 | 13% |
| 496 | ceruloplasmin | 97637 | 5.29 | 11% |
| 496 | complement C4B precursor | 188230 | 7.39 | 7% |
| 496 | complement factor H | 139019 | 6.21 | 11% |
| 496 | complement component C3b | 25280 | 4.49 | 23% |
| 496 | pregnancy-zone protein | 163733 | 5.97 | 6% |
| 644 ? | fibulin-1 isoform D precursor | 77223 | 5.11 | 23% |
| 644 | inter-alpha-trypsin inhibitor family heavy chain- | 103321 | 6.51 | 18% |
| | related protein | | | |
| 644 | plasminogen | 90496 | 7.04 | 15% |
| 644 | complement factor B | 85450 | 6.55 | 15% |
| 644 | HGF activator, preproprotein | 70602 | 6.99 | 14% |
| 644 | lumican | 38375 | 6.16 | 26% |
| 644 | complement component C3 | 187046 | 6.02 | 3% |
| 644 | inter-alpha (globulin) inhibitor H1 | 99357 | 6.59 | 5% |
| 644 | aggrecan core protein precursor | 250040 | 4.1 | 1% |
| 644 | complement protein C7 precursor | 93453 | 6.09 | 2% |
| 644 | alpha-2-macroglobulin precursor | 163175 | 6 | 7% |
| 644 | fibrinogen gamma chain | 49450 | 5.61 | 4% |
| 644 | serpin peptidase inhibitor, clade G | 21826 | 6.06 | 10% |
| 644 | trypsin inhibitor | 106647 | 6.58 | 5% |
| 644 | histidine-rich glycoprotein precursor | 59541 | 7.09 | 4% |
| 746 | trypsin inhibitor | 107103 | 6.58 | 8% |
| 746 | unnamed protein (coagulation factor II precursor) | 70723 | 5.64 | 17% |
| 746 | afamin precursor | 70963 | 5.64 | 16% |
| 746 | Vitamin K-dependent | 77127 | 5.48 | 13% |
| 746 | complement component 1, s subcomponent | 78174 | 4.86 | 15% |
| 746 | inter-alpha-trypsin inhibitor | 99401 | 5.61 | 9% |
| 746 | ASPIC | 71448 | 4.98 | 6% |
| 746 | lumican | 38717 | 6.16 | 21% |
| 746 | inter-alpha (globulin) inhibitor H4 | 101521 | 6.21 | 8% |
| 746 | gelsolin isoform a precursor | 86043 | 5.9 | 6% |
| 746 | phospholipid transfer protein isoform a precursor | 54933 | 6.53 | 6% |
| 746 | lysosomal membrane glycoprotein-2 | 45374 | 5.47 | 1% |
| 746 | alpha-1-B-glycoprotein | 52479 | 5.65 | 6% |
| 746 | creatine kinase M | 43247 | 6.63 | 3% |
| 746 | Chain A, Hr1b Domain From Prk1 | 9054 | 9.77 | 11% |
| 805 | complement component 3 precursor | 188569 | 6.02 | 13% |
| 805 | plasma kallikrein precursor | 73433 | 8.6 | 17% |
| 805 | annexin A2 isoform 2 | 38808 | 7.57 | 24% |
| 805 | complement C4B precursor | 189599 | 7.39 | 3% |
| 805 | gelsolin | 52511 | 5.21 | 6% |
| 805 | Chain, Annexin I | 35246 | 7.77 | 9% |
| 840 | inter-alpha-trypsin inhibitor heavy chain H1 | 101782 | 6.31 | 11% |
| | precursor | | | |
| 840 | glucosamine (N-acetyl)-6-sulfatase precursor | 62840 | 8.6 | 16% |
| 840 | cystic fibrosis antigen (unnamed protein) | 10988 | 9.19 | 31% |
| 840 | alpha 2 macroglobulin | 167505 | 6.06 | 4% |
| 840 | beta-globin | 19204 | 6.28 | 17% |
| 840 | annexin A2 isoform | 38808 | 7.57 | 6% |
| 840 | phospholipase D3 isoform 2 | 49196 | 6 | 8% |
| 840 | Chain A, Crystal Structure of Human Galectin-7 | 14992 | 7 | 14% |
| | In Complex With Galactosamine | | | |
| 840 | ezrin (p81) (cytovillin)(Villin-2) | 69470 | 5.94 | 7% |
| 840 | actin, alpha 1 skeletal muscle | 32370 | 5.26 | 5% |
| 840 | SCC antigen | 44564 | 6.35 | 6% |
| 840 | histidine-rich glycoprotein precursor | 60510 | 7.09 | 1% |
| 840 | S100 Calcium binding protein A9 | 13291 | 5.71 | 17% |
| 840 | hemopexin | 13452 | 6.7 | 9% |
| 849 | inter-alpha-trypsin inhibitor | 101782 | 6.31 | 12% |
| 849 | fibrinogen gamma | 46823 | 5.54 | 25% |
| 849 | glucosamine (N-acetyl)-6-sulfatase precursor | 62840 | 8.6 | 9% |
| 849 | extracellular matrix protein 1 isoform | 62262 | 6.25 | 4% |
| 849 | coagulation factor XIII A chain precursor | 83728 | 5.75 | 7% |
| 849 | alpha 2 macroglobulin | 167505 | 6.06 | 4% |
| 849 | Chain B, Structure of Complement C3b | 104912 | 5.18 | 95 |
| 849 | annexin A2 isoform 2 | 38808 | 7.57 | 9% |
| 849 | beta-globin | 19204 | 6.28 | 7% |
| 849 | hemopexin | 52254 | 6.57 | 4% |
| 849 | peptidoglycan recognition protein L precursor | 68699 | 7.62 | 2% |
| 849 | complement component 4 binding protein | 69052 | 7.62 | 2% |
| 849 | plasminogen | 93263 | 6.89 | 1% |
| 849 | cAMP-specific phosphodiesterase | 84945 | 5.03 | 1% |
| 856 | inter-alpha-trypsin inhibitor heavy chain H1 | 101782 | 6.31 | 13% |
| | precursor | | | |
| 856 | glucosamine (N-acetyl)-6-sulfatase precursor | 62840 | 8.6 | 11% |
| 856 | fibrinogen gamma chain | 50077 | 5.61 | 13% |
| 856 | macroglobulin alpha2 | 162072 | 5.95 | 6% |
| 856 | Coagulation factor XIII A chain precursor | 83728 | 5.75 | 8% |
| 856 | Chain B, T-To-T (High) Quaternary Transitions . . . | 15954 | 6.79 | 36% |
| 856 | annexin A2 isoform | 38808 | 7.57 | 13% |
| 856 | phospholipase D3 | 49196 | 6 | 2% |
| 856 | glucocerebrosidase precursor | 57798 | 7.03 | 4% |
| 856 | histidine-rich glycoprotein precursor | 60510 | 7.09 | 3% |
| 856 | complement component C3 | 188585 | 6.02 | 1% |
| 856 | hemopexin | 13452 | 6.7 | 9% |
| 864 | inter-alpha-trypsin inhibitor heavy chain H1 | 101782 | 6.31 | 9% |
| | precursor | | | |
| 864 | glucosamine (N-acetyl)-6-sulfatase precursor | 62840 | 8.6 | 12% |
| 864 | Ig mu chain precursor | 69208 | 5.82 | 13% |
| 864 | alpha 2 macroglobulin | 167505 | 6.06 | 3% |
| 864 | Chain B, T-To-T (High) . . . | 15975 | 6.75 | 23% |
| 864 | annexin A2 isoform 2 | 38808 | 7.57 | 8% |
| 864 | complement component C3 | 188585 | 6.02 | 1% |
| 864 | complement factor B | 86819 | 6.55 | 2% |
| 864 | phospholipase D3 isoform 2 | 49196 | 6 | 2% |
| 864 | hemopexin | 13452 | 6.7 | 9% |
| 864 | moesin | 67892 | 6.08 | 4% |
| 864 | alpha-2-antiplasmin precursor | 54536 | 5.71 | 4% |
| 962 | complement 9 | 61728 | 5.42 | 31% |
| 962 | Chain B, Structure of Complement C3b | 104912 | 5.18 | 24% |
| 962 | complement C5 precursor | 189923 | 6.11 | 7% |
| 962 | alpha-2-antiplasmin precursor | 54536 | 5.71 | 20% |
| 962 | Chain A, Antithrombin Iii | 49350 | 5.95 | 19% |
| 962 | lumican | 38717 | 6.16 | 19% |
| 962 | kininogen | 48936 | 6.29 | 20% |
| 962 | complement C4B precursor | 189599 | 7.39 | 3% |
| 962 | ceruloplasmin | 116197 | 5.43 | 4% |
| 962 | complement component 1 | 54192 | 6.96 | 12% |
| 962 | hemopexin precursor | 52254 | 6.57 | 9% |
| 962 | alpha 2-plasmin inhibitor | 2064 | 4.53 | 63% |
| 962 | thyroxine-binding globulin precursor | 46637 | 5.87 | 5% |
| 962 | thrombin inhibitor | 42901 | 5.33 | 2% |
| 962 | creatine kinase M | 43247 | 6.63 | 3% |
| 973 | complement 9 | 61728 | 5.42 | 32% |
| 973 | Chain A, antithrombin Iii | 49350 | 5.95 | 35% |
| 973 | alpha-2-antiplasmin precursor | 54536 | 5.71 | 14% |
| 973 | complement component C3 | 188585 | 6.02 | 11% |
| 973 | ceruloplasmin | 116197 | 5.43 | 6% |
| 973 | complement component 5 variant | 124357 | 8.43 | 9% |
| 973 | kinninogen 1 | 48936 | 6.29 | 14% |
| 973 | complement C4B precursor | 189599 | 7.39 | 3% |
| 973 | thrombin inhibitor | 42901 | 5.33 | 7% |
| 973 | L-plastin | 70815 | 5.2 | 12% |
| 973 | complement component 1 | 54192 | 6.75 | 2% |
| 973 | angiotensinogen | 53407 | 5.78 | 2% |
| 973 | alpha 2-plasmin inhibitor | 2064 | 4.53 | 63% |
| 973 | alpha-1-B-glycoprotein | 52479 | 5.65 | 5% |
| 973 | serine (or cysteine) proteinase inhibitor | 43004 | 5.61 | 5% |
| 973 | hemopexin precursor | 52254 | 6.57 | 10% |
| 973 | lumican | 38717 | 6.16 | 12% |
| 973 | proapolipoprotein | 28944 | 5.45 | 5% |
| 973 | phospholipase D3 isoform 2 | 49196 | 6 | 2% |
| 973 | hyaluronan binding protein 2 | 64740 | 6.09 | 1% |
| 973 | cAMP-specific phosphodiesterase | 84945 | 5.03 | 5% |
| 1472 | complement component C4A | 194337 | 6.65 | 5% |
| 1472 | complement component C3 | 188585 | 6.02 | 4% |
| 1472 | hemopexin precursor | 52254 | 6.57 | 18% |
| 1472 | apolipoprotein A-IV precursor | 45353 | 5.33 | 21% |
| 1472 | annexin A2 isoform 2 | 38808 | 7.57 | 6% |
| 1472 | serum paraoxonase/arylesterase | 39895 | 5.08 | 14% |
| 1472 | preprohaptoglobulin | 38941 | 6.13 | 9% |
| 1472 | beta actin | 42052 | 5.29 | 13% |
| 1472 | COMP | 85403 | 4.34 | 1% |
| 1472 | alpha-1-acid glycoprotein 1 precursor | 21433 | 5.09 | 4% |
| 1472 | Zn-alpha2-glycoprotein | 34942 | 5.71 | 3% |
| 1472 | serpin peptidase inhibitor, clade I | 46287 | 5.01 | 4% |
| 1472 | thrombospondin-4 | 108415 | 4.44 | 1% |
| 1472 | coiled-coil domain containing 96 | 62958 | 4.92 | 1% |
| 1472 | Chain B, Cathepsin | 26457 | 5.31 | 2% |
| 1472 | desmoglein-1 precursor | 114670 | 4.9 | 1% |
| 1472 | follistatin-like 1 precursor | 36103 | 5.39 | 3% |
| 1498 | complement factor H-related protein 1 precursor | 38777 | 7.75 | 29% |
| 1498 | Chain B, alpha-Ferrous-Carbonmonoxy | 15971 | 6.81 | 43% |
| 1498 | Chain A, Crystal Structure of Lipid-Free human | 28061 | 5.27 | 35% |
| | Apolipoprotein A-I | | | |
| 1498 | alpha-fibrinogen precursor | 70223 | 8.26 | 9% |
| 1498 | annexin A2 isoform 2 | 38808 | 7.57 | 10% |
| 1498 | lactoferrin (unnamed protein) | 80242 | 8.5 | 2% |
| 1498 | phospholipase D3 isoform 2 | 49146 | 6 | 2% |
| 1498 | Chain B, Cathepsin D | 26457 | 5.31 | 7% |
| 1498 | complement C4B precursor | 189599 | 7.39 | 6% |
| 1498 | glutathione transferase M3 | 27127 | 5.37 | 4% |
| 1498 | Chain E, Structure of human transferrin receptor- | 39476 | 6.41 | 5% |
| | transferrin complex | | | |
| 1498 | complement component C3 | 188585 | 6.02 | 0% |
| 1498 | plasminogen | 93263 | 6.89 | 2% |
| 1498 | neuropolypeptide h3 | 21027 | 7.42 | 13% |
| 1611 | complement factor H-related 1 | 38766 | 7.38 | 35% |
| 1611 | annexin A2 isoform 2 | 38808 | 7.57 | 22% |
| 1611 | Chain B, alpha-Ferrous-Carbonmonoxy | 15971 | 6.81 | 40% |
| 1611 | phospholipase D3 isoform 2 | 49196 | 6 | 2% |
| 1611 | proapolipoprotein | 28944 | 5.45 | 25% |
| 1611 | Chain B, Cathepsin D | 26457 | 5.31 | 7% |
| 1611 | cAMP-specific phosphodiesterase | 84945 | 5.03 | 2% |
| 1611 | DF4 | 10966 | 10.3 | 5% |
| 1867 | complex-forming glycoprotein HC | 20592 | 5.84 | 17% |
| 1867 | dermcidin preproprotein | 11391 | 6.08 | 10% |
| 1867 | protein disulfide isomerase-related protein | 46512 | 4.95 | 2% |
| 1867 | complement factor B | 86819 | 6.55 | 0% |
| 1867 | albumin, isoform CRA_a | 25732 | 6.45 | 3% |
| 1867 | thyroid receptor interactor | 45785 | 7.7 | 8% |
| 1867 | cAMP-specific phosphodiesterase | 84945 | 5.03 | 1% |
| 1956 | annexin A2, isoform CRA_c | 32600 | 5.93 | 24% |
| 1956 | Chain, Carbonic Anhydrase Form B | 28903 | 6.44 | 11% |
| 1956 | Chain B, Alpha-Ferrous-Carbonmonoxy, . . . | 15971 | 6.81 | 23% |
| 1956 | complement Factor H-related Protein 2 | 28706 | 6.52 | 18% |
| 1956 | UCC1 protein (mammalian ependymin-related | 20162 | 5.03 | 6% |
| | protein 1) | | | |
| 1956 | Chain B, Cathepsin D | 26457 | 5.31 | 4% |
| 1956 | RNA polymerase transcriptional regulation | 28505 | 8.91 | 2% |
| | mediator | | | |
| 1956 | serum albumin (unnamed protein) | 71246 | 5.92 | 2% |
| 1956 | cathepsin F | 38244 | 6.54 | 2% |
| 1956 | cAMP-specific phosphodiesterase | 84945 | 5.03 | 1% |
| 2062 | dermcidin preproprotein | 11391 | 6.08 | 10% |
| 2062 | glutathione S-transferase | 25847 | 6.9 | 13% |
| 2062 | mutant beta-globin | 12635 | 5.9 | 20% |
| 2062 | proapolipoprotein | 28944 | 5.45 | 9% |
| 2062 | liprin-beta2 | 88864 | 6.29 | 0% |
| 2062 | HSPC336 (apolipoprotein M) | 21220 | 6.48 | 8% |
| 2062 | cAMP-specific phosphodiesterase | 84945 | 5.03 | 1% |
| 2175 | annexin A2 isoform 2 | 38808 | 7.57 | 6% |
| 2175 | glutathione transferase M3 | 27127 | 5.37 | 4% |
| 2175 | regucalcin | 33802 | 5.89 | 3% |
| 2175 | Chain A, Human Aspartylglucosaminidase | 17552 | 4.82 | 4% |
| 2175 | serum albumin (unnamed protein) | 71246 | 5.92 | 1% |
| 2175 | cAMP-specific phosphodiesterase | 84945 | 5.03 | 1% |
| 2175 | polymerase (DNA directed) | 64070 | 7.96 | 1% |
| 2205 | glia maturation factor, beta | 16874 | 5.19 | 34% |
| 2205 | serum albumin (unnamed protein) | 71246 | 5.92 | 3% |
| 2205 | plasminogen | 93263 | 6.89 | 2% |
| 2205 | nuclear distribution gene C homolog | 38276 | 5.27 | 9% |
| 2205 | Chain A, Hr1b Domain From Prk1 | 9054 | 9.77 | 11% |
| 2205 | glia maturation factor, gamma | 16961 | 5.18 | 18% |
| 2205 | dermcidin preproprotein | 11391 | 6.08 | 10% |
| 1787 | Chain, Human Annexin V with Proline | 36041 | 4.94 | 26% |
| | Substitution by Thioproline | | | |
| 1787 | annexin A2 isoform 2 | 38808 | 7.57 | 10% |
| 1787 | cathepsin Z precursor | 34544 | 6.7 | 3% |
| 1787 | complex-forming glycoprotein HC | 20592 | 5.84 | 7% |
| 1787 | apolipoproprotein J precursor | 49342 | 6.27 | 1% |
| 1787 | apolipoprotein D | 28317 | 5.14 | 4% |
| 1787 | dermcidin preproprotein | 11391 | 6.08 | 10% |
| 1787 | bromodomain adjacent to zinc finger domain 2B | 222440 | 5.95 | 0% |
| 1694 | apolipoprotein J precursor | 49342 | 6.27 | 14% |
| 1694 | apolipoprotein D | 28317 | 5.14 | 4% |
| 1694 | COMP | 855403 | 4.34 | 2% |
| 1694 | annexin A2 isoform 2 | 38808 | 7.57 | 2% |
| 1694 | V-set and immunoglobulin domain containing 4 | 44529 | 5.93 | 2% |
| 1694 | prepro-C3b/C4B | 68120 | 7.72 | 1% |
| 1694 | creatine kinase M | 43247 | 6.63 | 1% |
| 1694 | cAMP-specific phosphodiesterase | 84945 | 5.03 | 1% |
| 1694 | regucalcin | 33802 | 5.89 | 9% |
| 1687 | prepro-C3b/C4B | 68120 | 7.72 | 4% |
| 1687 | regucalcin | 33802 | 5.89 | 12% |
| 1687 | apolipprotein J precursor | 49342 | 6.27 | 11% |
| 1687 | annexin A2 isoform 2 | 38808 | 7.57 | 7% |
| 1687 | ectonucleotide pyrophosphatase/phosphodiesterase | 54745 | 5.94 | 1% |
| 1687 | apolipoprotein E | 36302 | 5.65 | 2% |
| 1687 | Vitamin D-binding protein precursor | 54526 | 5.4 | 2% |
| 1687 | dermcidin preproprotein | 11391 | 6.08 | 10% |
| 1687 | plasminogen | 93263 | 6.89 | 2% |
| 1687 | protein C | 41122 | 6.41 | 3% |
| 1228 | alpha-2 macroglobulin precursor | 164600 | 6 | 6% |
| 1228 | alpha-2-antiplasmin precursor | 54536 | 5.71 | 2% |
| 1228 | annexin A2 isoform 2 | 38808 | 7.57 | 6% |
| 1228 | phospholipase D3 isoform 2 | 49196 | 6 | 2% |
| 1228 | prepro-C3b/C4B | 68120 | 7.72 | 7% |
| 1228 | complement component C3 | 188585 | 6.02 | 2% |
| 1228 | beta globin chain | 11537 | 5.9 | 40% |
| 1228 | prolylcarboxypeptidase isoform 1 preproprotein | 56277 | 6.75 | 3% |
| 1228 | beta-fibrinogen precursor | 55545 | 8.31 | 4% |
| 1228 | Rho guanine nucleotide exchange factor | 223645 | 5.9 | 0% |
| 1228 | cAMP-specific phosphodiesterase | 84945 | 5.03 | 2% |
| 1228 | MEN1 protein | 64077 | 6.19 | 2% |
| 243 | ceruloplasmin | 98321 | 5.29 | 8% |
| 243 | factor H | 143710 | 6.28 | 3% |
| 243 | Vitamin D-binding protein precursor | 54526 | 5.4 | 6% |
| 243 | trypsin inhibitor | 107103 | 6.58 | 1% |
| 243 | complement component C3 | 188585 | 6.02 | 2% |
| 243 | Chain I, Crystal Structure of P13 Alanine Variant | 49276 | 6.13 | 8% |
| | of Antithrombin | | | |
| 243 | complement C4B precursor | 189599 | 7.39 | 4% |
| 243 | cAMP-specific phosphodiesterase | 84945 | 5.03 | 1% |

TABLE 3

| | |
|---|---|
| 44 | fibronectin precursor, complement component C3, alpha-2-macroglobulin |
| | precursor |
| 82 | Chain B, Structure of Complement C3b |
| 109 | factor H, fibronectin precursor |
| 126 | fibronectin 1 isoform 3 preproprotein, alpha-2-macroglobulin precursor, |
| 164 | macroglobulin alpha 2 |
| 184 | alpha-2-macroglobulin |
| 191 | alpha-2-macroglobulin, complement component C3 |
| 205 | no protein matches criteria |
| 216 | collagen type IV alpha 1, alpha 2 type IV collagen preproprotein |
| 244 | collagen type IV alpha 1, alpha 2 type IV collagen preproprotein |
| 252 | alpha 2 macroglobulin, complement factor H, complement component C3 |
| 267 | inter-alpha-trypsin inhibitor, Human Factor H, fibronectin precursor, inter-alpha- |
| | trypsin inhibitor, C-terminal |
| 295 | complement factor H, alpha 2 macroglobulin, trypsin inhibitor, inter-alpha-trypsin |
| | inhibitor |
| 352 | complement factor H, alpha 2 macroglobulin, inter-alpha-trypsin inhibitor, trypsin |
| | inhibitor, complement component C3 |
| 392 | Ceruloplasmin, phosphatidylinositol-glycan-specific phospholipase D1 |
| 396 | afamin precursor, ceruloplasmin, inter-alpha-trypsin inhibitor |
| 469 | complement component 6, isoform CRA_b |
| 509 | inter-alpha-trypsin inhibitor heavy chain-related protein, ceruloplasmin, Chain B, |
| | Structure of Complement C3b |
| 510 | ceruloplasmin (ferroxidase), Chain A, Crystal Structure of human Galectin-7 |
| 512 | ceruloplasmin (ferroxidase), inter-alpha-trypsin inhibitor family heavy chain- |
| | related protein |
| 547 | serum albumin, inter-alpha-trypsin inhibitor family heavy chain-related protein |
| 533 | COMP, ceruloplasmin |
| 554 | inter-alpha-trypsin inhibitor family heavy chain-related protein |
| 555 | ALB protein, gelsolin isoform a precursor |
| 561 | serum albumin, trypsin inhibitor, complement component C3 |
| 567 | Chain B, Human complement component C3 |
| 568 | complement component C3, complement factor B, gelsolin isoform precursor |
| 655 | Ceruloplasmin (ferroxidase), fibulin-1 isoform D precursor, hypothetical protein |
| | (inter-alpha-globulin inhibitor H4), valosin-containing protein, Vitamin D-binding |
| | protein precursor |
| 677 | unnamed protein (complement component 2 precursor), annexin A2, complement |
| | factor B |
| 701 | Annexin A2 isoform 2 |
| 703 | inter-alpha-trypsin inhibitor family heavy chain-related protein, ceruloplasmin |
| | (ferroxidase) |
| 704 | hornerin precursor |
| 712 | complement component 1, s subcomponent, ASPIC, afamin precursor, Vitamin |
| | K-dependent protein |
| 729 | plasma kallikrein precursor, S100 calcium-binding protein A9, Protein S100-A7 |
| | (psoriasin) |
| 744 | gelsolin isoform a precursor |
| 745 | gelsolin isoform a precursor |
| 748 | gelsolin isoform a precursor, coagulation factor XIII, Chain b, Alpha-Ferrous- |
| | Carbonmonoxy |
| 763 | afamin precursor, coagulation factor II precursor, insulin-like growth factor |
| | binding protein, acid labile, histidine-rich glycoprotein precursor, phospholipid |
| | transfer protein isoform a precursor, antithrombin III |
| 764 | glucosamine (N-acetyl)-6-sulfatase precursor, coagulation factor XIII B chain, |
| | gelsolin isoform a precursor |
| 765 | ASPIC, coagulation factor II precursor (unnamed protein), vitronectin (unnamed |
| | protein), histidine-rich glycoprotein precursor, biotinidase precursor |
| 766 | afamin precursor, insulin-like growth factor binding protein, acid labile subunit, |
| | coagulation factor II precursor, alpha-1-B-glycoprotein, thrombin inhibitor, C9 |
| | complement protein |
| 770 | Coagulation factor XIII B chain precursor |
| 776 | insulin-like growth factor binding protein, hemopexin precursor |
| 818 | vanin 1 precursor, biotinidase precursor, vitronectin (unnamed protein), ASPIC |
| 825 | extracellular matrix protein 1 isoform precursor, hemopexin precursor, histidine- |
| | rich glycoprotein, dopamine beta-hydroxylase precursor, peptidoglycan |
| | recognition protein L precursor |
| 827 | Chain A, Crystal Structure of Native Heparin Cofactor Ii, fibrinogen gamma |
| | chain, hemopexin precursor |
| 833 | Extracellular matrix protein1 isoform 1 precursor, inter-alpha (globulin) inhibitor |
| | H1, isoform CRA_b, hemopexin precursor |
| 844 | alpha-1-B-glycoprotein, alpha-2-antiplasmin precursor, complement component 1, |
| | vitronectin precursor (unnamed protein), Vitamin K-dependent protein S |
| | precursor, ASPIC |
| 846 | Vitamin K-dependent protein S precursor, |
| | Chain B, Human Complement Component C3, coagulation factor (unnamed |
| | protein), complement 9, GRP78, afamin precursor |

TABLE 4
Proteins Differentially Expressed Between Healthy vs. LOA (12 sample and 18 sample)

| 2D | | | | |
|---|---|---|---|---|
| Master# | Protein | Mass | pI | Coverage |
| 22 | ceruloplasmin | 116197 | 5.43 | 16% |
| 22 | complement component C3 | 188585 | 6.02 | 5% |
| 22 | Putative | 2269 | 9.79 | 38% |
| 22 | Fibronectin precursor (FN) (Cold-insoluble globulin) (CIG) | 266034 | 5.45 | 4% |
| 22 | Alpha-2-macroglobulin precursor (Alpha-2-M) | 164600 | 6.00 | 3% |
| 22 | Hornerin | 48797 | 9.71 | 3% |
| 22 | complement C4B precursor | 189599 | 7.39 | 3% |
| 22 | mutant beta-actin (beta′-actin).2 | 42128 | 5.22 | 4% |
| 22 | Chain A, Crystallographic Analysis Of The Human Vitamin | 52780 | 5.17 | 10% |
| | D Binding Protein | | | |
| 22 | dermcidin preproprotein | 11391 | 6.08 | 10% |
| 22 | cAMP-specific phosphodiesterase PDE4D5 | 84945 | 5.03 | 1% |
| 22 | hCG22067 | 30319 | 7.98 | 3% |
| 22 | proapolipoprotein | 28944 | 5.45 | 9% |
| 22 | hypothetical protein LOC400867 | 15522 | 10.07 | 4% |
| 22 | Chain I, Crystal Structure Of P13 Alanine Variant Of | 49276 | 6.13 | 8% |
| | Antithrombin | | | |
| 22 | PREDICTED: similar to Cyclin G-associated kinase | 148776 | 9.19 | 4% |
| 29 | chain A . . . uncleaved alpha-1-antitrypsin | 44307 | 5.35 | 55% |
| 29 | cancer-associated SCM-recognition immunedefense- | 46864 | 8.48 | 51% |
| | suppressing and serine protease-protecting peptide, CRISPP | | | |
| | peptide | | | |
| 29 | Putative | 2269 | 9.79 | 38% |
| 76 | hypothetical protein | 272166 | 5.30 | 16% |
| 76 | Chain A, Crystallographic Analysis Of The Human Vitamin | 51183 | 5.17 | 53% |
| | D Binding Protein | | | |
| 76 | Chain B, Structure Of Complement C3b: Insights Into | 103886 | 5.18 | 24% |
| | Complement Activation And Regulation | | | |
| 76 | ceruloplasmin | 115398 | 5.43 | 15% |
| 76 | inter-alpha (globulin) inhibitor H4 (plasma Kallikrein- | 76914 | 5.72 | 9% |
| | sensitive glycoprotein) variant | | | |
| 76 | complement C4B precursor | 188230 | 7.39 | 3% |
| 76 | afamin precursor | 69024 | 5.64 | 20% |
| 76 | Chain A, Antithrombin Iii | 49008 | 5.95 | 25% |
| 76 | trypsin inhibitor | 106647 | 6.58 | 6% |
| 76 | alpha-2-antiplasmin precursor | 54194 | 5.71 | 6% |
| 76 | C9 complement protein | 62974 | 5.49 | 6% |
| 76 | hypothetical protein | 272166 | 5.30 | 16% |
| 76 | Chain A, Crystallographic Analysis Of The Human Vitamin | 51183 | 5.17 | 53% |
| | D Binding Protein | | | |
| 76 | Chain B, Structure Of Complement C3b: Insights Into | 103886 | 5.18 | 24% |
| | Complement Activation And Regulation | | | |
| 76 | ceruloplasmin | 115398 | 5.43 | 15% |
| 76 | inter-alpha (globulin) inhibitor H4 (plasma Kallikrein- | 76914 | 5.72 | 9% |
| | sensitive glycoprotein) variant | | | |
| 76 | complement C4B precursor | 188230 | 7.39 | 3% |
| 76 | afamin precursor | 69024 | 5.64 | 20% |
| 76 | Chain A, Antithrombin Iii | 49008 | 5.95 | 25% |
| 76 | trypsin inhibitor | 106647 | 6.58 | 6% |
| 76 | alpha-2-antiplasmin precursor | 54194 | 5.71 | 6% |
| 76 | C9 complement protein | 62974 | 5.49 | 6% |
| 80 | putative | 2269 | 9.79 | 38% |
| 80 | unnamed protein product | 47777 | 5.60 | 1% |
| 80 | Mitochondrial ribosomal protein L30 | 18678 | 10.10 | 5% |
| 85 | fibronectin 1 isoform 3 preproprotein | 262656 | 5.49 | 26% |
| 85 | ceruloplasmin | 116197 | 5.43 | 26% |
| 85 | alpha-2-macroglobulin precursor | 164600 | 6.00 | 7% |
| 85 | complement component C3 | 188585 | 6.02 | 6% |
| 85 | putative | 2269 | 9.79 | 38% |
| 85 | hemopexin | 13452 | 6.70 | 9% |
| 85 | dermcidin preproprotein | 11391 | 6.08 | 10% |
| 85 | complement component C4A | 194337 | 6.65 | 2% |
| 85 | ectonucleotide pyrophosphatase/phosphodiesterase 5 | 54745 | 5.94 | 2% |
| | (putative function) | | | |
| 85 | PREDICTED: similar to Apolipoprotein(a) precursor | 14926 | 5.79 | 7% |
| | (Apo(a)) (Lp(a)) | | | |
| 85 | factor H | 143710 | 6.28 | 1% |
| 85 | unnamed protein product | 78399 | 8.44 | 1% |
| 85 | cAMP-specific phosphodiesterase PDE4D5 | 84945 | 5.03 | 1% |
| 85 | scavenger receptor class F, member 2 isoform 1 | 96980 | 8.89 | 1% |
| 85 | Chain A, Hr1b Domain From Prk1 | 9054 | 9.77 | 11% |
| 85 | hypothetical protein LOC400867 | 15522 | 10.07 | 4% |
| 96 | hypothetical protein | 272166 | 5.30 | 11% |
| 96 | alpha-2-macroglobulin precursor | 163175 | 6.00 | 8% |
| 96 | complement component C3 | 187046 | 6.02 | 2% |
| 96 | dermcidin preproprotein | 11277 | 6.08 | 16% |
| 96 | hornerin precursor | 282199 | 10.04 | 2% |
| 101 | fibronectin 1 isoform 3 preproprotein | 262656 | 5.49 | 23% |
| 101 | alpha-2-macroglobulin precursor | 164600 | 6.00 | 19% |
| 101 | putative | 2269 | 9.97 | 38% |
| 101 | Plasminogen | 93263 | 6.89 | 1% |
| 101 | cAMP-specific phosphodiesterase PDE4D5 | 84945 | 5.03 | 1% |
| 101 | cardiotrophin-like cytokine factor 1 | 25388 | 8.68 | 3% |
| 102 | fibronectin 1 isoform 3 preproprotein | 262656 | 5.49 | 12% |
| 102 | Alpha-2-macroglobulin precursor (Alpha-2-M) | 164600 | 6.00 | 9% |
| 102 | putative | 2269 | 9.79 | 38% |
| 102 | dermcidin preproprotein | 11391 | 6.08 | 10% |
| 102 | filaggrin 2 | 249296 | 8.45 | 0% |
| 102 | profilaggrin | 11043 | 5.36 | 10% |
| 102 | Chain A, Crystal Structure Of Human Galectin-7 In | 14992 | 7.00 | 8% |
| | Complex With Galactosamine | | | |
| 102 | cAMP-specific phosphodiesterase PDE4D5 | 84945 | 5.03 | 1% |
| 102 | PREDICTED: similar to Cyclin G-associated kinase | 148776 | 9.19 | 1% |
| 103 | hypothetical protein | 252738 | 5.92 | 23% |
| 103 | Alpha-2-macroglobulin precursor (Alpha-2-M) | 164600 | 6.00 | 17% |
| 103 | dermcidin preproprotein | 11391 | 6.08 | 20% |
| 103 | filaggrin 2 | 249296 | 8.45 | 2% |
| 103 | cAMP-specific phosphodiesterase PDE4D5 | 84945 | 5.03 | 1% |
| 103 | Chain A, Hr1b Domain From Prk1 | 9054 | 9.77 | 11% |
| 103 | PREDICTED: similar to ribosomal protein L36 [Pan | 5528 | 10.50 | 21% |
| | troglodytes] | | | |
| 103 | hornerin | 48797 | 9.71 | 6% |
| 103 | PREDICTED: similar to Cyclin G-associated kinase | 148776 | 9.19 | 4% |
| 104 | fibronectin 1 isoform 3 preproprotein | 262656 | 5.49 | 17% |
| 104 | Alpha-2-macroglobulin precursor (Alpha-2-M) | 164600 | 6.00 | 18% |
| 104 | Putative | 2269 | 9.79 | 38% |
| 104 | dermcidin preproprotein | 11391 | 6.08 | 10% |
| 104 | cAMP-specific phosphodiesterase PDE4D5 | 84945 | 5.03 | 1% |
| 111 | no significant hits to report | 0 | 0 | 0 |
| 112 | fibronectin precursor (FN) | 266034 | 5.45 | 25% |
| 112 | complement component C3 | 188585 | 6.02 | 8% |
| 112 | PREDICTED: similar to Apolipoprotein(a) precursor | 14926 | 5.79 | 17% |
| 112 | putative | 2269 | 9.79 | 38% |
| 112 | Shroom-related protein | 218125 | 7.75 | 1% |
| 112 | hypothetical protein LOC400867 | 15522 | 10.07 | 4% |
| 114 | hypothetical protein | 272166 | 5.30 | 25% |
| 114 | Alpha-2-macroglobulin precursor (Alpha-2-M) | 163175 | 6.00 | 13% |
| 114 | complement component C3 | 187046 | 6.02 | 8% |
| 114 | complement component C3b - human (fragments) | 25280 | 4.49 | 23% |
| 114 | lipoprotein, Lp(a) | 130761 | 5.77 | 5% |
| 114 | filaggrin | 434922 | 9.24 | 2% |
| 122 | complement component 3, isoform CRA_a | 143619 | 8.24 | 11% |
| 122 | hypothetical protein | 249203 | 5.43 | 10% |
| 122 | lipoprotein, Lp(a) | 130761 | 5.77 | 4% |
| 122 | hornerin precursor | 282199 | 10.04 | 0% |
| 122 | chain, annexin I | 35018 | 7.77 | 10% |
| 122 | annexin A2 isoform 2 | 38580 | 7.57 | 10% |
| 127 | hypothetical protein | 272166 | 5.30 | 18% |
| 127 | inter-alpha-trypsin inhibitor family heavy chain-related | 103321 | 6.51 | 3% |
| | protein | | | |
| 127 | lipoprotein, Lp(a) | 130761 | 5.77 | 2% |
| 127 | complement component C3 | 187046 | 6.02 | 5% |
| 127 | plasma protease (C1) inhibitor precursor | 55147 | 6.09 | 6% |
| 127 | megakaryocyte stimulating factor | 150998 | 9.53 | 5% |
| 189 | attractin-2 | 146159 | 6.65 | 18% |
| 189 | fibronectin precursor | 260064 | 5.45 | 7% |
| 189 | afamin precursor | 70963 | 5.64 | 10% |
| 189 | antithrombin III | 53041 | 6.32 | 5% |
| 189 | inter-alpha-trypsin inhibitor family heavy | 103549 | 6.51 | 2% |
| 189 | C9 complement protein | 64399 | 5.49 | 5% |
| 189 | thrombin inhibitor | 42901 | 5.33 | 2% |
| 189 | Serpin B8 (cytoplasmic antiproteinase 2) | 43328 | 5.43 | 4% |
| 189 | Frzb precursor | 37243 | 8.75 | 1% |
| 189 | KIAA1481 | 150746 | 6.77 | 0% |
| 194 | Alpha-2-macroglobulin precursor (Alpha-2-M) | 164600 | 6.00 | 34% |
| 194 | putative | 2269 | 9.79 | 38% |
| 194 | complement component C3 | 188585 | 6.02 | 2% |
| 194 | dermcidin preproprotein | 11391 | 6.08 | 10% |
| 194 | factor H | 66430 | 6.28 | 1% |
| 194 | hCG22067 | 30319 | 7.98 | 10% |
| 194 | transient receptor potential cation channel, subfamily V, | 83296 | 6.01 | 1% |
| | member 5 | | | |
| 194 | Human homologue of S. pombe nuc2+ and A. nidulans | 92849 | 6.85 | 0% |
| | bimA | | | |
| 194 | cAMP-specific phosphodiesterase PDE4D5 | 84945 | 5.03 | 1% |
| 215 | alpha 2 macroglobulin | 167505 | 6.06 | 45% |
| 215 | macroglobulin alpha 2 | 162072 | 5.95 | 45% |
| 215 | chain B, complement component C3 | 114238 | 5.55 | 41% |
| 215 | complement factor H | 143654 | 6.23 | 31% |
| 215 | hypothetical protein | 249992 | 5.77 | 4% |
| 215 | inter-alpha-trypsin inhibitor precursor | 101782 | 6.31 | 7% |
| 215 | trypsin inhibitor | 107103 | 6.58 | 7% |
| 215 | hemopexin precursor | 52254 | 6.57 | 13% |
| 215 | regucalcin | 33802 | 5.89 | 5% |
| 215 | annexin A2 | 38808 | 7.57 | 6% |
| 215 | prepro-alpha1 (I) collagen | 139853 | 5.66 | 2% |
| 215 | dipeptidylpeptidase IV | 1599 | 5.91 | 100% |
| 215 | gelsolin isoform a precursor | 86043 | 5.90 | 3% |
| 215 | PREDICTED: similar to Hypothetical acrosin-like protease | 29811 | 8.51 | 9% |
| 215 | branched chain acyltransferase precursor | 53324 | 8.59 | 6% |
| 215 | FLJ00239 protein | 79635 | 7.83 | 1% |
| 215 | aberrant LSLCL | 33536 | 6.82 | 2% |
| 244 | complement factor H isoform a precursor | 138979 | 6.23 | 24% |
| 244 | alpha-2-macroglobulin precursor | 163175 | 6.00 | 13% |
| 244 | chain, x-ray crystal structure of ceruloplasmin | 120009 | 5.41 | 20% |
| 244 | complement C4B precursor | 188230 | 7.39 | 7% |
| 244 | ITIH1 | 52461 | 8.63 | 7% |
| 244 | inter-alpha-trypsin inhibitor family heavy chain-related | 103321 | 6.51 | 6% |
| | protein | | | |
| 244 | hypothetical protein | 246513 | 5.77 | 4% |
| 244 | S100 calcium-binding protein A8 | 10828 | 6.51 | 20% |
| 244 | neutrophil granule peptide HP1 | 3446 | 8.68 | 60% |
| 244 | chain A, crystallographic analysis of Vitamin D binding | 51183 | 5.17 | 15% |
| | protein | | | |
| 244 | dermcidin preproprotein | 11277 | 6.08 | 16% |
| 244 | inter-alpha-trypsin inhibitor heavy chain H2 precursor | 106370 | 6.40 | 10% |
| 244 | Chain A, Structure Of Human Annexin A2 In The Presence | 36460 | 8.32 | 5% |
| | Of Calcium Ions | | | |
| 244 | proapo-A-I protein | 30745 | 5.55 | 4% |
| 244 | hemopexin, isoform CRA_d | 43201 | 6.24 | 6% |
| 244 | complement component C3 | 187046 | 6.02 | 2% |
| 244 | fibrinogen alphaA | 49366 | 5.48 | 5% |
| 247 | complement factor H isoform a precursor | 143654 | 6.23 | 40% |
| 247 | ceruloplasmin | 116197 | 5.43 | 26% |
| 247 | alpha-2-macroglobulin precursor | 164600 | 6.00 | 14% |
| 247 | trypsin inhibitor | 107103 | 6.58 | 16% |
| 247 | fibronectin precursor | 226034 | 5.45 | 10% |
| 247 | inter-alpha (globulin) inhibitor H1 | 101795 | 6.31 | 11% |
| 247 | inter-alpha-trypsin inhibitor family heavy chain-related | 103549 | 6.51 | 8% |
| | protein | | | |
| 247 | chain B, structure of complement C3b | 104912 | 5.18 | 9% |
| 247 | regucalcin | 33802 | 5.89 | 16% |
| 247 | complement C4B precursor | 189599 | 7.39 | 6% |
| 247 | hemopexin precursor | 52254 | 6.57 | 6% |
| 247 | chain A, antithrombin Iii | 49350 | 5.95 | 9% |
| 247 | coactosin-like 1 | 16049 | 5.54 | 11% |
| 247 | cAMP-specific phosphodiesterase PDE4D5 | 84945 | 5.03 | 1% |
| 247 | supported by mouse EST AA538043 (NID: g2284036) | 37423 | 9.28 | 2% |
| 247 | Vitamin D-binding protein precursor (DBP) (Group-specific | 54526 | 5.40 | 3% |
| | component) (Gc-globulin) (VDB) | | | |
| 247 | PREDICTED: hypothetical protein | 23723 | 9.91 | 11% |
| 276 | complement factor H isoform a precursor | 143654 | 6.23 | 45% |
| 276 | inter-alpha-trypsin heavy chain H2 | 106826 | 6.40 | 10% |
| 276 | alpha-2-macroglobulin precursor | 164600 | 6.00 | 7% |
| 276 | inter-alpha-trypsin inhibitor C-terminal | 93745 | 6.02 | 7% |
| 276 | Putative | 2269 | 9.79 | 38% |
| 292 | ceruloplasmin | 116197 | 5.43 | 23% |
| 292 | inter-alpha-trypsin inhibitor family heavy chain-related | 103536 | 6.64 | 11% |
| | protein | | | |
| 292 | putative | 2269 | 9.79 | 38% |
| 292 | Vitamin D-binding protein precursor (DBP) (Group-specific | 54526 | 5.40 | 12% |
| | component) (Gc-globulin) (VDB) | | | |
| 292 | Alpha-2-macroglobulin precursor (Alpha-2-M) | 164600 | 6.00 | 4% |
| 292 | fibronectin precursor | 260064 | 5.45 | 2% |
| 292 | complement C4B precursor | 189599 | 7.39 | 5% |
| 292 | proapolipoprotein | 28944 | 5.45 | 9% |
| 292 | mutant beta-actin (beta′-actin) | 42128 | 5.22 | 2% |
| 292 | PREDICTED: similar to ribosomal protein L36 [Pan | 5528 | 10.50 | 21% |
| | troglodytes] | | | |
| 292 | immunoglobulin lambda light chain VLJ region | 11755 | 8.62 | 9% |
| 292 | annexin A2 isoform 2 | 38808 | 7.57 | 11% |
| 292 | coactosin-like 1 | 16049 | 5.54 | 5% |
| 292 | hypothetical protein LOC400867 | 15522 | 10.07 | 4% |
| 305 | Ceruloplasmin | 116197 | 5.43 | 21% |
| 305 | putative | 2269 | 9.79 | 38% |
| 305 | inter-alpha-trypsin inhibitor family heavy chain-related | 103549 | 6.51 | 5% |
| | protein (IHRP) | | | |
| 305 | Vitamin D-binding protein precursor (DBP) (Group-specific | 54526 | 5.40 | 6% |
| | component) (Gc-globulin) (VDB) | | | |
| 305 | hypothetical protein LOC400867 | 15522 | 10.07 | 4% |
| 306 | ceruloplasmin | 116197 | 5.43 | 18% |
| 306 | inter-alpha-trypsin inhibitor family heavy chain-related | 103549 | 6.51 | 13% |
| | protein (IHRP) | | | |
| 306 | Alpha-2-macroglobulin precursor (Alpha-2-M) | 164600 | 6.00 | 4% |
| 306 | Putative | 2269 | 9.79 | 38% |
| 306 | factor H | 143710 | 6.28 | 2% |
| 306 | fibronectin precursor | 260064 | 5.45 | 1% |
| 306 | inter-alpha-trypsin inhibitor C-terminal | 93745 | 6.02 | 1% |
| 306 | unnamed protein product | 112600 | 8.27 | 0% |
| 306 | complement component C3 | 188585 | 6.02 | 1% |
| 306 | dermcidin preproprotein | 11391 | 6.08 | 10% |
| 306 | coactosin-like 1 | 16049 | 5.54 | 5% |
| 306 | cAMP-specific phosphodiesterase PDE4D5 | 84945 | 5.03 | 1% |
| 327 | complement conponent C3 | 187046 | 6.02 | 20% |
| 327 | alpha-2-macroglobulin precursor | 163175 | 6.00 | 17% |
| 327 | annexin A2 isoform 2 | 38580 | 7.57 | 3% |
| 327 | inter-alpha (globulin) inhibitor H1, isoform CRA_c | 101303 | 6.43 | 2% |
| 327 | hemopexin precursor | 51512 | 6.57 | 7% |
| 327 | ceruloplasmin | 97637 | 5.29 | 3% |
| 327 | putative | 2212 | 9.79 | 38% |
| 333 | inter-alpha-trypsin inhibitor family heavy chain-related | 103321 | 6.51 | 14% |
| | proteins | | | |
| 333 | M130 antigen | 120901 | 5.66 | 10% |
| 333 | ceruloplasmin | 97637 | 5.29 | 15% |
| 333 | afamin precursor | 69024 | 5.64 | 19% |
| 333 | complement C4B | 188230 | 7.39 | 6% |
| 333 | alpha 2 macroglobulin | 166022 | 6.06 | 3% |
| 333 | C9 complement protein | 62974 | 5.49 | 1% |
| 333 | dermcidin preproprotein | 11277 | 6.08 | 10% |
| 333 | collagen, type VI, alpha 1 precursor | 108462 | 5.26 | 6% |
| 333 | trypsin inhibitor | 106647 | 6.58 | 3% |
| 333 | hypothetical protein | 40328 | 8.67 | 9% |
| 333 | thrombin inhibitor | 42559 | 5.33 | 9% |
| 341 | inter-alpha (globulin) inhibitor H4 | 101179 | 6.21 | 20% |
| 341 | ceruloplasmin | 116685 | 5.48 | 18% |
| 341 | afamin precursor | 69024 | 5.64 | 17% |
| 341 | alpha-2-macroglobulin precursor | 163175 | 6.00 | 4% |
| 341 | M130 antigen extracellular variant | 124248 | 5.77 | 4% |
| 341 | Chain, Solution Nmr Structure Of Recombinant Human | 10982 | 5.38 | 12% |
| | Cystatin A Under The Condition Of Ph 3.8 And 310k | | | |
| 341 | hypothetical protein | 40328 | 8.67 | 7% |
| 345 | ceruloplasmin | 115398 | 5.43 | 15% |
| 345 | inter-alpha-trypsin inhibitor family heavy chain-related | 103321 | 6.51 | 11% |
| | protein | | | |
| 345 | glycosylphosphatidylinositol specific phospholipase D1 | 93272 | 5.96 | 5% |
| 345 | M130 antigen extracellular variant | 124248 | 5.77 | 8% |
| 345 | chain B, structure of complement C3b | 103886 | 5.18 | 10% |
| 345 | alpha-2-macroglobulin precursor | 163175 | 6.00 | 7% |
| 345 | hypothetical protein | 240492 | 5.15 | 2% |
| 345 | Aggrecan core protein precursor (Cartilage-specific | 250040 | 4.10 | 1% |
| | proteoglycan core protein) | | | |
| 345 | Chain A, Structure Of Human Annexin A2 In The Presence | 36460 | 8.32 | 5% |
| | Of Calcium Ions | | | |
| 348 | ceruloplasmin | 115398 | 5.43 | 16% |
| 348 | unknown | 61937 | 6.07 | 19% |
| 348 | alpha-2-macroglobulin precursor | 163175 | 6.00 | 7% |
| 348 | inter-alpha (globulin) inhibitor H4 | 101179 | 6.21 | 6% |
| 348 | serum paraoxonase/arylesterase 1 | 39724 | 5.08 | 11% |
| 353 | chain b, complement component C3 | 114238 | 5.55 | 31% |
| 353 | ceruloplasmin | 116197 | 5.43 | 20% |
| 353 | phosphatidylinositol-glycan-specific phospholipase D1 | 92943 | 5.91 | 16% |
| | precursor | | | |
| 353 | alpha-2-macroglobulin precursor | 164600 | 6.00 | 7% |
| 353 | inter-alpha-trypsin inhibitor family heavy chain-related | 103549 | 6.51 | 3% |
| | protein | | | |
| 353 | regucalcin | 33802 | 5.89 | 5% |
| 353 | complement factor H isoform a precursor | 143654 | 6.23 | 1% |
| 353 | ASPIC | 71448 | 4.98 | 6% |
| 353 | fibronectin precursor | 260064 | 5.45 | 3% |
| 353 | alpha-1-antichymotrypsin | 48834 | 5.79 | 6% |
| 353 | creatine kinase M (EC 2.7.3.2) | 8024 | 9.90 | 25% |
| 353 | Chain A, Human Serum Albumin Mutant R218p | 68366 | 5.62 | 3% |
| | Complexed With Thyroxine (3,3′,5,5′-Tetraiodo-L- | | | |
| | Thyronine) | | | |
| 353 | Aggrecan core protein precursor (Cartilage-specific | 251979 | 4.10 | 0% |
| | proteoglycan core protein) 1 | | | |
| 353 | C1-inhibitor | 32745 | 8.85 | 2% |
| 353 | dermcidin preproprotein | 11391 | 6.08 | 10% |
| 353 | alpha2-HS glycoprotein | 36268 | 5.20 | 9% |
| 353 | cAMP-specific phosphodiesterase PDE4D5 | 84945 | 5.03 | 1% |
| 357 | ceruloplasmin | 115398 | 5.43 | 36% |
| 357 | Chain B, Structure Of Complement C3b: Insights Into | 103886 | 5.18 | 18% |
| | Complement Activation And Regulation | | | |
| 357 | inter-alpha (globulin) inhibitor H4 (plasma Kallikrein- | 101179 | 6.21 | 20% |
| | sensitive glycoprotein), isoform CRA_b | | | |
| 357 | serum vitamin D-binding protein precursor | 53015 | 5.40 | 35% |
| 357 | Chain A, Antithrombin Iii | 49008 | 5.95 | 31% |
| 357 | M130 antigen | 120901 | 5.66 | 12% |
| 357 | afamin precursor | 69024 | 5.64 | 15% |
| 357 | Alpha 2 macroglobulin variant | 164030 | 6.00 | 9% |
| 357 | complement component C4A | 192741 | 6.65 | 3% |
| 357 | annexin A2 isoform 2 | 38580 | 7.57 | 10% |
| 357 | trypsin inhibitor | 106647 | 6.58 | 5% |
| 357 | Serpin B8 (Cytoplasmic antiproteinase 2) (CAP-2) (CAP2) | 42758 | 5.43 | 8% |
| | (Protease inhibitor 8) | | | |
| 357 | C9 complement protein | 62974 | 5.49 | 2% |
| 357 | COL6A1 protein | 48594 | 5.60 | 8% |
| 357 | C-reactive protein | 25091 | 6.32 | 3% |
| 357 | alpha-2-antiplasmin precursor | 54194 | 5.71 | 4% |
| 357 | alpha-1 (III) collagen | 96442 | 9.28 | 3% |
| 357 | thrombin inhibitor | 42559 | 5.33 | 12% |
| 357 | immunoglobulin heavy chain variable region | 12733 | 7.92 | 27% |
| 361 | Ceruloplasmin | 115398 | 5.53 | 38% |
| 361 | Chain B, Structure of Complement C3b | 103886 | 5.18 | 28% |
| 361 | ChainA, Antithrombin Iii | 49008 | 5.95 | 23% |
| 361 | inter-alpha-trypsin inhibitor family heavy chain-related | 103321 | 6.51 | 15% |
| | protein | | | |
| 361 | serum vitamin D-binding protein precursor | 53015 | 5.40 | 32% |
| 361 | alpha 2 macroglobulin | 166022 | 6.06 | 8% |
| 361 | M130 antigen cytoplasmic variant 2 | 125271 | 5.68 | 6% |
| 361 | serpin peptidase inhibitor, clade I | 46116 | 5.08 | 7% |
| 361 | complement C4B precursor | 188230 | 7.39 | 1% |
| 361 | PRSS3 | 26470 | 6.86 | 8% |
| 361 | collagen type IV | 4456 | 12.54 | 30% |
| 361 | cAMP-specific phosphodiesterase | 84375 | 5.03 | 1% |
| 361 | alpha-1-B-glycoprotein | 51908 | 5.65 | 3% |
| 361 | Ceruloplasmin | 115398 | 5.43 | 38% |
| 361 | Chain B, Structure of Complement C3b | 103886 | 5.18 | 28% |
| 361 | ChainA, Antithrombin Iii | 49008 | 5.95 | 23% |
| 361 | serum vitamin D-binding precursor | 53015 | 5.40 | 32% |
| 361 | inter-alpha-trypsin inhibitor family heavy chain-related | 103321 | 6.51 | 15% |
| | protein (IHRP) | | | |
| 361 | alpha 2 macroglobulin | 166022 | 6.06 | 8% |
| 361 | M130 antigen cytoplasmic variant 2 | 125271 | 5.68 | 6% |
| 361 | serpin peptidase inhibitor, clade I (pancpin), member 2 | 46116 | 5.08 | 7% |
| 361 | complement C4B precursor | 188230 | 7.39 | 1% |
| 361 | PRSS3 protein | 26470 | 6.86 | 8% |
| 361 | collagen type IV: alpha1 chain | 4456 | 12.54 | 30% |
| 361 | cAMP-specific phosphodiesterase PDE4D5 | 84375 | 5.03 | 1% |
| 361 | alpha-1-B-glycoprotein - human | 51908 | 5.65 | 3% |
| 362 | ceruloplasmin | 115398 | 5.43 | 33% |
| 362 | Chain A, Antithrombin | 49008 | 5.95 | 28% |
| 362 | Chain B, Structure of Complement C3b | 103886 | 5.18 | 19% |
| 362 | inter-alpha-trypsin family heavy chain-related protein | 103321 | 6.51 | 14% |
| 362 | serum vitamin D-binding protein precursor | 53015 | 5.40 | 34% |
| 362 | alpha-2-macroglobulin precursor | 163175 | 6.00 | 5% |
| 362 | prepro-C3b/C4B inactivator | 65725 | 7.72 | 3% |
| 362 | glutathione transferase M3 | 26671 | 5.37 | 8% |
| 362 | complement C4B | 188230 | 7.39 | 3% |
| 362 | trypsin inhibitor | 106647 | 6.58 | 1% |
| 362 | PREDICTED: similar to Hornerin | 187937 | 9.82 | 1% |
| 362 | Desmoplakin | 201237 | 6.68 | 2% |
| 362 | M130 antigen | 120901 | 5.66 | 5% |
| 362 | unnamed protein product | 40903 | 9.50 | 7% |
| 362 | small proline-rich protein 2G | 8152 | 8.30 | 43% |
| 363 | ceruloplasmin | 115398 | 5.43 | 26% |
| 363 | inter-alpha-trypsin inhibitor family heavy chain-related | 103308 | 6.64 | 6% |
| | protein | | | |
| 363 | dermcidin preproprotein | 11277 | 6.08 | 10% |
| 363 | small proline-rich protein 2D | 7900 | 8.77 | 30% |
| 363 | filaggrin 2 | 247928 | 8.45 | 0% |
| 363 | desmoplakin I | 331571 | 6.44 | 1% |
| 363 | nuclear transcription factor, X-box binding-like 1, isoform | 69844 | 8.88 | 1% |
| | CRA_e | | | |
| 370 | ceruloplasmin | 116197 | 5.43 | 20% |
| 370 | alpha-2-macroglobulin precursor | 164600 | 6.00 | 14% |
| 370 | inter-alpha-trypsin inhibitor | 103549 | 6.51 | 8% |
| 370 | complement C4B preccursor | 189599 | 7.39 | 3% |
| 370 | complement component C3 | 188585 | 6.02 | 3% |
| 370 | factor H | 143710 | 6.28 | 1% |
| 370 | fibrinogen gamma chain | 50077 | 5.61 | 8% |
| 370 | annexin A2 | 38808 | 7.57 | 3% |
| 374 | alpha 2 macroglobulin | 166022 | 6.06 | 17% |
| 374 | ceruloplasmin | 115398 | 5.43 | 17% |
| 374 | hCG40889, isoform CRA_a | 132000 | 5.98 | 10% |
| 374 | inter-alpha-trypsin inhibitor family heavy chain-related | 103321 | 6.51 | 9% |
| | protein (IHRP) | | | |
| 374 | inter-alpha-trypsin inhibitor C-terminal | 93402 | 6.02 | 5% |
| 374 | Chain B, Human Complement Component C3 | 112869 | 5.55 | 4% |
| 374 | hemopexin precursor | 51512 | 6.57 | 8% |
| 378 | alpha 2 macroglobulin | 166022 | 6.06 | 17% |
| 378 | ceruloplasmin | 115398 | 5.43 | 16% |
| 378 | chain, lactoferrin | 76076 | 8.40 | 18% |
| 378 | hypothetical protein | 70808 | 5.86 | 10% |
| 378 | polymeric immunoglobulin receptor | 83232 | 5.58 | 12% |
| 378 | protein Rei, Bence-Jones | 23494 | 8.75 | 12% |
| 378 | chain, contribution of hydrogen bonds to lysozyme | 14675 | 9.30 | 41% |
| 378 | deleted in malignant brain tumors 1 isoform b precursor | 59167 | 4.69 | 10% |
| | variant | | | |
| 378 | chain A, crystal structure of human neutrophil peptide 2 | 3429 | 8.67 | 86% |
| 378 | unnamed protein product | 64729 | 8.40 | 12% |
| 378 | Ig A1 Bur | 73331 | 9.24 | 9% |
| 378 | PREDICTED: similar to Mucin-5B precursor | 540248 | 6.12 | 2% |
| 378 | Annexin A2 | 38552 | 7.57 | 9% |
| 378 | Chain B, Structure Of Complement C3b: Insights Into | 103886 | 5.18 | 6% |
| | Complement Activation And Regulation | | | |
| 378 | C20orf114 | 52434 | 6.67 | 9% |
| 378 | factor H | 139034 | 6.28 | 4% |
| 378 | unnamed protein product | 10931 | 9.19 | 19% |
| 378 | unnamed protein product | 66412 | 5.62 | 9% |
| 378 | KRT73 protein | 41985 | 8.42 | 7% |
| 378 | neutrophil elastase precursor | 20748 | 9.30 | 13% |
| 378 | mutant beta-actin (beta′-actin) | 41786 | 5.22 | 5% |
| 384 | hypothetical protein | 164632 | 6.00 | 35% |
| 384 | ceruloplasmin | 116197 | 5.43 | 11% |
| 384 | inter-alpha-trypsin inhibitor family heavy chain-related | 103549 | 6.51 | 15% |
| | protein | | | |
| 384 | factor H | 143710 | 6.28 | 8% |
| 384 | chain b, structure of comlement C3b | 104912 | 5.18 | 9% |
| 384 | inter-alpha-trypsin inhibitor C-terminal | 93745 | 6.02 | 2% |
| 384 | annexin A2 isoform 2 | 38808 | 7.57 | 6% |
| 384 | hemopexin precursor | 52254 | 6.57 | 8% |
| 384 | regucalcin | 33802 | 5.89 | 9% |
| 384 | fibrinogen gamma chain | 50077 | 5.61 | 2% |
| 384 | cardiotrophin-like cytokine factor 1 | 25388 | 8.68 | 3% |
| 384 | cAMP-specific phosphodiesterase PDE4D5 | 84945 | 5.03 | 1% |
| 389 | alpha-2-macroglobulin precursor | 164600 | 6.00 | 22% |
| 389 | Chain B, Complement Component C3 | 114238 | 5.55 | 15% |
| 389 | annexin A2 isoform 2 | 38808 | 7.57 | 6% |
| 389 | gelsolin isoform a precursor | 86043 | 5.90 | 5% |
| 389 | complement component C6 precursor | 108367 | 6.39 | 2% |
| 389 | inter-alpha-trypsin inhibitor C-terminal | 93745 | 6.02 | 2% |
| 389 | fibronectin precursor | 260064 | 5.45 | 1% |
| 389 | cAMP-specific phosphodiesterase PDE4D5 | 84945 | 5.03 | 1% |
| 389 | 60/63 kda phosphoglucomutase | 2279 | 4.03 | 47% |
| 389 | beta-actin | 7908 | 6.75 | 29% |
| 389 | hCG22067 | 30319 | 7.98 | 3% |
| 393 | alpha-2-macroglobulin precursor | 164600 | 6.00 | 25% |
| 393 | Chain B, Human Complement Conponent C3 | 114238 | 5.55 | 28% |
| 393 | inter-alpha (globulin) inhibitor H4 | 101521 | 6.21 | 3% |
| 393 | annexin A2 isoform 2 | 38808 | 7.57 | 2% |
| 393 | sex-determination protein homolog Fem1a | 22632 | 7.22 | 10% |
| 399 | alpha-2-macroglobulin precursor | 164600 | 6.00 | 22% |
| 399 | complement component C3 | 188585 | 6.02 | 7% |
| 399 | complement component C6 precursor | 108367 | 6.39 | 3% |
| 399 | annexin A2 isoform 2 | 38808 | 7.57 | 6% |
| 399 | inter-alpha-trypsin inhibitor | 93745 | 6.02 | 1% |
| 399 | dermcidin preproprotein | 11391 | 6.08 | 10% |
| 399 | Chain A, Crystal Structure Analysis Of The Bb Segment Of | 56816 | 7.16 | 9% |
| | Factor B | | | |
| 399 | gelsolin isoform a precursor | 86043 | 5.90 | 3% |
| 399 | fibronectin precursor | 260064 | 5.45 | 1% |
| 399 | cAMP-specific phosphodiesterase PDE4D5 | 84945 | 5.03 | 1% |
| 399 | Bruton's agammaglobulinemia tyrosine kinase | 76625 | 8.05 | 4% |
| 413 | ceruloplasmin | 116197 | 5.43 | 28% |
| 413 | inter-alpha-trypsin inhibitor family havey chain-related | 103536 | 6.64 | 26% |
| | protein | | | |
| 413 | alpha-2-macroglobulin precursor | 164600 | 6.00 | 21% |
| 413 | chain b, structure of complement C3b | 104912 | 5.18 | 16% |
| 413 | IgG Fc binding protein | 81017 | 5.56 | 5% |
| 413 | pregnancy-zone protein | 165215 | 5.97 | 7% |
| 413 | complement factor H isoform a precursor | 143654 | 6.23 | 5% |
| 413 | complement component C4A | 194337 | 6.65 | 1% |
| 413 | regucalcin | 33802 | 5.98 | 5% |
| 413 | hemopexin precursor | 52254 | 6.57 | 6% |
| 413 | fibronectin precursor | 260064 | 5.45 | 0% |
| 413 | fibrinogen gamma chain | 50077 | 5.61 | 10% |
| 413 | cAMP-specific phosphodiesterase PDE4D5 | 84945 | 5.03 | 1% |
| 415 | alpha-2 macroglobulin precursor | 163175 | 6.00 | 14% |
| 415 | complement component C6 precursor peptide | 104718 | 6.39 | 22% |
| 415 | gelsolin isoform a precursor | 85644 | 5.90 | 16% |
| 415 | ceruloplasmin | 115398 | 5.43 | 6% |
| 415 | vinculin isoform VCL | 116649 | 5.83 | 9% |
| 415 | complement component C3 | 187046 | 6.02 | 1% |
| 415 | annexin A2 isoform 2 | 38580 | 7.57 | 11% |
| 418 | complement component C6 precursor | 108367 | 6.39 | 23% |
| 418 | vinculin isoform VCL | 117220 | 5.83 | 24% |
| 418 | alpha-2-macroglobulin precursor | 164600 | 6.00 | 17% |
| 418 | complement factor B | 86819 | 6.55 | 15% |
| 418 | complement factor H isoform a precursor | 143654 | 6.23 | 1% |
| 418 | complement component C3 | 188585 | 6.02 | 2% |
| 418 | complement protein C7 precursor | 96647 | 6.09 | 7% |
| 418 | annexin A2 isoform 2 | 38808 | 7.57 | 2% |
| 418 | trypsin inhibitor | 107103 | 6.58 | 1% |
| 418 | gelsolin isoform a precursor | 86043 | 5.90 | 3% |
| 418 | chondrosarcoma-associated protein 2 | 65660 | 6.26 | 1% |
| 418 | Chain A, Hr1b Domain From Prk1 | 9054 | 9.77 | 11% |
| 418 | epidermal growth factor receptor | 90700 | 8.39 | 1% |
| 421 | complement component C6 precurosr | 108367 | 6.39 | 26% |
| 421 | alpha 2 macroglobulin | 167505 | 6.06 | 20% |
| 421 | complement factor B | 86819 | 6.55 | 19% |
| 421 | vinculin isoform VCL | 117220 | 5.83 | 15% |
| 421 | annexin A2 isoform 2 | 38808 | 7.57 | 9% |
| 421 | complement component C3 | 188585 | 6.02 | 0% |
| 421 | Plasminogen | 93263 | 6.89 | 1% |
| 421 | cAMP-specific phosphodiesterase PDE4D5 | 84945 | 5.03 | 1% |
| 429 | inter-alpha (globulin) inhibitor H4 | 101179 | 6.21 | 19% |
| 429 | phosphatidylinositol-glycan-specific phospholipase D1 | 92316 | 5.91 | 13% |
| | precursor | | | |
| 429 | ceruloplasmin | 97637 | 5.29 | 8% |
| 429 | alpha 2 macroglobulin | 166022 | 6.06 | 7% |
| 429 | chain B, structure of complement C3b | 103886 | 5.18 | 6% |
| 429 | serpin peptidase inhibitor, clade A | 37044 | 6.00 | 12% |
| 429 | dermcidin preproprotein | 11277 | 6.08 | 13% |
| 429 | PREDICTED: similar to filaggrin 2 | 186543 | 7.67 | 0% |
| 429 | complement component C6 precursor peptide | 104718 | 6.39 | 4% |
| 429 | factor H | 139034 | 6.28 | 5% |
| 429 | unnamed protein | 69241 | 5.53 | 4% |
| 429 | hypothetical protein | 40325 | 8.67 | 14% |
| 429 | trypsin inhibitor | 106647 | 6.58 | 4% |
| 429 | inter-alpha (globulin) inhibitor H1, isoform CRA_b | 99357 | 6.59 | 2% |
| 429 | Ig heavy chain V region - human | 1981 | 9.08 | 5% |
| 435 | inter-alpha (globulin) inhibitor H4 | 101521 | 6.21 | 25% |
| 435 | chain B, structure of complement C2b | 104912 | 5.18 | 13% |
| 435 | ceruloplasmin | 116197 | 5.43 | 8% |
| 435 | unnamed protein product | 70723 | 5.53 | 8% |
| 435 | phosphatidylinositol-glycan-specific phospholipase D1 | 92943 | 5.91 | 7% |
| | precursor | | | |
| 435 | complement component C4A | 194337 | 6.65 | 1% |
| 435 | alpha 2 macroglobulin | 167505 | 6.06 | 5% |
| 435 | factor H | 143710 | 6.28 | 2% |
| 435 | afamin precursor | 70963 | 5.64 | 6% |
| 435 | annexin A2 isoform 2 | 38808 | 7.57 | 6% |
| 435 | fibronectin precursor | 260064 | 5.45 | 0% |
| 435 | trypsin inhibitor | 107103 | 6.58 | 1% |
| 435 | dermcidin preproprotein | 11391 | 6.08 | 10% |
| 435 | complement component C6 precursor peptide | 108367 | 6.39 | 1% |
| 435 | lumican | 38717 | 6.16 | 8% |
| 435 | Cartilage oligomeric matrix protein precursor (COMP) | 85403 | 4.34 | 4% |
| 435 | Plasminogen | 93263 | 6.89 | 1% |
| 438 | inter-alpha (globulen) inhibitor H4 | 101521 | 6.21 | 22% |
| 438 | Ceruloplasmin | 180249 | 5.29 | 8% |
| 438 | complement component C3 | 188585 | 6.02 | 0% |
| 438 | glycosylphosphatidylinositol phospholipase D | 92931 | 5.91 | 4% |
| 438 | alpha-albumin | 4647 | 4.10 | 35% |
| 445 | none | | | |
| 447 | LOST | | | |
| 457 | inter-alpha (globulin) inhibitor H4 | 101521 | 6.21 | 26% |
| 457 | ceruloplasmin | 116197 | 5.43 | 20% |
| 457 | Chain B, Structure of Complement C3b | 104912 | 5.18 | 22% |
| 457 | Vitamin D-binding protein precursor | 54526 | 5.40 | 15% |
| 457 | afamin precursor | 7093 | 5.64 | 14% |
| 457 | serine proteinase inhibitor | 43004 | 5.61 | 7% |
| 457 | complement component C4B | 40795 | 5.19 | 12% |
| 457 | annexin A2 isoform 2 | 38808 | 7.57 | 6% |
| 457 | thrombin inhibitor | 42901 | 5.33 | 3% |
| 457 | alpha-1-B-glycoprotein - human | 52479 | 5.65 | 2% |
| 457 | thrombospondin-4 | 108415 | 4.44 | 3% |
| 457 | C9 complement protein | 64399 | 5.49 | 1% |
| 457 | immunoglobulin G kappa chain | 24212 | 6.18 | 6% |
| 457 | antithrombin III | 53041 | 6.32 | 3% |
| 457 | lamin-like protein | 16417 | 10.33 | 5% |
| 466 | complement component C3 | 188585 | 6.02 | 25% |
| 466 | alpha-2-macroglobulin precursor | 164600 | 6.00 | 3% |
| 470 | complement component C3 | 188585 | 6.02 | 26% |
| 470 | alpha-2-macroglobulin precursor | 164600 | 6.00 | 7% |
| 470 | mitochondrial ribosomal protein | 18678 | 10.01 | 5% |
| 472 | inter-alpha (globulin) inhibitor H4 | 101521 | 6.21 | 22% |
| 472 | ceruloplasmin | 116197 | 5.43 | 22% |
| 472 | Vitamin-D-binding protein precursor | 54526 | 5.40 | 26% |
| 472 | Chain B, Structure of Complement C3b | 104912 | 5.18 | 17% |
| 472 | afamin precursor | 70963 | 5.64 | 9% |
| 472 | dermcidin prepropprotein | 11391 | 6.08 | 10% |
| 472 | glutathione transferase M3 | 27127 | 5.37 | 4% |
| 472 | small proline-rich protein 2D | 8584 | 8.77 | 30% |
| 472 | FAM124A | | 5.47 | 1% |
| 472 | antithrombin | 14064 | 5.91 | 24% |
| 472 | plasminogen | 93263 | 6.89 | 1% |
| 472 | complement C4B precursor | 189599 | 7.39 | 1% |
| 472 | hCG22067 | 30319 | 7.98 | 6% |
| 473 | ceruloplasmin | 116197 | 5.43 | 17% |
| 473 | inter-alpha-trypsin inhibitor | 103549 | 6.51 | 13% |
| 473 | complement component C3 | 188585 | 6.02 | 0% |
| 475 | plasminogen | 90526 | 6.89 | 25% |
| 475 | complement component 3 precursor | 187030 | 6.02 | 9% |
| 475 | complement C5 precursor | 188212 | 6.11 | 10% |
| 475 | poly-Ig receptor | 75474 | 5.38 | 2% |
| 475 | plasminogen 1-69 | 7818 | 4.72 | 30% |
| 475 | annexin A2 isoform 2 | 38580 | 7.57 | 3% |
| 475 | TPA: Hornerin | 282204 | 10.04 | 2% |
| 476 | ceruloplasmin | 116197 | 5.43 | 13% |
| 476 | inter-alpha-trypsin inhibitor family heavy chain | 103549 | 6.51 | 12% |
| 476 | lymphoid-rectricted membrane protein | 56691 | 5.44 | 3% |
| 477 | complement factor B | 86819 | 6.55 | 19% |
| 477 | putative | 2269 | 9.79 | 38% |
| 477 | dermcidin preproprotein | 11391 | 6.08 | 20% |
| 477 | complement protein C7 precursor | 96647 | 6.09 | 4% |
| 477 | plasminogen | 93233 | 7.04 | 2% |
| 477 | Alpha-2-macroglobulin precursor (Alpha-2-M) | 164600 | 6.00 | 1% |
| 477 | hCG22067 | 30319 | 7.98 | 3% |
| 477 | unnamed protein product | 84549 | 7.23 | 1% |
| 477 | filaggrin 2 | 249296 | 8.45 | 0% |
| 477 | PREDICTED: similar to Cyclin G-associated kinase | 148776 | 7.67 | 0% |
| 477 | hCG2044987 | 11472 | 9.73 | 8% |
| 481 | ceruloplasmin | 116197 | 5.43 | 27% |
| 481 | Inter-alpha-trypsin inhibitor heavy chain H4 precursor (ITI | 103489 | 6.51 | 15% |
| | heavy chain H4) (Inter-alpha-inhibitor heavy chain 4) | | | |
| 481 | putative | 2269 | 9.79 | 38% |
| 481 | complement component C3 | 188585 | 6.02 | 3% |
| 481 | Alpha-2-macroglobulin precursor (Alpha-2-M) | 164600 | 6.00 | 2% |
| 481 | dermcidin preproprotein | 11391 | 6.08 | 10% |
| 481 | Cartilage oligomeric matrix protein precursor (COMP) | 85403 | 4.34 | 1% |
| 481 | coactosin-like 1 | 16049 | 5.54 | 16% |
| 481 | cAMP-specific phosphodiesterase PDE4D5 | 84945 | 5.03 | 1% |
| 482 | ceruloplasmin | 116197 | 5.43 | 17% |
| 482 | mutant beta-actin | 42128 | 5.22 | 22% |
| 482 | chain b, structure of complement C3b | 104912 | 5.18 | 15% |
| 482 | inter-alpha-trypsin inhibitor family havey chain-related | 103549 | 6.51 | 13% |
| | protein | | | |
| 482 | alpha-2-macroglobulin precursor | 164600 | 6.00 | 5% |
| 482 | regucalcin | 33802 | 5.89 | 9% |
| 482 | complement C4B precursor | 189599 | 7.39 | 2% |
| 482 | ATP synthase . . . | 59828 | 9.16 | 3% |
| 482 | Ca ATPase SERCA1 | 110654 | 5.06 | 4% |
| 482 | dermcidin preproprotein | 11391 | 6.08 | 10% |
| 482 | fibrinogen gamma chain | 50077 | 5.61 | 4% |
| 482 | annexin A2, isoform CRA_c | 32600 | 5.93 | 9% |
| 482 | fibronectin precursor | 260064 | 5.45 | 0% |
| 482 | inter-alpha-trypsin inhibitor C-terminal | 93745 | 6.02 | 2% |
| 482 | cartilage oligomeric matrix protein precursor | 85403 | 4.34 | 2% |
| 482 | Human basement membrane heparan sulfate proteoglycan | 479812 | 6.10 | 0% |
| | core protein | | | |
| 482 | hemopexin precursor | 52254 | 6.57 | 4% |
| 482 | small proline-rich protein | 8676 | 9.07 | 25% |
| 482 | unnamed protein product | 71246 | 5.92 | 1% |
| 482 | hypothetical protein LOC60686 | 59375 | 5.71 | 1% |
| 482 | cardiotrophin-like cytokine factor 1 | 25388 | 8.86 | 3% |
| 482 | unnamed protein product | 57976 | 5.80 | 3% |
| 482 | cytochrome C oxidase II subunit | 21095 | 4.68 | 3% |
| 482 | cAMP-specific phosphodiesterase PDE4D5 | 84945 | 5.03 | 1% |
| 484 | ceruloplasmin | 97637 | 5.29 | 14% |
| 484 | hypothetical protein | 70808 | 5.86 | 12% |
| 484 | complement component C3 | 187046 | 6.02 | 6% |
| 484 | alpha-2-macroglobulin precursor | 163175 | 6.00 | 1% |
| 484 | Cartilage oligomeric matrix protein precursor (COMP) | 82780 | 4.34 | 4% |
| 484 | inter-alpha (globulin) inhibitor H2, isoform CRA_a | 106502 | 6.48 | 4% |
| 484 | complement component C4A | 193541 | 6.79 | 1% |
| 484 | hypothetical protein | 122035 | 6.20 | 2% |
| 484 | peptide, salivary low MW | 1068 | 8.75 | 100% |
| 484 | dermcidin preproprotein | 11277 | 6.08 | 19% |
| 555 | alpha-2-macroglobulin precursor | 164600 | 6.00 | 15% |
| 555 | fibrinogen gamma | 46823 | 5.54 | 24% |
| 555 | inter-alpha-trypsin inhibitor | 93745 | 6.02 | 2% |
| 555 | putative | 2269 | 9.97 | 38% |
| 555 | complement C1r subcomponent precursor | 81661 | 5.89 | 2% |
| 555 | complement component C3 | 188585 | 6.02 | 2% |
| 555 | talin | 271828 | 5.75 | 1% |
| 555 | annexin A2 isoform 2 | 38808 | 7.57 | 6% |
| 555 | hemopexin precursor | 52254 | 6.57 | 9% |
| 555 | plasminogen | 93233 | 7.04 | 4% |
| 555 | dermcidin preproprotein | 11391 | 6.08 | 10% |
| 555 | complement factor B | 86819 | 6.55 | 2% |
| 555 | ubiquitin | 8446 | 6.56 | 12% |
| 555 | gelsolin isoform a precursor | 86043 | 5.90 | 1% |
| 555 | cAMP-specific phosphodiesterase PDE4D5 | 84945 | 5.03 | 1% |
| 555 | unnamed protein product | 112600 | 8.27 | 0% |
| 583 | lumican | 38717 | 6.16 | 34% |
| 583 | C1-inhibitor | 32745 | 8.85 | 21% |
| 583 | putative | 2269 | 9.79 | 38% |
| 583 | filaggrin 2 | 249296 | 8.45 | 0% |
| 583 | Chain A, High Resolution Solution Nmr Structure Of | 11629 | 4.82 | 26% |
| | Mixed Disulfide Intermediate Between Mutant Human | | | |
| | Thioredoxin And A 13 Residue Peptide Comprising Its | | | |
| | Target Site In Human Nfkb | | | |
| 583 | dermcidin preproprotein | 11391 | 6.08 | 10% |
| 583 | inter-alpha-trypsin inhibitor C-terminal | 93745 | 6.02 | 2% |
| 583 | caspase 14 precursor | 27947 | 5.44 | 12% |
| 583 | SCP-1 | 114424 | 5.84 | 2% |
| 583 | unnamed protein product | 14405 | 8.03 | 9% |
| 583 | creatine kinase M (EC 2.7.3.2) | 8024 | 9.90 | 25% |
| 583 | cAMP-specific phosphodiesterase PDE4D5 | 84945 | 5.03 | 1% |
| 584 | alpha-2-macroglobulin precursor | 164600 | 6.00 | 11% |
| 584 | complement component 7 precursor | 96650 | 6.09 | 16% |
| 584 | complement factor B | 86819 | 6.55 | 14% |
| 584 | putative | 2269 | 9.79 | 38% |
| 584 | unnamed protein product | 84549 | 7.23 | 0% |
| 584 | Chain A, Hr1b Domain From Prk1 | 9054 | 9.77 | 11% |
| 586 | complement component 7 precursor | 93457 | 6.09 | 27% |
| 586 | complement factor B | 85450 | 6.55 | 26% |
| 586 | Chain A, antithrombin Iii | 49008 | 5.95 | 42% |
| 586 | unnamed protein product | 83180 | 7.23 | 8% |
| 586 | alpha-2-macroglobulin precursor | 163175 | 6.00 | 8% |
| 586 | serpin peptidase inhibitor, clade F | 57016 | 6.47 | 13% |
| 586 | PREDICTED: hypothetical protein | 31731 | 11.91 | 6% |
| 588 | complement factor B | 86819 | 6.55 | 35% |
| 588 | complement component 7 precursor | 96650 | 6.09 | 22% |
| 588 | alpha-2-macroglobulin precursor | 164600 | 6.00 | 7% |
| 588 | unnamed protein product | 84549 | 7.23 | 5% |
| 588 | putative | 2269 | 9.79 | 38% |
| 588 | unnamed protein product | 112600 | 8.27 | 0% |
| 588 | hCG2041887 | 9274 | 9.80 | 11% |
| 588 | centrin | 19552 | 4.62 | 7% |
| 589 | plasma protease (C1) inhibitor precursor | 55375 | 6.09 | 15% |
| 589 | putative | 2269 | 9.79 | 38% |
| 589 | trypsin inhibitor | 107103 | 6.58 | 8% |
| 589 | lumican | 38717 | 6.16 | 25% |
| 589 | complement component 1, s subcomponent | 78174 | 4.86 | 8% |
| 589 | Biotinidase precursor | 59760 | 5.50 | 6% |
| 589 | Inter-alpha-trypsin inhibitor heavy chain H3 precursor (ITI | 99401 | 5.61 | 4% |
| | heavy chain H3) (Inter-alpha-inhibitor heavy chain 3) | | | |
| | (Serum-derived hyaluronan-associated protein) (SHAP) | | | |
| 589 | Vitamin K-dependent protein S precursor | 77127 | 5.48 | 7% |
| 589 | dermcidin preproprotein | 11391 | 6.08 | 10% |
| 589 | coagulation factor XII | 68618 | 7.94 | 2% |
| 589 | hCG22067 | 30319 | 7.98 | 3% |
| 589 | cadherin-5 | 87288 | 5.19 | 1% |
| 589 | alpha-1-antichymotrypsin | 48834 | 5.79 | 9% |
| 589 | alpha-2-macroglobulin | 71321 | 5.47 | 1% |
| 589 | creatine kinase M | 43247 | 6.63 | 3% |
| 589 | cAMP-specific phosphodiesterase PDE4D5 | 84945 | 5.03 | 1% |
| 589 | ceruloplasmin | 98321 | 5.29 | 1% |
| 589 | PREDICTED: similar to epsilon-COP protein isoform 6 | 37072 | 5.00 | 3% |
| | [Pan troglodytes] | | | |
| 589 | CTCL tumor antigen se2-1 | 94014 | 5.37 | 3% |
| 589 | aggrecan 1 | 251979 | 4.10 | 0% |
| 597 | carboxypeptidase N precursor | 61433 | 5.72 | 20% |
| 597 | plasma protease (C1) inhibitor precursor | 55375 | 6.09 | 18% |
| 597 | Cartilage oligomeric matrix protein precursor (COMP) | 85403 | 4.34 | 16% |
| 597 | putative | 2269 | 9.79 | 38% |
| 597 | Aggrecan core protein precursor (Cartilage-specific | 251979 | 4.10 | 1% |
| | proteoglycan core protein) (CSPCP) (Chondroitin sulfate | | | |
| | proteoglycan core protein 1) [Contains: Aggrecan core | | | |
| | protein 2] | | | |
| 597 | alpha-1-antichymotrypsin | 48834 | 5.79 | 12% |
| 597 | cadherin 13 preproprotein | 78694 | 4.80 | 1% |
| 597 | Lumican | 38717 | 6.16 | 15% |
| 597 | dermcidin preproprotein | 11391 | 6.08 | 10% |
| 597 | protein, alpha1 acid glyco | 21443 | 5.09 | 4% |
| 597 | creatine kinase M | 43247 | 6.63 | 1% |
| 597 | apolipoprotein D, apoD [human, plasma, Peptide, 246 aa] | 28317 | 5.14 | 4% |
| 597 | CTCL tumor antigen se2-1 | 94014 | 5.37 | 3% |
| 597 | hCG2038603, isoform CRA_a | 9461 | 9.76 | 8% |
| 597 | B-cell receptor-associated protein BAP29 | 28512 | 9.63 | 6% |
| 597 | cAMP-specific phosphodiesterase PDE4D5 | 84945 | 5.03 | 1% |
| 600 | plasma protease (C1) inhibitor precursor | 55375 | 6.09 | 13% |
| 600 | PREDICTED: similar to Carboxypeptidase N subunit 2 | 61401 | 5.72 | 20% |
| | precursor (Carboxypeptidase N polypeptide 2) | | | |
| 600 | Cartilage oligomeric matrix protein precursor (COMP) | 85403 | 4.34 | 9% |
| 600 | alpha-1-antichymotrypsin | 48834 | 5.79 | 16% |
| 600 | lumican | 38717 | 6.16 | 15% |
| 600 | putative | 2269 | 9.79 | 38% |
| 600 | apolipoprotein D, apoD [human, plasma, Peptide, 246 aa] | 28317 | 5.14 | 10% |
| 600 | Aggrecan core protein precursor (Cartilage-specific | 251979 | 4.10 | 0% |
| | proteoglycan core protein) | | | |
| 600 | dermcidin preproprotein | 11391 | 6.08 | 10% |
| 600 | cadherin 13 preproprotein | 78694 | 4.80 | 2% |
| 600 | protein, alpha1 acid glyco | 21443 | 5.09 | 4% |
| 600 | hCG2038603, isoform CRA_a | 9461 | 9.76 | 8% |
| 600 | creatine kinase M | 43247 | 6.63 | 3% |
| 600 | fibronectin precursor | 260064 | 5.45 | 0% |
| 600 | PREDICTED: similar to Cyclin G-associated kinase | 148776 | 9.19 | 2% |
| 604 | alpha-2-macroglobulin precursor | 163175 | 6.00 | 9% |
| 604 | fibrinogen gamma | 46252 | 5.54 | 22% |
| 604 | complement C1r subcomponent precursor | 80122 | 5.89 | 15% |
| 604 | complement C4B precursor | 188230 | 7.39 | 5% |
| 604 | ceruloplasmin | 97637 | 5.29 | 3% |
| 610 | Alpha-2-macroglobulin precursor | 164600 | 5.50 | 2% |
| 610 | complement component C4A | 194337 | 6.00 | 13% |
| 610 | complement component C4 | 194216 | 6.65 | 7% |
| 610 | complement C4d | 31323 | 6.89 | 7% |
| 610 | ceruloplasmin | 116197 | 4.72 | 21% |
| 610 | afamin precursor | 70963 | 5.43 | 10% |
| 610 | fibrinogen gamma chain | 50077 | 5.64 | 17% |
| 610 | complement component C3 | 188585 | 5.61 | 9% |
| 610 | complement C1r subcomponent precursor | 81661 | 6.02 | 1% |
| 610 | inter-alpha-trypsin inhibitor C-terminal | 93745 | 5.89 | 7% |
| 610 | regucalcin | 33802 | 6.02 | 4% |
| 610 | trypsin inhibitor | 107103 | 5.89 | 5% |
| 610 | cardiotrophin-like cytokine factor 1 | 25388 | 6.58 | 6% |
| 610 | KIAA0805 | 150569 | 8.68 | 3% |
| 611 | complement component C4A | 194337 | 6.65 | 5% |
| 611 | alpha 2 macroglobulin | 167505 | 6.06 | 9% |
| 611 | inter-alpha-trypsin inhibitor | 103549 | 6.51 | 5% |
| 611 | FAM124A | 32947 | 5.47 | 1% |
| 611 | ceruloplasmin | 98321 | 5.29 | 1% |
| 611 | L-serine dehydrase | 35079 | 8.11 | 2% |
| 611 | hCG2032446 | 4973 | 10.15 | 18% |
| 612 | Alpha-2-macroglobulin precursor (Alpha-2-M) | 164600 | 6.00 | 14% |
| 612 | complement C4B precurso | 189599 | 7.39 | 6% |
| 612 | fibrinogen gamma chain | 50077 | 5.61 | 8% |
| 612 | Complement C1r subcomponent precursor (Complement | 81661 | 5.89 | 10% |
| | component 1, r subcomponent) | | | |
| 612 | putative | 2269 | 9.79 | 38% |
| 612 | ceruloplasmin | 98321 | 5.29 | 3% |
| 612 | DEP domain containing 1, isoform CRA_c | 84786 | 8.71 | 0% |
| 612 | membrane metallo-endopeptidase-like 1 | 89152 | 5.63 | 2% |
| 616 | alpha-2-macroglobulin precursor | 163175 | 6.00 | 19% |
| 616 | complement C4B precursor | 188230 | 7.36 | 12% |
| 616 | fibrinogen gamma chain | 49450 | 5.61 | 12% |
| 616 | complement C1r subcomponent precursor | 80122 | 5.89 | 17% |
| 616 | ceruloplasmin | 97637 | 5.29 | 10% |
| 616 | inter-alpha-trypsin C-terminal | 825681 | 6.02 | 6% |
| 616 | complement component C3 | 187046 | 6.02 | 3% |
| 616 | regucalcin | 33231 | 5.89 | 5% |
| 616 | Chain G, Gelsolin G4-G6ACTIN COMPLEX | 36347 | 5.00 | 13% |
| 616 | ANXA2 protein | 40503 | 8.41 | 14% |
| 616 | afamin precursor | 69024 | 5.64 | 4% |
| 616 | trypsin inhibitor | 106647 | 6.58 | 4% |
| 616 | hemopexin | 13338 | 6.70 | 9% |
| 616 | C1-inhibitor | 32688 | 8.85 | 2% |
| 616 | SMF protein | 156810 | 7.48 | 1% |
| 616 | alpha-2-antiplasmin precursor | 54194 | 5.71 | 2% |
| 622 | gelsolin isoform a precursor | 86043 | 5.90 | 16% |
| 622 | putative | 2269 | 9.79 | 38% |
| 622 | Alpha-2-macroglobulin precursor (Alpha-2-M) | 164600 | 6.00 | 4% |
| 622 | inter-alpha-trypsin inhibitor C-terminal | 93745 | 6.02 | 3% |
| 622 | annexin A2 isoform 2 | 38808 | 7.57 | 13% |
| 622 | dermcidin preproprotein | 11391 | 6.08 | 10% |
| 622 | Chain A, Structure Of Human Trypsin Iv (Brain Trypsin) | 24832 | 6.56 | 5% |
| 622 | unnamed protein product | 7974 | 4.93 | 13% |
| 622 | glucosamine (N-acetyl)-6-sulfatase precursor | 62840 | 8.60 | 2% |
| 622 | hCG2040015, isoform CRA_a | 47466 | 6.07 | 1% |
| 622 | Complement C1r subcomponent precursor (Complement | 81661 | 5.89 | 15% |
| | component 1, r subcomponent) | | | |
| 622 | potassium voltage-gated channel, shaker-related subfamily, | 39547 | 8.81 | 1% |
| | beta member 2 isoform 2 | | | |
| 652 | inter-alpha-trypsin inhibitor heavy chain H2 precursor | 106826 | 6.40 | 11% |
| 652 | inter-alpha-trypsin inhibitor family heavy chain-related | 103549 | 6.51 | 8% |
| | protein | | | |
| 652 | unnamed protein product | 70723 | 5.53 | 18% |
| 652 | complement component 1 | 78174 | 4.86 | 15% |
| 652 | vitamin K-dependent protein | 77127 | 5.48 | 13% |
| 652 | inter-alpha-trypsin inhibitor heavy chain H3 precursor | 99401 | 5.61 | 8% |
| 652 | afamin precursor | 70963 | 5.64 | 13% |
| 652 | ASPIC | 71448 | 4.98 | 10% |
| 652 | phospholipid transfer protein | 44989 | 8.69 | 11% |
| 652 | inter-alpha-trypsin inhibitor | 93745 | 6.02 | 6% |
| 652 | putative | 2269 | 9.79 | 38% |
| 652 | lumican | 38717 | 6.16 | 18% |
| 652 | dermcidin preproprotein | 11391 | 6.08 | 10% |
| 652 | Cartilage oligomeric matrix protein precursor (COMP) | 85403 | 4.34 | 1% |
| 652 | gelsolin isoform a precursor | 52511 | 5.21 | 2% |
| 652 | C1-inhibitor | 32745 | 8.85 | 3% |
| 652 | cAMP-specific phosphodiesterase PDE4D5 | 84945 | 5.03 | 1% |
| 652 | coagulation factor XII | 68618 | 7.94 | 2% |
| 652 | proapolipoprotein | 28944 | 5.45 | 4% |
| 652 | TERF1 (TRF1)-interacting nuclear factor 2 | 39779 | 9.30 | 3% |
| 653 | fibrinogen gamma chain | 50077 | 5.61 | 13% |
| 653 | inter-alpha-trypsin inhibitor | 93745 | 6.02 | 5% |
| 653 | complement component C3 | 188585 | 6.02 | 1% |
| 653 | KIAA1481 | 150746 | 6.77 | 0% |
| 653 | glucosamine (N-acetyl)-6-sulfatase precursor | 62840 | 8.60 | 2% |
| 654 | complement component 1 | 78174 | 4.86 | 14% |
| 654 | mutant beta-actin | 42128 | 5.22 | 6% |
| 654 | ASPIC | 71448 | 4.98 | 4% |
| 654 | lumican | 38717 | 6.16 | 12% |
| 654 | complement component C3 | 188585 | 6.02 | 2% |
| 654 | inter-alpha-trypsin inhibitor | 99401 | 5.61 | 4% |
| 654 | afamin precursor | 70963 | 5.64 | 4% |
| 654 | trypsin inhibitor | 107103 | 6.58 | 3% |
| 663 | plasma protease (C1) inhibitor precursor | 55375 | 6.09 | 31% |
| 663 | putative | 2269 | 9.79 | 38% |
| 663 | complement component C3 | 188585 | 6.02 | 0% |
| 663 | Aggrecan core protein precursor (Cartilage-specific | 251979 | 4.10 | 1% |
| | proteoglycan core protein) | | | |
| 663 | protein, alpha1 acid glyco | 21443 | 5.09 | 4% |
| 663 | unnamed protein product | 11786 | 5.80 | 0% |
| 663 | lumican | 38717 | 6.16 | 8% |
| 663 | ataxia-telangiectasia | 355538 | 6.37 | 1% |
| 663 | KIAA0056 | 171834 | 7.55 | 2% |
| 664 | no significant hits to report | 0 | 0.00 | 0% |
| 665 | gelsolin isoform a precursor | 85644 | 5.90 | 37% |
| 665 | unnamed protein product | 7974 | 5.48 | 34% |
| 665 | Complement C1r subcomponent precursor (Complement | 80122 | 5.89 | 4% |
| | component 1, r subcomponent) | | | |
| 665 | fibrinogen gamma chain, isoform CRA_o | 47344 | 5.54 | 10% |
| 665 | hemopexin, isoform CRA_c | 28546 | 6.55 | 10% |
| 665 | Trypsin-3 precursor | 32478 | 7.46 | 10% |
| 665 | dimethylglycine dehydrogenase-like protein isoform 2; | 51949 | 6.86 | 9% |
| | putative sarcosine dehydrogenase | | | |
| 673 | Inter-alpha-trypsin inhibitor heavy chain H2 precursor (ITI | 106826 | 6.40 | 18% |
| | heavy chain H2) | | | |
| 673 | inter-alpha-trypsin inhibitor family heavy chain-related | 103549 | 6.51 | 15% |
| | protein (IHRP) | | | |
| 673 | unnamed protein product | 70723 | 5.53 | 14% |
| 673 | Vitamin K-dependent protein S precursor | 77127 | 5.48 | 9% |
| 673 | afamin precursor | 70963 | 5.64 | 7% |
| 673 | putative | 2269 | 9.79 | 38% |
| 673 | Inter-alpha-trypsin inhibitor heavy chain H1 precursor (ITI | 1011782 | 6.31 | 3% |
| | heavy chain H1) | | | |
| 673 | caspase 14 precursor | 27947 | 5.44 | 4% |
| 673 | KIAA1481 protein | 150746 | 6.77 | 0% |
| 673 | Plasminogen | 93263 | 6.89 | 7% |
| 673 | creatine kinase M | 43247 | 6.63 | 3% |
| 673 | cAMP-specific phosphodiesterase PDE4D5 | 84945 | 5.03 | 1% |
| 676 | afamin | 69024 | 5.64 | 41% |
| 676 | inter-alpha-trypsin inhibitor heavy chain H2 precursor | 106370 | 6.40 | 20% |
| 676 | inter-alpha (globulin) inhibitor H1 | 101339 | 6.31 | 10% |
| 676 | fibulin | 61552 | 4.85 | 9% |
| 676 | inter-alpha-trypsin inhibitor family heavy chain-related | 103321 | 6.51 | 5% |
| | protein (IHRP) | | | |
| 676 | complement C4B precursor | 188230 | 7.39 | 7% |
| 676 | unnamed protein product | 69241 | 5.53 | 10% |
| 676 | complement component C | 187046 | 6.02 | 2% |
| 676 | ceruloplasmin | 97637 | 5.29 | 7% |
| 676 | alpha-1-B-glycoprotein | 69990 | 5.65 | 8% |
| 676 | unnamed protein product | 78029 | 8.51 | 2% |
| 676 | lacritin | 14237 | 5.45 | 16% |
| 676 | regucalcin | 33231 | 5.89 | 5% |
| 676 | C9 complement protein | 62974 | 5.49 | 5% |
| 676 | lipophilin | 9891 | 9.41 | 10% |
| 676 | lipocalin 1 precursor | 19238 | 5.39 | 6% |
| 676 | secretoglobin, family 2A, member 1 | 10876 | 5.48 | 43% |
| 676 | Complement C5 precursor [Contains: Complement C5 beta | 188212 | 6.11 | 0% |
| | chain; Complement C5 alpha chain; C5a anaphylatoxin; | | | |
| | Complement C5 alpha′ chain] | | | |
| 676 | proapolipoprotein | 28944 | 5.45 | 4% |
| 676 | hemopexin | 13338 | 6.70 | 9% |
| 676 | alpha-2-antiplasmin precursor | 54194 | 5.71 | 7% |
| 676 | regulator of G-protein signaling 9 | 76917 | 9.42 | 8% |
| 676 | lumican | 38375 | 6.16 | 14% |
| 679 | inter-alpha-trypsin inhibitor heavy chain H2 precursor | 106826 | 6.40 | 22% |
| 679 | coagulation factor II precursor | 71475 | 5.64 | 35% |
| 679 | afamin precursor | 70963 | 5.64 | 30% |
| 679 | inter-alpha-trypsin inhibitor family heavy chain-related | 103549 | 6.51 | 11% |
| | protein | | | |
| 679 | heat shock protein 90 kDa alpha (cytosolic) | 98622 | 5.09 | 18% |
| 679 | lumican | 38717 | 6.16 | 28% |
| 679 | vitamin k-dependent protein S precursor | 77127 | 5.48 | 11% |
| 679 | inter-alpha-trypsin inhibitor C-terminal | 93745 | 6.02 | 6% |
| 679 | fibulin-1A | 65487 | 4.85 | 5% |
| 679 | coagulation factor XII | 68618 | 7.94 | 4% |
| 679 | regucalcin | 33802 | 5.89 | 5% |
| 679 | complement component C3 | 188585 | 6.02 | 2% |
| 679 | phospholipid transfer protein isoform a precursor | 54933 | 6.53 | 6% |
| 679 | C4B3 | 47973 | 5.78 | 7% |
| 679 | ASPIC | 71448 | 4.98 | 1% |
| 679 | ceruloplasmin | 98321 | 5.29 | 1% |
| 679 | gelsolin isoform a precursor | 86043 | 5.90 | 1% |
| 679 | unnamed protein product | 79911 | 8.51 | 1% |
| 679 | cardiotrophin-like cytokine factor 1 | 25388 | 8.68 | 3% |
| 688 | afamin precursor | 69024 | 5.64 | 44% |
| 688 | Inter-alpha-trypsin inhibitor heavy chain H2 precursor | 106370 | 6.40 | 21% |
| 688 | inter-alpha (globulin) inhibitor H1 | 101339 | 6.31 | 10% |
| 688 | fibulin-1A | 61552 | 4.85 | 12% |
| 688 | unnamed protein product | 69241 | 5.53 | 13% |
| 688 | Chain B, Structure Of Complement C3b | 103886 | 5.18 | 8% |
| 688 | Ceruloplasmin | 97637 | 5.29 | 5% |
| 688 | Vitamin K-dependent protein S precursor | 75074 | 5.48 | 6% |
| 688 | lumican | 38375 | 6.16 | 18% |
| 688 | inter-alpha-trypsin inhibitor family heavy chain-related | 103321 | 6.51 | 5% |
| | protein (IHRP) | | | |
| 688 | complement component C4A | 192741 | 6.65 | 2% |
| 688 | precursor of P100 serine protease of Ra-reactive factor | 79210 | 5.42 | 4% |
| 688 | serpin peptidase inhibitor, clade I (pancpin), member 2 | 46116 | 5.08 | 5% |
| 688 | butyrylcholinesterase precursor | 68374 | 7.12 | 3% |
| 688 | annexin A2 isoform 2 | 38580 | 7.57 | 2% |
| 688 | phospholipid transfer protein isoform a precursor | 54705 | 6.53 | 5% |
| 688 | alpha-1-B-glycoprotein - human | 51908 | 5.65 | 2% |
| 688 | gelsolin isoform a precursor | 85644 | 5.90 | 2% |
| 688 | 90 kDa heat shock protein | 83242 | 4.97 | 3% |
| 688 | dermcidin preproprotein | 11277 | 6.08 | 10% |
| 688 | cAMP-specific phosphodiesterase PDE4D5 | 84375 | 5.03 | 1% |
| 695 | fibrinogen gamma chain | 50077 | 5.54 | 26% |
| 695 | Chain B, Human Complement Component C3 | 114238 | 5.50 | 10% |
| 695 | inter-alpha-trypsin inhibitor C-terminal | 93745 | 6.02 | 6% |
| 695 | glucosamine (N-acetyl)-6-sulfatase precursor | 62840 | 8.60 | 2% |
| 695 | hemopexin | 13452 | 6.70 | 9% |
| 695 | alpha-2-macroglobulin precursor | 164600 | 6.00 | 1% |
| 695 | complement C1r subcomponent precursor | 81661 | 5.89 | 6% |
| 697 | trypsin inhibitor | 107103 | 6.58 | 13% |
| 697 | afamin precursor | 70963 | 5.64 | 27% |
| 697 | complement component C3 | 188585 | 6.02 | 8% |
| 697 | Chain A, Crystal Structure Of Lipid-Free Human | 28061 | 5.27 | 39% |
| | Apolipoprotein A-I | | | |
| 697 | Inter-alpha-trypsin inhibitor heavy chain H1 precursor (ITI | 101782 | 6.31 | 9% |
| | heavy chain H1) (Inter-alpha-inhibitor heavy chain 1) | | | |
| | (Inter-alpha-trypsin inhibitor complex component III) | | | |
| | (Serum-derived hyaluronan-associated protein) (SHAP) | | | |
| 697 | inter-alpha-trypsin inhibitor family heavy chain-related | 103549 | 6.51 | 9% |
| | protein (IHRP) | | | |
| 697 | putative | 2269 | 9.79 | 38% |
| 697 | insulin-like growth factor binding protein, acid labile | 66735 | 6.33 | 14% |
| | subunit | | | |
| 697 | antithrombin III | 53041 | 6.32 | 9% |
| 697 | ceruloplasmin | 116197 | 5.43 | 4% |
| 697 | dermcidin preproprotein | 11391 | 6.08 | 10% |
| 697 | complement C4B precursor | 189599 | 7.39 | 2% |
| 697 | lumican | 38717 | 6.16 | 10% |
| 697 | hemopexin precursor | 52254 | 6.57 | 4% |
| 697 | coagulation factor XII | 68618 | 7.94 | 2% |
| 697 | Chain L, Alpha-Thrombin (E.C.3.4.21.5) Complex With | 4145 | 4.65 | 27% |
| | Hirulog 3 | | | |
| 697 | Vitamin D-binding protein precursor (DBP) (Group-specific | 54526 | 5.40 | 4% |
| | component) (Gc-globulin) (VDB) | | | |
| 697 | C9 complement protein | 64399 | 5.49 | 2% |
| 697 | Pyruvate dehydrogenase E1 component subunit beta, | 39536 | 6.20 | 4% |
| | mitochondrial precursor (PDHE1-B) | | | |
| 697 | Iron-binding acute phase protein (88 kDa Eales protein) | 2956 | 4.22 | 28% |
| 697 | unnamed protein product | 79911 | 8.51 | 1% |
| 697 | alpha-1-B-glycoprotein - human | 52479 | 5.65 | 2% |
| 700 | inter-alpha (globulin) inhibitor H2 | 105150 | 6.56 | 23% |
| 700 | complement component 3 precursor | 187030 | 6.02 | 17% |
| 700 | afamin precursor | 69024 | 5.64 | 28% |
| 700 | inter-alpha-trypsin inhibitor family heavy chain-related | 103321 | 6.51 | 17% |
| | protein (IHRP) | | | |
| 700 | unnamed protein product | 69241 | 5.53 | 16% |
| 700 | poly-Ig receptor | 75474 | 5.38 | 6% |
| 700 | complement component C3b - human (fragments) | 25280 | 4.49 | 28% |
| 700 | Inter-alpha-trypsin inhibitor heavy chain H1 precursor (ITI | 101326 | 6.31 | 11% |
| | heavy chain H1) | | | |
| 700 | Chain A, Crystal Structure Of Lipid-Free Human | 28061 | 5.27 | 38% |
| | Apolipoprotein A-I | | | |
| 700 | coagulation factor XII | 66337 | 7.94 | 7% |
| 700 | ceruloplasmin | 115398 | 5.43 | 6% |
| 700 | insulin-like growth factor binding protein, acid labile | 65994 | 6.33 | 6% |
| | subunit | | | |
| 700 | complement C4B precursor | 188230 | 7.39 | 6% |
| 700 | Chain A, Apo-Human Serum Transferrin (Non- | 74643 | 6.58 | 8% |
| | Glycosylated) | | | |
| 700 | alpha-1-B-glycoprotein - human | 51908 | 5.65 | 11% |
| 700 | serum vitamin D-binding protein precursor | 53015 | 5.40 | 22% |
| 700 | hemopexin precursor | 51512 | 6.57 | 12% |
| 700 | histidine-rich glycoprotein precursor | 59541 | 7.09 | 4% |
| 703 | inter-alpha globulin inhibitor H2 polypeptide | 106397 | 6.40 | 21% |
| 703 | afamin precursor | 69024 | 5.64 | 21% |
| 703 | inter-alpha-trypsin inhibitor C-terminal | 93402 | 6.02 | 7% |
| 703 | inter-alpha-trypsin inhibitor family heavy chain-related | 103321 | 6.51 | 6% |
| | protein (IHRP) | | | |
| 703 | Chain A, Crystal Structure Of Lipid-Free Human | 28061 | 5.27 | 49% |
| | Apolipoprotein A-I | | | |
| 703 | ceruloplasmin | 97637 | 5.29 | 8% |
| 703 | Chain B, Structure Of Complement C3b: Insights Into | 103886 | 5.18 | 8% |
| | Complement Activation And Regulation | | | |
| 703 | unnamed protein product | 69241 | 5.53 | 8% |
| 703 | hemopexin, isoform CRA_a | 22935 | 6.78 | 9% |
| 703 | Chain A, Apo-Human Serum Transferrin (Non- | 74643 | 6.58 | 7% |
| | Glycosylated) | | | |
| 703 | insulin-like growth factor binding protein, acid labile | 65994 | 6.33 | 7% |
| | subunit | | | |
| 703 | fibrinogen gamma chain | 49450 | 5.61 | 6% |
| 703 | glucosamine (N-acetyl)-6-sulfatase precursor | 62042 | 8.60 | 2% |
| 703 | complement component C4A | 193541 | 6.79 | 3% |
| 703 | PREDICTED: similar to Prostate, ovary, testis expressed | 117315 | 5.66 | 3% |
| | protein on chromosome 2 | | | |
| 703 | phospholipid transfer protein isoform a precursor | 54705 | 6.53 | 6% |
| 709 | ASPIC | 71448 | 4.98 | 17% |
| 709 | inter-alpha (globulin) inhibitor H3 | 100072 | 5.53 | 6% |
| 709 | putative | 2269 | 9.79 | 38% |
| 709 | Chain H, Alpha-Thrombin (E.C.3.4.21.5) Complex With | 30118 | 8.88 | 4% |
| | Hirulog 3 | | | |
| 709 | coagulation factor XII | 68618 | 7.94 | 5% |
| 709 | phospholipid transfer protein isoform a precursor | 54933 | 6.53 | 3% |
| 709 | inter-alpha-trypsin inhibitor family heavy chain-related | 103549 | 6.51 | 2% |
| | protein (IHRP) | | | |
| 709 | lumican | 38717 | 6.16 | 21% |
| 709 | trypsin inhibitor | 107103 | 6.58 | 2% |
| 709 | afamin precursor | 70963 | 5.64 | 6% |
| 709 | dermcidin preproprotein | 11391 | 6.08 | 10% |
| 709 | biotinidase precursor | 62006 | 5.81 | 3% |
| 709 | filaggrin 2 | 249296 | 8.45 | 0% |
| 709 | creatine kinase M | 43247 | 6.63 | 3% |
| 709 | cAMP-specific phosphodiesterase PDE4D5 | 84945 | 5.03 | 2% |
| 709 | Interferon-induced guanylate-binding protein 2 (GTP- | 67654 | 5.54 | 4% |
| | binding protein 2) | | | |
| 710 | inter-alpha (globulin) inhibitor H1 | 101795 | 6.31 | 23% |
| 710 | inter-alpha-trypsin inhibitor heavy chain H2 | 106826 | 6.40 | 19% |
| 710 | insulin-like growth factor binding protein | 66735 | 6.33 | 20% |
| 710 | coagulation factor XII | 68618 | 7.94 | 8% |
| 710 | hemopexin precursor | 52254 | 6.57 | 13% |
| 710 | unnamed protein product | 70723 | 5.53 | 9% |
| 710 | histidine-rich glycoprotein precursor | 60510 | 7.09 | 9% |
| 710 | ceruloplasmin | 98321 | 5.29 | 7% |
| 710 | complement C4B precursor | 189599 | 7.39 | 2% |
| 710 | complement component C3 | 179665 | 6.02 | 1% |
| 710 | lumican | 38717 | 6.16 | 5% |
| 710 | glucosamine (N-acetyl)-6-sulfatase precursor | 62840 | 8.60 | 4% |
| 721 | Inter-alpha-trypsin inhibitor heavy chain H1 precursor (ITI | 101782 | 6.31 | 19% |
| | heavy chain H1) | | | |
| 721 | fibrinogen gamma | 46823 | 5.54 | 29% |
| 721 | glucosamine (N-acetyl)-6-sulfatase precursor | 62840 | 8.60 | 8% |
| 721 | putative | 62320 | 9.79 | 38% |
| 721 | Alpha-2-macroglobulin precursor (Alpha-2-M) | 164600 | 6.00 | 3% |
| 721 | annexin A2 isoform 2 | 38808 | 7.57 | 6% |
| 721 | DEP domain containing 1, isoform CRA_c | 84786 | 8.71 | 0% |
| 721 | histidine-rich glycoprotein precursor | 84768 | 7.09 | 1% |
| 721 | lymphoid-restricted membrane protein | 56691 | 5.44 | 1% |
| 722 | inter-alpha-trypsin inhibitor heavy chain H1 precursor | 101782 | 6.31 | 21% |
| 722 | fibrinogen gamma chain | 50077 | 5.61 | 29% |
| 722 | gamma-actin | 26147 | 5.65 | 18% |
| 722 | Na/K-ATPase alpha 1 subunit isoform a proprotein | 114135 | 5.33 | 10% |
| 722 | glucosamine (N-acetyl)-6-sulfatase precursor | 62840 | 8.60 | 8% |
| 722 | annexin A2 | 38808 | 7.57 | 6% |
| 722 | alpha-2-macroglobulin precursor | 164600 | 6.00 | 2% |
| 722 | hornerin | 48797 | 9.71 | 6% |
| 722 | heat shock protein gp96 precursor | 90309 | 4.73 | 6% |
| 722 | trypsin (EC 3.4.21.4) III precursor - human | 27329 | 5.95 | 9% |
| 722 | coagulation factor XII | 68618 | 7.94 | 7% |
| 722 | sodium/potassium-transporting ATPase alpha-4 chain | 115119 | 6.23 | 4% |
| 722 | filaggrin 2 | 249296 | 8.45 | 0% |
| 722 | alpha 1 actin precursor | 42366 | 5.23 | 6% |
| 722 | 4F2 heavy chain antigen | 58155 | 5.15 | 2% |
| 722 | complement component C3 | 188585 | 6.02 | 1% |
| 722 | histidine-rich glycoprotein precursor | 60510 | 7.09 | 1% |
| 722 | ATP synthase, H transporting, mitochondrial | 59828 | 9.16 | 2% |
| 722 | cAMP-specific phosphodiesterase PDE4D5 | 84945 | 5.03 | 1% |
| 727 | ASPIC | 71448 | 4.98 | 33% |
| 727 | coagulation factor XII | 68618 | 7.94 | 12% |
| 727 | lumican | 38717 | 6.16 | 18% |
| 727 | hornerin | 48797 | 9.71 | 10% |
| 727 | inter-alpha-trypsin inhibitor | 103549 | 6.51 | 5% |
| 727 | inter-alpha-trypsin inhibitor heavy chain H3 precursor | 99401 | 5.61 | 4% |
| 727 | unnamed protein | 70723 | 8.27 | 0% |
| 727 | histidine-rich glycoprotein precursor | 60510 | 7.09 | 5% |
| 730 | inter-alpha (globulin) inhibitor H1 | 101339 | 6.31 | 19% |
| 730 | inter-alpha-trypsin inhibitor heavy chain H2 | 106370 | 6.40 | 12% |
| 730 | glucosamine (N-acetyl)-6-sulfatase precursor | 62042 | 8.60 | 12% |
| 730 | complement conponent C3 | 187046 | 6.02 | 7% |
| 730 | fibrinogen gamma chain | 49450 | 5.61 | 15% |
| 730 | afamin precursor | 69024 | 5.64 | 12% |
| 730 | glycoprotein V (platelet) | 60921 | 9.73 | 4% |
| 730 | annexin A2 isoform 2 | 38580 | 7.57 | 5% |
| 730 | alpha-2-macroglobulin precursor | 163175 | 6.00 | 2% |
| 730 | TPA: Hornerin | 282204 | 10.04 | 2% |
| 730 | plasminogen | 90526 | 6.89 | 3% |
| 730 | Beta-2-glycoprotein 1 precursor (Beta-2-glycoprotein I) | 38273 | 8.34 | 4% |
| | (Apolipoprotein H) | | | |
| 730 | inter-alpha-trypsin inhibitor family heavy chain-related | 103321 | 6.51 | 1% |
| | protein (IHRP) | | | |
| 743 | ASPIC | 71448 | 4.98 | 30% |
| 743 | coagulation factor XII | 68618 | 7.94 | 7% |
| 743 | inter-alpha-trypsin family heavy chain-related protein | 103549 | 6.51 | 4% |
| 743 | inter-alpha-trypsin heavy chain H3 precursor | 99401 | 5.61 | 7% |
| 743 | lumican | 38717 | 6.16 | 16% |
| 743 | unnamed protein (vitronectin) | 55106 | 5.55 | 7% |
| 743 | complement component C3 | 188585 | 6.02 | 0% |
| 743 | DEP domain containing 1, isoform | 84786 | 8.71 | 0% |
| 787 | complement component C3 | 188585 | 6.02 | 16% |
| 787 | phospholipid transfer protein | 44989 | 8.69 | 20% |
| 787 | hemopexin precursor | 52254 | 6.57 | 14% |
| 787 | Chain I, Crystal Structure Of P13 Alanine Variant Of | 49276 | 6.13 | 20% |
| | Antithrombin | | | |
| 787 | ceruloplasmin | 98321 | 5.29 | 7% |
| 787 | alpha-1-B-glycoprotein | 52479 | 5.65 | 17% |
| 787 | putative | 54809 | 9.79 | 38% |
| 787 | histidine-rich glycoprotein precursor | 60510 | 7.09 | 10% |
| 787 | inter-alpha-trypsin inhibitor C-terminal | 93745 | 6.02 | 1% |
| 787 | proapolipoprotein | 28944 | 5.45 | 4% |
| 787 | Chain L, Alpha-Thrombin (E.C.3.4.21.5) Complex With | 4145 | 4.65 | 27% |
| | Hirulog 3 | | | |
| 787 | Chain A, Crystal Structure Of Native Heparin Cofactor Ii | 55096 | 6.26 | 3% |
| 787 | JAK family protein tyrosine kinase | 126761 | 6.64 | 5% |
| 787 | cAMP-specific phosphodiesterase PDE4D5 | 84945 | 5.03 | 1% |
| 805 | alpha 2 macroglobulin | 166022 | 6.06 | 2% |
| 805 | anti-colorectal carcinoma heavy chain | 50570 | 6.22 | 2% |
| 805 | zinc finger protein 217 | 115199 | 8.78 | 1% |
| 817 | Chain B, Structure Of Complement C3b: Insights Into | 104912 | 5.18 | 27% |
| | Complement Activation And Regulation | | | |
| 817 | alpha-1-B-glycoprotein - human | 52479 | 5.65 | 20% |
| 817 | complement component C3b | 26135 | 4.49 | 21% |
| 817 | hemopexin precursor | 52254 | 6.57 | 14% |
| 817 | Complement C5 precursor [Contains: Complement C5 beta | 189923 | 6.11 | 1% |
| | chain; Complement C5 alpha chain; C5a anaphylatoxin; | | | |
| 817 | putative | 2269 | 9.79 | 38% |
| 817 | C9 complement protein | 64399 | 5.49 | 10% |
| 817 | alpha-2-antiplasmin precursor | 54536 | 5.71 | 7% |
| 817 | ceruloplasmin | 98321 | 5.29 | 3% |
| 817 | hyaluronan binding protein 2 | 64740 | 6.09 | 9% |
| 817 | dermcidin preproprotein | 11391 | 6.08 | 10% |
| 817 | Chain E, Prethrombin2 (E.C.3.4.21.5) Complexed With | 34245 | 8.32 | 7% |
| | Hirugen (N-Acetylhirudin 53-64 With Sulfato-Tyr 63) | | | |
| 817 | complement C4B precursor | 189599 | 7.39 | 2% |
| 817 | collagenase type IV precursor | 73279 | 5.20 | 3% |
| 817 | antithrombin III | 53041 | 6.32 | 8% |
| 817 | histidine-rich glycoprotein precursor | 60510 | 7.09 | 3% |
| 817 | ASPIC | 71448 | 4.98 | 2% |
| 817 | alpha 2-plasmin inhibitor, alpha 2-PI {N-terminal, form A} | 2064 | 4.53 | 63% |
| | [human, plasma, Peptide Partial, 19 aa] | | | |
| 817 | lumican | 38717 | 6.16 | 2% |
| 822 | alpha-1-B-glycoprotein | 52479 | 5.65 | 35% |
| 822 | Chain B, Structure of Complement C3b: . . . | 104912 | 5.18 | 25% |
| 822 | Chain A, Gelatinase A (full length) | 71842 | 5.20 | 20% |
| 822 | C9 complement protein | 64399 | 5.49 | 14% |
| 822 | hemopexin precursor | 52254 | 6.57 | 16% |
| 822 | histidine-rich glycoprotein precursor | 60510 | 7.09 | 9% |
| 822 | complement component 5 variant | 124357 | 8.43 | 3% |
| 822 | lumican | 38717 | 6.16 | 8% |
| 822 | alpha-2-antiplasmin precursor | 54536 | 5.71 | 10% |
| 822 | complement C4B precursor | 189599 | 7.39 | 2% |
| 822 | vitamin K-dependent protein S precursor | 77127 | 5.48 | 2% |
| 822 | ASPIC | 71448 | 4.98 | 1% |
| 825 | chain B, structure of complement C3b | 103886 | 5.18 | 33% |
| 825 | alpha-1-B-glycoprotein | 51908 | 5.65 | 22% |
| 825 | complement component 5 variant | 123274 | 8.43 | 9% |
| 825 | complement component 9 | 63133 | 5.43 | 27% |
| 825 | collagenase type IV precursor | 72196 | 5.20 | 18% |
| 825 | hemopexin precursor | 51512 | 6.57 | 16% |
| 825 | hornerin precursor | 282199 | 10.04 | 5% |
| 825 | complement C4B precursor | 188230 | 7.39 | 7% |
| 825 | SERPINF2 protein | 54559 | 5.87 | 19% |
| 825 | ceruloplasmin | 97637 | 5.29 | 4% |
| 825 | histidine-rich glycoprotein precursor | 59541 | 7.09 | 6% |
| 825 | afamin precursor | 69024 | 5.64 | 7% |
| 825 | ASPIC | 70650 | 4.98 | 7% |
| 825 | phospholipid transfer protein isoform a precursor | 54705 | 6.53 | 9% |
| 825 | hyaluronan binding protein 2 | 62630 | 6.09 | 8% |
| 825 | Vitamin K-dependent protein S precursor | 75074 | 5.48 | 6% |
| 825 | Chain A, Antithrombin Iii | 49008 | 5.95 | 15% |
| 825 | thrombin inhibitor | 42559 | 5.33 | 3% |
| 834 | Chain b, structure of complement C3b | 103886 | 5.18 | 34% |
| 834 | alpha-1-B-glycoprotein | 51908 | 5.65 | 22% |
| 834 | complement component 8, alpha polypeptide | 65121 | 6.07 | 14% |
| 834 | hemopexin precursor | 51512 | 6.57 | 31% |
| 834 | complement component C4A | 193541 | 6.79 | 6% |
| 834 | coagulation facotr II precursor | 69992 | 5.64 | 16% |
| 834 | ceruloplasmin | 97637 | 5.29 | 9% |
| 834 | hyalluronan binding protein 2 | 62630 | 6.09 | 11% |
| 834 | alpha-2-antiplasmin precursor | 54194 | 5.71 | 10% |
| 834 | heparin cofactor II precursor | 57062 | 6.41 | 13% |
| 834 | histidine-rich glycoprotein precursor | 59541 | 7.09 | 7% |
| 834 | unnamed protein product | 66412 | 5.62 | 9% |
| 834 | Complement C5 precursor | 188212 | 6.11 | 1% |
| 834 | ASPIC | 70650 | 4.98 | 5% |
| 846 | DEP domain containing 1, isoform CRA_c | 84786 | 8.71 | 0% |
| 846 | putative | 2269 | 9.79 | 38% |
| 859 | Chain b, Structure of Complement C3b | 104912 | 5.18 | 41% |
| 859 | alpha-1-B-glycoprotein | 52479 | 5.65 | 31% |
| 859 | hemopexin precursor | 52254 | 6.57 | 29% |
| 859 | antithrombin III | 53041 | 6.32 | 14% |
| 859 | hyaluronan binding protein 2 | 64740 | 6.09 | 3% |
| 859 | mutant beta-actin | 42128 | 5.22 | 4% |
| 859 | complement component C4A | 194337 | 6.65 | 0% |
| 859 | ceruloplasmin | 98321 | 5.29 | 1% |
| 859 | histidine-rich glycoprotein precursor | 60510 | 7.09 | 2% |
| 859 | hCG039828, isoform CRA_b | 10319 | 11.25 | 9% |
| 884 | complement 9 | 61728 | 5.42 | 33% |
| 884 | alpha-2-antiplasmin precursor | 54536 | 5.71 | 18% |
| 884 | Complement C5 precursor | 189923 | 6.11 | 6% |
| 884 | complement component 1 | 54192 | 6.75 | 9% |
| 884 | PRSS3 protein | 27040 | 6.86 | 8% |
| 884 | Alpha-2-macroglobulin precursor | 164600 | 6.00 | 2% |
| 884 | kininogen 1 | 48936 | 6.29 | 19% |
| 884 | alpha 2-plasmin inhibitor | 2064 | 4.53 | 63% |
| 884 | hemopexin precursor | 52254 | 6.57 | 12% |
| 884 | complement component C3 | 188585 | 6.02 | 2% |
| 884 | lumican | 38717 | 6.16 | 16% |
| 884 | Thyroxine-binding globulin precursor | 46637 | 5.87 | 8% |
| 884 | dermcidin preproprotein | 11391 | 6.08 | 10% |
| 884 | complement component C4A | 194337 | 6.65 | 2% |
| 884 | angiotensinogen | 53407 | 5.78 | 3% |
| 884 | apolipoprotein E | 36302 | 5.65 | 2% |
| 885 | complement component 4 binding protein | 69042 | 7.15 | 9% |
| 885 | unnamed protein (serum albumin precursor) | 71246 | 5.92 | 7% |
| 885 | trypsin inhibitor | 107103 | 6.58 | 3% |
| 885 | hemopexin precursor | 52254 | 6.57 | 95% |
| 885 | Chain A, Trypsin (transmembrane protease) | 24669 | 7.64 | 3% |
| 886 | alpha-1-antichymotrypsin precursor | 45567 | 5.32 | 6% |
| 886 | putative | 2269 | 9.79 | 38% |
| 886 | complement conponent C3 | 188585 | 6.02 | 2% |
| 886 | COMP_HUMAN | 91772 | 4.45 | 6% |
| 886 | lumican | 38717 | 6.16 | 10% |
| 886 | dermcidin preproprotein | 11391 | 6.08 | 10% |
| 886 | tumor endothelial marker 7-related precursor | 60116 | 5.99 | 1% |
| 886 | hCG22067 | 30319 | 7.89 | 3% |
| 886 | Plasminogen | 932663 | 6.89 | 1% |
| 886 | cAMP-specific phosphodiesterase PDE4D5 | 84945 | 5.03 | 1% |
| 887 | complement 9 | 61728 | 5.42 | 42% |
| 887 | complement C5 precursor | 189923 | 6.11 | 9% |
| 887 | alpha-2-antiplasmin precursor | 54536 | 5.71 | 31% |
| 887 | alpha-1-B-glycoprotein | 52479 | 5.65 | 28% |
| 887 | Chain A, antithrombin Iii | 49350 | 5.95 | 24% |
| 887 | complement component C3 | 188585 | 6.02 | 4% |
| 887 | hemopexin, isoform CRA_d | 43771 | 6.24 | 13% |
| 887 | hyaluronan binding protein 2 | 64740 | 6.09 | 3% |
| 887 | alpha-2-plasmin inhibitor | 2064 | 4.53 | 63% |
| 887 | ceruloplasmin | 116197 | 5.43 | 0% |
| 887 | Serpin B8 (cytoplasmic antiportease) | 43328 | 5.43 | 2% |
| 887 | T-plastin polypeptide | 64281 | 5.73 | 1% |
| 897 | hemopexin precursor | 52254 | 6.57 | 52% |
| 897 | putative | 2269 | 9.76 | 76% |
| 897 | DNA cytosine methyltransferase 3 alpha isoform a | 103390 | 6.19 | 0% |
| 897 | unnamed protein product | 112600 | 8.27 | 0% |
| 902 | complement 9 | 61728 | 5.42 | 38% |
| 902 | Complement C5 precursor | 189923 | 6.11 | 11% |
| 902 | alpha-2-antiplasmin precursor | 54536 | 5.71 | 34% |
| 902 | lumican | 38717 | 6.16 | 23% |
| 902 | complement component 1 | 54192 | 6.75 | 10% |
| 902 | hemopexin precursor | 52254 | 6.57 | 11% |
| 902 | alpha-2-plasmin inhibitor | 2064 | 4.53 | 63% |
| 902 | alpha-1-B-glycoprotein | 52479 | 5.65 | 3% |
| 902 | complement component C3 | 188585 | 6.02 | 0% |
| 903 | alpha-1-B-glycoprotein | 52479 | 5.65 | 40% |
| 903 | complement 9 | 61728 | 5.42 | 21% |
| 903 | L-plastin | 70815 | 5.20 | 26% |
| 903 | hyaluronan binding protein 2 | 64740 | 6.09 | 19% |
| 903 | Chain B, Structure of Complement C3b | 104912 | 5.18 | 19% |
| 903 | alpha-2-antiplasmin precursor | 54536 | 5.71 | 16% |
| 903 | Chain A, antithrombin Iii | 49350 | 5.95 | 31% |
| 903 | hemopexin precursor | 52254 | 6.57 | 24% |
| 903 | Complement C5 precursor | 189923 | 6.11 | 3% |
| 903 | alpha-2 plasma inhibitor | 2064 | 4.53 | 63% |
| 903 | complement component 1, r subcomponent-like precursor | 54192 | 6.75 | 2% |
| 903 | complement C4B precursor | 189599 | 7.39 | 7% |
| 903 | regucalcin | 33802 | 5.89 | 9% |
| 903 | lipoprotein Gln I | 28329 | 5.27 | 13% |
| 903 | PRSS3 | 27040 | 6.86 | 12% |
| 903 | ceruloplasmin | 98321 | 5.29 | 2% |
| 903 | heat shock 70 kDa protein 8 isoform 1 | 71082 | 5.37 | 3% |
| 903 | lumican | 38717 | 6.16 | 15% |
| 903 | c-AMP-specific phosphodiesterase PDE4DF | 84945 | 5.03 | 1% |
| 903 | hCG40021 | 114560 | 9.58 | 0% |
| 925 | Kininogen 1 | 47871 | 6.29 | 64% |
| 925 | lumican | 38375 | 6.16 | 39% |
| 925 | angiotensinogen | 53122 | 5.78 | 8% |
| 925 | unnamed protein product | 54308 | 5.55 | 10% |
| 925 | alpha-2-antiplasmin precursor | 54194 | 5.71 | 6% |
| 925 | complement C8-beta propetide /map = ‘1p36.2-p22.1’ | 62008 | 8.24 | 13% |
| | /hgml_locus_uid = ‘LE0166P’ | | | |
| 925 | dermcidin preproprotein | 11277 | 6.08 | 10% |
| 925 | alpha-1-antichymotrypsin precursor | 45453 | 5.32 | 11% |
| 925 | histidine-rich glycoprotein precursor | 59541 | 7.09 | 4% |
| 925 | apolipoprotein J precursor | 48772 | 6.27 | 4% |
| 925 | Thyroxine-binding globulin precursor (T4-binding globulin) | 46295 | 5.87 | 5% |
| | (Serpin A7) | | | |
| 925 | alpha-2-macroglobulin | 70751 | 5.47 | 5% |
| 925 | Plasminogen | 90526 | 6.89 | 4% |
| 925 | small proline-rich protein | 8049 | 9.07 | 25% |
| 925 | unnamed protein product | 129330 | 4.71 | 2% |
| 925 | antithrombin III | 52585 | 6.32 | 4% |
| 925 | ectonucleotide pyrophosphatase/phosphodiesterase 5 | 54631 | 5.94 | 2% |
| | (putative function) | | | |
| 925 | extracellular protein - human | 43109 | 5.26 | 5% |
| 925 | hemopexin precursor | 51512 | 6.57 | 8% |
| 925 | phosphodiesterase 4D, cAMP-specific (phosphodiesterase | 24635 | 9.88 | 12% |
| | E3 dunce homolog, Drosophila), isoform CRA_a | | | |
| 925 | unnamed protein product | 69250 | 5.92 | 5% |
| 934 | complement component 8 | 65121 | 6.21 | 39% |
| 934 | hemopexin precursor | 51512 | 6.57 | 30% |
| 934 | serum albumin precursor | 69180 | 5.91 | 31% |
| 934 | macroglobulin alpha2 | 160704 | 5.95 | 8% |
| 934 | leukotriene A4 hydrolase | 69241 | 5.80 | 7% |
| 934 | Plasminogen | 90526 | 6.89 | 4% |
| 937 | ASPIC | 71448 | 4.98 | 17% |
| 937 | Inter-alpha-trypsin inhibitor heavy chain H3 precursor (ITI | 99401 | 5.61 | 5% |
| | heavy chain H3) | | | |
| 937 | inter-alpha-trypsin inhibitor family heavy chain-related | 103549 | 6.51 | 2% |
| | protein (IHRP) | | | |
| 937 | lumican | 38717 | 6.16 | 21% |
| 937 | putative | 2269 | 9.79 | 38% |
| 937 | coagulation factor XII | 68618 | 7.94 | 25% |
| 937 | Chain H, Alpha-Thrombin (E.C.3.4.21.5) Complex With | 30118 | 8.88 | 10% |
| | Hirulog 3 | | | |
| 937 | phospholipid transfer protein isoform a precursor | 54933 | 6.53 | 3% |
| 937 | trypsin inhibitor | 107103 | 6.58 | 2% |
| 937 | biotinidase precursor | 62006 | 5.81 | 5% |
| 937 | filaggrin 2 | 249296 | 8.45 | 0% |
| 937 | inter-alpha-trypsin inhibitor C-terminal | 93745 | 6.02 | 35% |
| 937 | dermcidin preproprotein | 11391 | 6.08 | 10% |
| 937 | unnamed protein product | 55106 | 5.55 | 6% |
| 937 | creatine kinase M (EC 2.7.3.2) | 8024 | 6.63 | 3% |
| 937 | cAMP-specific phosphodiesterase PDE4D5 | 84945 | 5.03 | 1% |
| 937 | afamin precursor | 70963 | 5.64 | 4% |
| 937 | PREDICTED: similar to Cyclin G-associated kinase | 148776 | 9.19 | 4% |
| 937 | complement component 8, alpha polypeptide precursor | 66832 | 6.07 | 24% |
| 937 | serum albumin | 71316 | 6.05 | 30% |
| 937 | hemopexin precursor | 52254 | 6.57 | 23% |
| 937 | macroglobulin alpha2 | 162072 | 5.95 | 5% |
| 937 | putative | 2269 | 9.79 | 38% |
| 937 | kallistatin | 486966 | 7.33 | 10% |
| 937 | annexin A2 isoform 2 | 38808 | 7.57 | 11% |
| 937 | dermcidin preproprotein | 11391 | 6.08 | 10% |
| 937 | phosphodiesterase 4D, cAMP-specific (phosphodiesterase | 24806 | 9.88 | 12% |
| | E3 dunce homolog, Drosophila), isoform CRA_a | | | |
| 937 | lumican | 38717 | 6.16 | 7% |
| 937 | complement component C3 | 188585 | 6.02 | 1% |
| 937 | RNA polymerase II largest subunit | 218413 | 7.12 | 0% |
| 951 | hemopexin precursor | 52254 | 6.57 | 40% |
| 951 | complement component 8, polypeptide precursor | 66832 | 6.07 | 16% |
| 951 | alpha-2-macroglobulin precursor | 164600 | 6.00 | 4% |
| 951 | similar to human albumin, Swiss-Prot Accession Number | 53416 | 5.69 | 6% |
| | P02768 | | | |
| 951 | unnamed protein product | 112600 | 8.27 | 0% |
| 951 | putative | 2269 | 9.79 | 38% |
| 951 | DNA cytosine methyltransferase 3 alpha isoform a | 103390 | 6.19 | 1% |
| 951 | MDN1, midasin homolog | 638009 | 5.46 | 1% |
| 959 | hemopexin precursor | 52254 | 6.57 | 55% |
| 959 | alloalbumin Venezia | 71177 | 5.99 | 29% |
| 959 | complement component 8 | 66832 | 6.07 | 7% |
| 959 | chain A, complement component C2a | 58977 | 6.62 | 14% |
| 959 | alpha-2-macroglobulin precursor | 164600 | 6.00 | 5% |
| 959 | complement component C3 | 188585 | 6.02 | 7% |
| 959 | putative | 2269 | 9.79 | 38% |
| 959 | Plasminogen | 93263 | 6.89 | 2% |
| 959 | complement C8-beta propetide /map = ‘1p36.2-p22.1’ | 63605 | 8.24 | 1% |
| | /hgml_locus_uid = ‘LE0166P’ | | | |
| 959 | meltrin-beta/ADAM 19 homologue | 103606 | 8.62 | 3% |
| 959 | cAMP-specific phosphodiesterase PDE4D5 | 84945 | 5.03 | 1% |
| 959 | cardiotrophin-like cytokine factor 1 | 25388 | 8.68 | 3% |
| 959 | p107 | 122168 | 7.32 | 2% |
| 963 | beta-fibrinogen precursor | 55545 | 8.31 | 20% |
| 963 | alpha-2-macroglobulin precursor | 164600 | 6.00 | 8% |
| 963 | beta-2-glycoprotein precursor | 39584 | 8.34 | 13% |
| 963 | hemopexin precursor | 52254 | 6.57 | 8% |
| 963 | phospholipase D3 isoform 2 | 49196 | 6.00 | 6% |
| 963 | complement component 1 | 81788 | 5.89 | 10% |
| 963 | prolycarboxypeptidase | 56277 | 6.75 | 3% |
| 963 | annexin A2 | 38808 | 7.57 | 2% |
| 963 | regucalcin | 33802 | 5.89 | 5% |
| 963 | T-plastin polypeptide | 64281 | 5.73 | 7% |
| 968 | hemopexin precursor | 51512 | 6.57 | 46% |
| 968 | alpha 2 macroglobulin | 166022 | 6.06 | 9% |
| 968 | complement component 8, alpha polypeptide precursor | 65121 | 6.07 | 31% |
| 968 | serum albumin | 69321 | 6.05 | 33% |
| 968 | serine (or cysteine) proteinase inhibitor, clade A (alpha-1 | 48511 | 7.33 | 22% |
| | antiproteinase, antitrypsin), member 4 [ | | | |
| 968 | annexin A2 isoform 2 | 38580 | 7.57 | 29% |
| 968 | leukotriene A4 hydrolase | 69241 | 5.80 | 8% |
| 968 | regucalcin | 33231 | 5.86 | 5% |
| 968 | Collagen alpha-3(VI) chain precursor | 343340 | 6.40 | 3% |
| 968 | complement component C3 | 187046 | 6.02 | 1% |
| 968 | phospholipase D3 isoform 1 | 54671 | 6.02 | 5% |
| 968 | Plasminogen | 90526 | 6.89 | 2% |
| 968 | alpha-2-antiplasmin precursor | 54194 | 5.71 | 5% |
| 968 | Beta-2-glycoprotein 1 precursor (Beta-2-glycoprotein I) | 38273 | 8.34 | 11% |
| | (Apolipoprotein H) (Apo-H) (B2GPI) (Beta(2)GPI) | | | |
| | (Activated protein C-binding protein) (APC inhibitor) | | | |
| | (Anticardiolipin cofactor) | | | |
| 968 | major vault protein, isoform CRA_a | 17434 | 10.25 | 10% |
| 984 | alpha-1-antichymotrypsin precursor | 45567 | 5.32 | 36% |
| 984 | Corticosteroid-binding globulin precursor (CBG) | 45283 | 5.64 | 18% |
| | (Transcortin) (Serpin A6) | | | |
| 984 | unnamed protein product | 266882 | 4.45 | 2% |
| 984 | mutant beta-actin (beta′-actin) | 42128 | 5.22 | 20% |
| 984 | unnamed protein product | 55106 | 5.55 | 10% |
| 984 | Aggrecan core protein precursor (Cartilage-specific | 251979 | 4.10 | 1% |
| | proteoglycan core protein) | | | |
| 984 | putative | 2269 | 9.79 | 38% |
| 984 | Chain A, Crystal Structure Of Human Galectin-7 In | 14992 | 7.00 | 17% |
| | Complex With Galactosamine | | | |
| 984 | unnamed protein product | 10988 | 9.19 | 27% |
| 984 | S100 calcium-binding protein A9 | 13291 | 5.71 | 25% |
| 984 | kininogen 1 | 48936 | 6.29 | 2% |
| 984 | cAMP-specific phosphodiesterase PDE4D5 | 84945 | 5.03 | 1% |
| 984 | lumican | 38717 | 6.16 | 5% |
| 984 | creatine kinase M | 43247 | 6.63 | 1% |
| 984 | FLJ00154 protein | 161963 | 5.51 | 2% |
| 984 | kallistatin = tissue kallikrein inhibitor {C-terminal} [human, | 28 | 12.30 | 33% |
| | Peptide Partial, 15 aa] | | | |
| 1018 | Chain A, Antithrombin Iii | 49008 | 5.95 | 58% |
| 1018 | serum vitamin D-binding protein precursor | 53015 | 5.40 | 35% |
| 1018 | angiotensinogen | 53122 | 5.78 | 15% |
| 1018 | complement C4B precursor | 188230 | 7.39 | 7% |
| 1018 | alpha-2-antiplasmin precursor | 54194 | 5.71 | 18% |
| 1018 | kininogen 1 | 47871 | 6.29 | 18% |
| 1018 | blood plasma glutamate carboxypeptidase precursor; | 59893 | 8.02 | 8% |
| | prostate-specific membrane antigen (PSMA) | | | |
| 1018 | chainA, Thyroxine-Binding Globulin complex with | 42452 | 5.69 | 15% |
| | thyroxine | | | |
| 1018 | regucalcin | 33231 | 5.89 | 13% |
| 1018 | alpha 2-plasmin inhibitor, alpha 2-PI {N-terminal, form A} | 2064 | 4.53 | 63% |
| 1018 | lumican | 38375 | 6.16 | 16% |
| 1018 | CXCR4 | 40581 | 8.46 | 2% |
| 1019 | Chain A, antithrombin Iii | 49350 | 5.95 | 66% |
| 1019 | putative | 2269 | 9.79 | 38% |
| 1019 | angiotensinogen | 53407 | 5.78 | 7% |
| 1019 | vitamin D-binding protein precursor | 54526 | 5.40 | 9% |
| 1019 | alpha-2-antiplasmin precursor | 54536 | 5.71 | 4% |
| 1019 | unnamed protein product | 112600 | 8.27 | 0% |
| 1019 | possible protein TPRDII | 205536 | 8.59 | 1% |
| 1019 | PDZ domain protein | 167849 | 4.83 | 2% |
| 1019 | Ral-GDS related protein Rgr | 53054 | 8.17 | 2% |
| 1019 | cardiotropin-like cytokine factor 1 | 25388 | 8.68 | 3% |
| 1021 | putative | 2269 | 9.79 | 38% |
| 1021 | annexin A2 isoform 2 | 38808 | 7.57 | 14% |
| 1021 | Alpha-2-macroglobulin precursor (Alpha-2-M) | 164600 | 6.00 | 2% |
| 1021 | complement component C3 | 188585 | 6.02 | 2% |
| 1021 | hemopexin precursor | 52254 | 6.57 | 8% |
| 1021 | phospholipase D3 isoform 2 | 49196 | 6.00 | 2% |
| 1021 | Beta-2-glycoprotein 1 precursor (Beta-2-glycoprotein I) | 39584 | 8.34 | 2% |
| | (Apolipoprotein H) | | | |
| 1021 | PREDICTED: hypothetical protein LOC137392 isoform 9 | 38661 | 9.94 | 8% |
| 1021 | PREDICTED: similar to ribosomal protein L36 [Pan | 5528 | 10.50 | 21% |
| | troglodytes] | | | |
| 1021 | complement 8 alpha subunit | 66822 | 6.21 | 6% |
| 1021 | kallistatin | 48696 | 7.33 | 2% |
| 1021 | albumin, isoform CRA_a | 25732 | 6.45 | 3% |
| 1021 | hypothetical protein LOC400867 | 15522 | 10.07 | 4% |
| 1021 | cAMP-specific phosphodiesterase PDE4D5 | 84945 | 5.03 | 1% |
| 1055 | Alpha-2-macroglobulin precursor (Alpha-2-M) | 164600 | 6.00 | 4% |
| 1055 | putative | 2269 | 9.79 | 38% |
| 1055 | annexin A2 isoform 2 | 38808 | 7.57 | 11% |
| 1055 | Beta-2-glycoprotein 1 precursor (Beta-2-glycoprotein I) | 39584 | 8.34 | 4% |
| | (Apolipoprotein H) | | | |
| 1055 | dermcidin preproprotein | 11391 | 6.08 | 10% |
| 1055 | kallistatin | 48696 | 7.33 | 4% |
| 1055 | hypothetical protein | 71353 | 5.88 | 2% |
| 1055 | FUSE binding protein 2 | 68735 | 8.52 | 5% |
| 1055 | plasma carboxypeptidase B2 isoform a preproprotein | 48982 | 7.61 | 1% |
| 1055 | PREDICTED: similar to ribosomal protein L36 [Pan | 5528 | 10.50 | 21% |
| | troglodytes] | | | |
| 1055 | cAMP-specific phosphodiesterase PDE4D5 | 84945 | 5.03 | 1% |
| 1055 | cardiotrophin-like cytokine factor 1 | 25388 | 8.68 | 3% |
| 1056 | Alpha 2 macroglobulin variant | 164030 | 6.00 | 21% |
| 1056 | apolipoprotein H precursor | 38287 | 8.34 | 30% |
| 1056 | serine (or cysteine) proteinase inhibitor | 48511 | 7.33 | 21% |
| 1056 | plasma carboxypeptidase B2 isoform a preproprotein | 48411 | 7.61 | 23% |
| 1056 | hypothetical protein | 40328 | 8.67 | 20% |
| 1056 | selenoprotein P | 42758 | 7.87 | 12% |
| 1056 | regucalcin | 33231 | 5.89 | 9% |
| 1056 | neuropolypeptide h3 [human, brain, Peptide, 186 aa] | 20913 | 7.42 | 22% |
| 1056 | phospholipase D3 isoform 2 | 48740 | 6.00 | 2% |
| 1056 | Complement C1r subcomponent precursor | 80122 | 5.89 | 3% |
| 1056 | small proline-rich protein 2D | 7900 | 8.77 | 48% |
| 1056 | dermcidin preproprotein | 11277 | 6.08 | 10% |
| 1056 | hypothetical protein | 69357 | 5.88 | 3% |
| 1056 | FUSE binding protein 2 | 68393 | 8.52 | 6% |
| 1056 | alpha-2-antiplasmin precursor | 54194 | 5.71 | 4% |
| 1056 | PREDICTED: similar to Cyclin G-associated kinase | 146324 | 9.19 | 3% |
| 1056 | hypothetical protein | 130088 | 5.93 | 1% |
| 1063 | putative | 2269 | 9.79 | 38% |
| 1063 | Alpha-2-macroglobulin precursor (Alpha-2-M) | 164600 | 6.00 | 3% |
| 1063 | annexin A2 isoform 2 | 38808 | 7.57 | 6% |
| 1063 | complement component C3 | 188585 | 6.02 | 1% |
| 1063 | dermcidin preproprotein | 11391 | 6.08 | 10% |
| 1063 | Beta-2-glycoprotein 1 precursor (Beta-2-glycoprotein I) | 11391 | 8.34 | 2% |
| | (Apolipoprotein H) | | | |
| 1063 | Complement C1r subcomponent precursor (Complement | 81661 | 5.89 | 1% |
| | component 1, r subcomponent) | | | |
| 1063 | phospholipase D3 isoform 2 | 49196 | 6.00 | 3% |
| 1063 | cAMP-specific phosphodiesterase PDE4D5 | 84945 | 5.03 | 2% |
| 1063 | unnamed protein product | 71246 | 5.92 | 3% |
| 1076 | alpha-2-macroglobulin precursor | 164600 | 6.00 | 18% |
| 1076 | beta-2-glycoprotein 1 precursor | 39584 | 8.34 | 7% |
| 1076 | kallistatin | 48696 | 7.33 | 1% |
| 1089 | alpha-2-macroglobulin precursor | 163175 | 6.00 | 10% |
| 1089 | serine proteinase inhibitor | 48511 | 7.33 | 4% |
| 1089 | beta-2-glycoprotein 1 precursor | 38273 | 8.34 | 18% |
| 1089 | hemopexin precursor | 51512 | 6.57 | 2% |
| 1089 | anti-colorectal carcinoma heavy chain | 50570 | 6.22 | 2% |
| 1094 | apolipoprotein H precursor | 39598 | 8.34 | 33% |
| 1094 | alpha-2-macroglobulin precursor | 164600 | 6.00 | 5% |
| 1094 | cardiotropin-like cytokine factor 1 | 25388 | 8.68 | 3% |
| 1097 | Alpha-2-macroglobulin precursor (Alpha-2-M) | 164600 | 6.00 | 8% |
| 1097 | Beta-2-glycoprotein 1 precursor (Beta-2-glycoprotein I) | 39584 | 8.34 | 5% |
| | (Apolipoprotein H) | | | |
| 1097 | putative | 2269 | 9.79 | 38% |
| 1097 | phospholipase D3 isoform 2 | 49196 | 6.00 | 3% |
| 1097 | DEP domain containing 1, isoform CRA_c | 84786 | 8.71 | 0% |
| 1110 | chain B, crystal structure of fibrinogen | 38081 | 5.84 | 24% |
| 1110 | beta-2-glycoprotein 1 precursor | 39584 | 8.34 | 24% |
| 1110 | alpha 2 macroglobulin | 167505 | 6.06 | 4% |
| 1110 | putative | 2269 | 9.79 | 38% |
| 1110 | dermcidin preproprotein | 11391 | 6.08 | 10% |
| 1110 | annexin A2 isoform 2 | 38808 | 7.57 | 8% |
| 1110 | phospholipase D3 isoform 2 | 49196 | 6.00 | 3% |
| 1110 | hemopexin precursor | 52254 | 6.57 | 4% |
| 1110 | far upstream element (FUSE) binding protein 1, isoform | 69188 | 8.18 | 7% |
| | CRA_c | | | |
| 1110 | Man9-mannosidase | 71004 | 5.92 | 4% |
| 1110 | unnamed protein product | 112600 | 8.27 | 2% |
| 1110 | PREDICTED: similar to ribosomal protein L36 [Pan | 5528 | 10.50 | 21% |
| | troglodytes] | | | |
| 1110 | immunoglobulin lambda light chain VLJ region | 11755 | 8.62 | 9% |
| 1110 | hypothetical protein LOC400867 | 15522 | 10.07 | 4% |
| 1110 | Rho guanine nucleotide exchange factor (GEF) 17 | 223645 | 5.90 | 1% |
| 1110 | cAMP-specific phosphodiesterase PDE4D5 | 84945 | 5.03 | 1% |
| 1125 | Alpha-2-macroglobulin precursor (Alpha-2-M) | 164600 | 6.00 | 9% |
| 1125 | beta-fibrinogen precursor | 55545 | 8.31 | 30% |
| 1125 | peptide, salivary low MW | 1068 | 8.75 | 100% |
| 1125 | kallistatin | 48696 | 7.33 | 6% |
| 1125 | putative | 2269 | 9.79 | 38% |
| 1125 | Beta-2-glycoprotein 1 precursor (Beta-2-glycoprotein I) | 39584 | 8.34 | 6% |
| | (Apolipoprotein H) | | | |
| 1125 | Uncharacterized protein C6orf170 | 14555 | 6.37 | 1% |
| 1125 | histatin 3 | 6145 | 10.09 | 13% |
| 1125 | peptide, salivary low MW | 971 | 8.75 | 100% |
| 1125 | type XVIII collagen | 154430 | 5.45 | 0% |
| 1125 | prolylcarboxypeptidase isoform 1 preproprotein | 56277 | 6.75 | 1% |
| 1125 | Ras-GRF2 | 6046 | 11.71 | 16% |
| 1125 | hypothetical protein LOC400867 | 15522 | 10.07 | 4% |
| 1126 | Chain A, Crystal Structure of Human Beta-2-glycoprotein-I | 37499 | 8.37 | 58% |
| 1126 | alpha-2-macroglobulin precursor | 164600 | 6.00 | 6% |
| 1126 | phospholipase D3 isoform 2 | 49196 | 6.00 | 5% |
| 1126 | myc far upstream element-binding protein | 67664 | 7.21 | 1% |
| 1126 | coronin-like protein | 51722 | 6.12 | 3% |
| 1126 | Chain B, Crystal Structure of Fibrinogen | 38081 | 5.84 | 6% |
| 1128 | alpha-2-macroglobulin precursor | 164600 | 6.00 | 3% |
| 1128 | prolylcarboxypeptidase | 56277 | 6.75 | 3% |
| 1128 | hemopexin precursor | 52254 | 6.57 | 8% |
| 1128 | beta-2-glycoprotein | 39584 | 8.34 | 2% |
| 1128 | complement C1r subcomponent precursor | 81661 | 5.89 | 7% |
| 1128 | serine dehydrase, isoform CRA_b | 45379 | 9.14 | 7% |
| 1139 | beta-2-glycoprotein 1 precursor | 38273 | 8.34 | 23% |
| 1139 | alpha-1-B-glycoprotein | 51908 | 5.65 | 18% |
| 1139 | chain B, structure of complement C3b | 103886 | 5.18 | 16% |
| 1139 | fibrinogen beta chain, isoform CRA_g | 50370 | 8.63 | 8% |
| 1139 | filaggrin 2 | 247928 | 8.45 | 1% |
| 1139 | alpha-amylase | 33074 | 7.56 | 7% |
| 1139 | hypothetical protein | 40328 | 8.67 | 7% |
| 1139 | aldehyde dehydrogenase I | 36718 | 7.12 | 5% |
| 1139 | Alpha-2-macroglobulin precursor (Alpha-2-M) | 163175 | 6.00 | 4% |
| 1139 | PREDICTED: similar to membrane-associated ring finger | 78153 | 9.27 | 4% |
| | (C3HC4) 4 | | | |
| 1139 | arginase (EC 3.5.3.1) | 34712 | 7.10 | 6% |
| 1140 | Alpha-2-macroglobulin precursor (Alpha-2-M) | 164600 | 6.00 | 9% |
| 1140 | kallistatin | 48696 | 7.33 | 27% |
| 1140 | beta-2-glycoprotein 1 precursor | 39584 | 8.34 | 17% |
| 1140 | beta-fibrinogen precursor | 55545 | 8.31 | 10% |
| 1140 | trypsin inhibitor | 107103 | 6.58 | 5% |
| 1140 | putative | 2269 | 9.79 | 38% |
| 1140 | annexin A2 isoform 2 | 38808 | 7.57 | 10% |
| 1140 | phospholipase D3 isoform 2 | 49196 | 6.00 | 5% |
| 1140 | dermcidin preproprotein | 11391 | 6.08 | 10% |
| 1140 | alpha-fibrinogen precursor | 70223 | 8.26 | 3% |
| 1140 | prolylcarboxypeptidase isoform 1 preproprotein | 56277 | 6.75 | 3% |
| 1140 | Plasminogen | 93263 | 6.89 | 2% |
| 1140 | unnamed protein product | 112600 | 8.27 | 0% |
| 1140 | unnamed protein product | 33844 | 6.67 | 3% |
| 1140 | cAMP-specific phosphodiesterase PDE4D5 | 84945 | 5.03 | 1% |
| 1140 | PREDICTED: similar to ribosomal protein L36 [Pan | 5528 | 10.50 | 21% |
| | troglodytes] | | | |
| 1140 | small proline-rich protein | 8676 | 9.07 | 25% |
| 1140 | Rho guanine nucleotide exchange factor (GEF) 17 | 223645 | 5.90 | 1% |
| 1146 | beta-fibrinogen precursor | 55545 | 8.31 | 51% |
| 1146 | apolipoprotein H precursor | 39598 | 8.34 | 31% |
| 1146 | phospholipase D3 isoform 2 | 49196 | 6.00 | 8% |
| 1146 | putative | 2269 | 9.79 | 38% |
| 1146 | plasma serine protease inhibitor precursor | 45886 | 9.38 | 2% |
| 1146 | alpha-2-macroglobulin precursor | 164600 | 6.00 | 4% |
| 1146 | unnamed protein product | 112600 | 8.27 | 0% |
| 1146 | golgi membrane protein GP73 | 45346 | 4.91 | 1% |
| 1147 | alpha 2 macroglobulin | 167505 | 6.06 | 18% |
| 1147 | apolipoprotein H precursor | 39598 | 8.34 | 22% |
| 1147 | fibrinogen beta chain, isoform CRA-d | 52749 | 8.33 | 45% |
| 1147 | phospholipase D3 isoform 2 | 49196 | 6.00 | 6% |
| 1147 | annexin A2 | 32600 | 5.93 | 5% |
| 1147 | PRSS3 | 27040 | 6.89 | 8% |
| 1147 | plasminogen | 93263 | 6.89 | 2% |
| 1147 | ankyrin-3 | 482387 | 6.12 | 0% |
| 1147 | cAMP-specific phosphodiesterase 4D | 23995 | 9.75 | 15% |
| 1147 | solute carrierfamily 4, sodium bicarbonate cotransporter, | 110494 | 8.62 | 0% |
| | member 5, isoform CRA_b | | | |
| 1156 | fibrinogen beta chain | 52759 | 8.33 | 57% |
| 1156 | apolipoprotein H precursor | 39598 | 8.34 | 37% |
| 1156 | annexin A2 | 38808 | 7.57 | 24% |
| 1156 | fibrinogen alphaA | 49708 | 5.48 | 8% |
| 1156 | alpha-figrinogen precursor | 70223 | 8.26 | 10% |
| 1156 | adenylyl cyclase-associated protein | 51926 | 8.07 | 13% |
| 1156 | phospholipase D3 isoform 2 | 49196 | 6.00 | 2% |
| 1156 | trypsinogen IV b-form | 28611 | 5.86 | 8% |
| 1156 | myc far upstream element-binding protein | 67664 | 7.21 | 3% |
| 1156 | plasminogen | 93263 | 6.89 | 5% |
| 1156 | solute carrier family 4 | 110494 | 8.62 | 0% |
| 1156 | chain b, human complement component C3 | 114238 | 5.55 | 3% |
| 1156 | Rho guanine nucleotide exchange factor 17 | 223645 | 5.90 | 0% |
| 1158 | fibrinogen beta chain, isoform CRA_d | 52131 | 8.33 | 63% |
| 1158 | beta-2-glycoprotein 1 precursor | 38273 | 8.34 | 19% |
| 1158 | annexin A2 isoform 2 | 38580 | 7.57 | 21% |
| 1158 | fibrinogen alphaA | 49366 | 5.48 | 6% |
| 1158 | phospholipase D3 isoform 2 | 48740 | 6.00 | 2% |
| 1158 | plasma serine protease inhibitor precursor | 45772 | 9.38 | 2% |
| 1158 | Plasminogen | 90526 | 6.89 | 2% |
| 1158 | B-cell translocation gene 2 | 17405 | 8.29 | 4% |
| 1158 | unnamed protein product | 55188 | 9.10 | 4% |
| 1169 | fibrin beta | 51358 | 7.95 | 61% |
| 1169 | fibrinogen beta chain, isoform CRA_b | 28040 | 8.16 | 34% |
| 1169 | fibrinogen alphaA | 49708 | 5.48 | 8% |
| 1169 | annexin A2 isoform 2 | 38808 | 7.57 | 14% |
| 1169 | alpha-fibrinogen precursor | 70223 | 8.26 | 9% |
| 1169 | putative | 2269 | 9.79 | 38% |
| 1169 | plasma serine protease inhibitor precursor | 45886 | 9.38 | 5% |
| 1169 | dermcidin preproprotein | 11391 | 6.08 | 10% |
| 1169 | phospholipase D3 isoform 2 | 49196 | 6.00 | 2% |
| 1169 | adenylyl cyclase-associated protein | 51926 | 8.07 | 2% |
| 1169 | Beta-2-glycoprotein 1 precursor (Beta-2-glycoprotein I) | 39584 | 8.34 | 4% |
| | (Apolipoprotein H) | | | |
| 1169 | beta-fibrinogen | 7107 | 10.12 | 13% |
| 1169 | Plasminogen | 93263 | 6.89 | 2% |
| 1169 | acid sphingomyelinase-like phosphodiesterase | 20848 | 6.33 | 3% |
| 1169 | IGHG1 protein | 52367 | 7.88 | 4% |
| 1169 | cAMP-specific phosphodiesterase PDE4D5 | 84945 | 5.03 | 7% |
| 1174 | serum vitamin D-binding protein precursor | 54612 | 5.40 | 64% |
| 1174 | complement component C4A | 194337 | 6.65 | 2% |
| 1174 | alpha-2-antiplasmin precursor | 54536 | 5.71 | 5% |
| 1174 | putative | 2269 | 9.79 | 38% |
| 1174 | serpin B8 | 43328 | 5.43 | 4% |
| 1174 | antithrombin III | 53041 | 6.32 | 2% |
| 1174 | alpha 2-plasmin inhibitor | 2064 | 4.53 | 63% |
| 1174 | lumican | 38717 | 6.16 | 2% |
| 1174 | unnamed protein product | 112600 | 8.27 | 0% |
| 1174 | unnamed protein product | 16391 | 6.21 | 5% |
| 1187 | chain B, structure of complement C3b | 104912 | 5.18 | 30% |
| 1187 | alpha-1-antitrypsin | 46848 | 5.43 | 32% |
| 1187 | chain I, P14-fluorescein-N135q-S380c-antithrombin-Iii | 49388 | 5.95 | 39% |
| 1187 | hemopexin precursor | 52254 | 6.57 | 32% |
| 1187 | alpha-1-B-glycoprotein | 52479 | 5.65 | 16% |
| 1187 | ceruloplasmin | 98321 | 5.29 | 12% |
| 1187 | phospholipid transfer protein | 44989 | 8.69 | 20% |
| 1187 | putative | 2269 | 9.97 | 38% |
| 1187 | afamin precursor | 70963 | 5.64 | 3% |
| 1187 | unnamed protein product | 70723 | 5.53 | 9% |
| 1187 | proapolipoprotein | 28944 | 5.45 | 18% |
| 1187 | inter-alpha-trypsin inhibitor C-terminal | 93745 | 6.02 | 3% |
| 1187 | histidine-rich glycoprotein precursor | 60510 | 7.09 | 8% |
| 1187 | vitamin D-binding protein precursor | 54526 | 5.40 | 8% |
| 1187 | dermcidin preproprotein | 11391 | 6.08 | 10% |
| 1187 | complement C4B precursor | 189599 | 7.39 | 3% |
| 1187 | inter-alpha-trypsin inhibitor family heavy chain-related | 103536 | 6.64 | 2% |
| | protein | | | |
| 1187 | Plasminogen | 93263 | 6.89 | 1% |
| 1187 | alpha-2-antiplasmin precursor | 54536 | 5.71 | 2% |
| 1187 | Chain A, Crystal Structure Of Native Heparin Cofactor Ii | 55096 | 6.26 | 3% |
| 1187 | cAMP-specific phosphodiesterase PDE4D5 | 84945 | 5.03 | 1% |
| 1187 | complement 9 | 661728 | 5.42 | 7% |
| 1187 | Alpha-2-macroglobulin precursor (Alpha-2-M) | 164600 | 6.00 | 1% |
| 1187 | Vitamin K-dependent protein S precursor | 77127 | 5.48 | 2% |
| 1187 | lumican | 38717 | 6.16 | 4% |
| 1187 | hyaluronan binding protein 2 | 64740 | 6.09 | 1% |
| 1187 | Chain A, Crystallographic Analysis Of The Human Vitamin | 52780 | 5.17 | 54% |
| | D Binding Protein | | | |
| 1187 | antithrombin III | 53041 | 6.32 | 23% |
| 1187 | alpha-2-antiplasmin precursor | 54536 | 5.71 | 16% |
| 1187 | putative | 2269 | 9.79 | 38% |
| 1187 | complement component C4A | 194337 | 6.65 | 1% |
| 1187 | alpha 2-plasmin inhibitor, alpha 2-PI {N-terminal, form A} | 2064 | 5.43 | 63% |
| | [human, plasma, Peptide Partial, 19 aa] | | | |
| 1187 | lumican | 38717 | 6.16 | 15% |
| 1187 | dermcidin preproprotein | 11391 | 6.08 | 10% |
| 1187 | ectonucleotide pyrophosphatase/phosphodiesterase 5 | 54745 | 5.94 | 2% |
| | (putative function) | | | |
| 1187 | kininogen 1 | 48936 | 6.29 | 2% |
| 1187 | Plasminogen | 93263 | 6.89 | 1% |
| 1187 | alpha2-HS glycoprotein | 36268 | 5.20 | 2% |
| 1187 | fetuin-like protein IRL685 | 42922 | 6.87 | 3% |
| 1187 | Creatine kinase, muscle | 43268 | 6.77 | 7% |
| 1187 | PREDICTED: similar to Cyclin G-associated kinase | 148776 | 9.19 | 0% |
| 1187 | KIAA1277 protein | 118159 | 9.14 | 1% |
| 1206 | trypsin inhibitor | 106647 | 6.58 | 6% |
| 1206 | Chain A, Annexin A2: Does It Induce Membrane | 38638 | 6.92 | 9% |
| | Aggregation By A New Multimeric State Of The Protein | | | |
| 1206 | prepro-C3b/C4B inactivator | 65725 | 7.72 | 10% |
| 1206 | trypsin (EC 3.4.21.4) III precursor - human | 26759 | 5.95 | 15% |
| 1206 | hemopexin precursor | 51512 | 6.57 | 13% |
| 1206 | phospholipase D3 isoform 2 | 48740 | 6.00 | 2% |
| 1206 | plasma serine protease inhibitor precursor | 45772 | 9.38 | 2% |
| 1206 | WD repeat domain, phosphoinositide interacting 2 isoform b | 47513 | 5.68 | 2% |
| 1214 | prepro-C3b/C4B inactivator | 63725 | 7.72 | 26% |
| 1214 | complement component C3 | 187046 | 6.02 | 6% |
| 1214 | pigment epithelial-differentiating factor | 46300 | 5.84 | 13% |
| 1214 | immunoglobulin heavy chain variable region | 9196 | 6.92 | 13% |
| 1214 | lymphoid-restricted membrane protein | 56178 | 5.44 | 1% |
| 1214 | neuron growth inhibitory factor | 6932 | 4.79 | 22% |
| 1215 | unnamed protein product | 65696 | 7.38 | 27% |
| 1215 | glucosamine | 62840 | 8.60 | 6% |
| 1215 | fibrinogen beta chain | 52759 | 8.33 | 12% |
| 1215 | hemopexin precursor | 52254 | 6.57 | 4% |
| 1215 | alpha-2-antiplasmin precursor | 54536 | 5.71 | 2% |
| 1215 | phospholipase D3 | 49196 | 6.00 | 2% |
| 1215 | complement component C3 | 188585 | 6.02 | 0% |
| 1215 | cardiotrophin-like cytokine factor 1 | 25388 | 8.68 | 3% |
| 1238 | hemopexin precursor | 51512 | 6.57 | 32% |
| 1238 | beta-fibrinogen precursor | 54861 | 8.31 | 32% |
| 1238 | prepro-C3b/C4B inactivator | 65725 | 7.72 | 6% |
| 1238 | dermcidin preproprotein | 11277 | 6.08 | 20% |
| 1238 | Fc fragment of IgG binding protein | 571718 | 5.14 | 1% |
| 1238 | phospholipase D3 isoform 2 | 48740 | 6.00 | 2% |
| 1238 | annexin A2 isoform 2 | 38580 | 7.57 | 6% |
| 1238 | unnamed protein product | 15956 | 7.12 | 8% |
| 1238 | Inter-alpha-trypsin inhibitor heavy chain H1 precursor (ITI | 101326 | 6.31 | 4% |
| | heavy chain H1) (Inter-alpha-inhibitor heavy chain 1) | | | |
| | (Inter-alpha-trypsin inhibitor complex component III) | | | |
| | (Serum-derived hyaluronan-associated protein) (SHAP) | | | |
| 1238 | Beta-2-glycoprotein 1 precursor (Beta-2-glycoprotein I) | 38273 | 8.34 | 2% |
| | (Apolipoprotein H) (Apo-H) (B2GPI) (Beta(2)GPI) | | | |
| | (Activated protein C-binding protein) (APC inhibitor) | | | |
| | (Anticardiolipin cofactor) | | | |
| 1238 | regucalcin | 33231 | 5.89 | 5% |
| 1238 | granulocyte colony-stimulating factor receptor | 85066 | 8.33 | 0% |
| 1238 | cAMP-specific phosphodiesterase PDE4D5 | 84375 | 5.03 | 1% |
| 1242 | fibrinogen beta chain, isoform CRA_d | 52759 | 8.33 | 25% |
| 1242 | putative | 2269 | 9.79 | 38% |
| 1242 | annexin A2 isoform 2 | 38808 | 7.57 | 6% |
| 1242 | hemopexin precursor | 52254 | 6.57 | 4% |
| 1242 | phospholipase D3 isoform 2 | 49196 | 6.00 | 2% |
| 1242 | antithrombin III | 53041 | 6.32 | 3% |
| 1242 | beta globin chain | 11537 | 5.90 | 21% |
| 1242 | granulocyte colony-stimulating factor receptor | 86548 | 8.33 | 0% |
| 1242 | KIAA1904 protein | 90787 | 7.63 | 1% |
| 1242 | complement component C3 | 188585 | 6.02 | 0% |
| 1242 | unnamed protein product | 71246 | 5.92 | 1% |
| 1242 | aberrant LSLCL | 33536 | 6.82 | 6% |
| 1263 | hemopexin precursor | 52254 | 6.57 | 17% |
| 1263 | prepro-C3b/C4B inactivator | 68120 | 7.72 | 9% |
| 1263 | beta-fibrinogen precursor | 55545 | 8.31 | 10% |
| 1263 | phospholipase D3 isoform 2 | 49196 | 6.00 | 2% |
| 1263 | annexin A2 | 38808 | 7.57 | 6% |
| 1263 | unnamed protein product | 16070 | 7.12 | 14% |
| 1263 | regucalcin | 33802 | 5.89 | 5% |
| 1263 | trypsin | 27329 | 5.95 | 5% |
| 1263 | lysosomal acid phosphatase 2 precursor | 48713 | 6.28 | 4% |
| 1263 | cAMP-specific phosphodiesterase PDE4D5 | 84945 | 5.03 | 1% |
| 1263 | granulocyte colony-stimulating factor receptor | 86548 | 8.33 | 0% |
| 1263 | chain A, Hr1b Domain Form Prk1 | 9054 | 9.77 | 11% |
| 1290 | hemopexin precursor | 52254 | 6.57 | 14% |
| 1290 | annexin A2 isoform 2 | 38808 | 7.57 | 6% |
| 1290 | glucosamine (N-acetyl)-6-sulfatase precursor | 62840 | 8.60 | 3% |
| 1290 | regucalcin | 33802 | 5.89 | 5% |
| 1290 | unnamed protein product | 16070 | 7.12 | 8% |
| 1290 | phospholipase D3 isoform 2 | 49196 | 6.00 | 2% |
| 1290 | Plasminogen | 93263 | 6.89 | 1% |
| 1290 | cAMP-specific phosphodiesterase PDE4D5 | 84945 | 5.03 | 1% |
| 1290 | granulocyte colony-stimulating factor receptor | 86548 | 8.33 | 5% |
| 1290 | hCG2007940 | 12771 | 10.89 | 6% |
| 1290 | Rho guanine nucleotide exchange factor (GEF) 17 | 223645 | 5.90 | 0% |
| 1323 | annexin A2 isoform 2 | 38580 | 7.57 | 12% |
| 1323 | glucosamine (N-acetyl)-6-sulfatase precursor | 62042 | 8.60 | 7% |
| 1323 | hemopexin, isoform CRA_d | 43201 | 6.24 | 9% |
| 1323 | plasma serine protease inhibitor precursor | 45772 | 9.38 | 5% |
| 1323 | Trypsin-3 precursor (Trypsin III) (Brain trypsinogen) | 32478 | 7.46 | 10% |
| | (Mesotrypsinogen) | | | |
| 1323 | cartilage linking protein 1 | 40140 | 7.10 | 2% |
| 1323 | dermcidin preproprotein | 11277 | 6.08 | 10% |
| 1323 | phospholipase D3 isoform 2 | 48740 | 6.00 | 2% |
| 1323 | glutathione S-transferase GSTM5-5 | 25676 | 6.90 | 11% |
| 1326 | procollagen C-endopeptidase enhancer 1 precursor | 48797 | 7.41 | 47% |
| 1326 | homopexin precursor | 52254 | 6.57 | 36% |
| 1326 | cartilage linking protein 1 | 40767 | 7.10 | 22% |
| 1326 | dynein light chain 2 | 10457 | 6.81 | 26% |
| 1326 | trypsin (EC 3.4.21.4) III precursor - human | 27329 | 5.95 | 9% |
| 1326 | Chain A, Structure Of Human Annexin A2 In The Presence | 36631 | 8.32 | 21% |
| | Of Calcium Ions | | | |
| 1326 | Chain A, Structure Of Human Trypsin Iv (Brain Trypsin) | 24832 | 6.56 | 10% |
| 1326 | lysosomal acid phosphatase 2 precursor | 48713 | 6.28 | 8% |
| 1326 | alpha-2-macroglobulin precursor | 164600 | 6.00 | 5% |
| 1326 | PREDICTED: similar to Hornerin | 188565 | 9.82 | 5% |
| 1326 | type II intermediate filament of hair keratin | 54756 | 5.48 | 11% |
| 1326 | filaggrin 2 | 249296 | 8.45 | 0% |
| 1326 | Beta-2-glycoprotein 1 precursor (Beta-2-glycoprotein I) | 39584 | 8.34 | 2% |
| | (Apolipoprotein H) | | | |
| 1326 | complement component C3 | 188585 | 6.02 | 3% |
| 1326 | glutathione transferase M3 | 27127 | 5.37 | 4% |
| 1326 | glucosamine (N-acetyl)-6-sulfatase precursor | 62840 | 8.60 | 2% |
| 1326 | phospholipase D3 isoform 1 | 55127 | 6.02 | 6% |
| 1326 | regucalcin | 33802 | 5.89 | 5% |
| 1326 | Chain B, Non-Covalent Complex Between Alpha-1-Pi- | 26077 | 8.23 | 4% |
| | Pittsburgh And S195a Trypsin | | | |
| 1326 | general transcription factor IIIC, polypeptide 4, 90 kDa | 93140 | 6.27 | 1% |
| 1326 | dermcidin preproprotein | 11391 | 6.08 | 10% |
| 1327 | hemopexin precursor | 52254 | 6.75 | 41% |
| 1327 | enolase 1 | 47481 | 7.10 | 35% |
| 1327 | coronin-like protein | 51722 | 6.12 | 11% |
| 1327 | alpha-2-macroglobulin precursor | 164600 | 6.00 | 7% |
| 1327 | annexin A2 isoform 2 | 38808 | 7.57 | 12% |
| 1327 | complement component C3 | 188585 | 6.02 | 3% |
| 1327 | glucosamine (N-acetyl)-6-sulfatase precursor | 62840 | 8.60 | 2% |
| 1327 | trypsin III precursor | 27329 | 5.95 | 9% |
| 1327 | arylsulfatase B precursor | 60195 | 8.30 | 3% |
| 1327 | phospholipase D3 isoform 2 | 49196 | 6.00 | 4% |
| 1327 | lysosomal acid phosphatase 2 precursor | 48713 | 6.28 | 5% |
| 1327 | PREDICTED: similar to ribosomal protein L36 | 5528 | 10.50 | 21% |
| 1327 | antigen MLAA-44 | 42961 | 5.11 | 1% |
| 1327 | granulocyte colony-stimulating factor receptor | 86548 | 8.33 | 0% |
| 1327 | angiotensin I converting enzyme 1 | 134639 | 5.98 | 1% |
| 1361 | complement component C3 | 188585 | 6.02 | 11% |
| 1361 | annexin A2 isoform 2 | 38808 | 7.57 | 16% |
| 1361 | lysosomal acid phosphatase 2 precursor | 48713 | 6.28 | 8% |
| 1361 | protease serine 1 | 9218 | 10.28 | 30% |
| 1361 | carboxypeptidase N, polypetide 1, 50 kD precursor | 52538 | 6.86 | 6% |
| 1361 | cartilage linking protein 1 | 40767 | 7.10 | 11% |
| 1361 | phospholipase D3 isoform 2 | 49196 | 6.00 | 2% |
| 1361 | dermcidin preproprotein | 11391 | 6.08 | 10% |
| 1361 | neuropolypeptide h3 | 16068 | 8.81 | 14% |
| 1361 | arylsulfatase B precursor | 60195 | 8.43 | 8% |
| 1361 | Plasminogen | 93263 | 6.89 | 1% |
| 1361 | chain, annexin I | 35246 | 7.77 | 5% |
| 1361 | hemopexin precursor | 52254 | 6.57 | 6% |
| 1361 | mutant beta-globin | 12635 | 5.09 | 20% |
| 1361 | alpha-fibrinogen precursor | 70223 | 8.26 | 5% |
| 1361 | unnamed protein product | 79911 | 8.51 | 1% |
| 1548 | afamin precursor | 69024 | 5.64 | 10% |
| 1548 | trypsin inhibitor | 106647 | 6.58 | 1% |
| 1548 | Inter-alpha-trypsin inhibitor heavy chain H1 precursor (ITI | 101326 | 6.31 | 5% |
| | heavy chain H1) (Inter-alpha-inhibitor heavy chain 1) | | | |
| | (Inter-alpha-trypsin inhibitor complex component III) | | | |
| | (Serum-derived hyaluronan-associated protein) (SHAP) | | | |
| 1548 | Alpha-2-macroglobulin precursor (Alpha-2-M) | 163175 | 6.00 | 1% |
| 1548 | beta-fibrinogen precursor | 54861 | 8.31 | 11% |
| 1548 | anti-colorectal carcinoma heavy chain | 50570 | 6.22 | 2% |
| 1646 | unnamed protein product | 70723 | 5.53 | 15% |
| 1646 | annexin A2 isoform 2 | 38808 | 7.57 | 15% |
| 1646 | transaldolase 1 | 37688 | 6.36 | 14% |
| 1646 | putative | 2269 | 9.97 | 38% |
| 1646 | dermcidin preproprotein | 11391 | 6.08 | 10% |
| 1646 | tousled-like kinase 1 | 82461 | 8.82 | 2% |
| 1778 | apolipoprotein J precursor | 49342 | 6.27 | 22% |
| 1778 | chain A, annexin V | 35840 | 4.94 | 8% |
| 1778 | annexin A2 isoform 2 | 38808 | 7.57 | 10% |
| 1778 | inter-alpha-trypsin inhibitor | 103549 | 6.51 | 3% |
| 1778 | trypsin-3 precursor | 33276 | 7.46 | 10% |
| 1778 | complement factor B | 86819 | 6.55 | 1% |
| 1778 | apolipoprotein E | 36302 | 5.65 | 6% |
| 1778 | regucalcin | 33802 | 5.89 | 5% |
| 1778 | filaggrin 2 | 249296 | 8.45 | 1% |
| 1778 | chain, x-ray crystal structure of C3d | 32959 | 6.34 | 9% |
| 1778 | plasminogen | 93263 | 6.89 | 1% |
| 1778 | cAMP-specific phosphodiesterase PDE4D5 | 84945 | 5.03 | 2% |
| 2045 | complement component C3 | 188585 | 6.02 | 7% |
| 2045 | poly-Ig | 76443 | 5.38 | 4% |
| 2045 | putative | 2269 | 9.79 | 38% |
| 2045 | PREDICTED: similar to CG6405-PA | 37748 | 6.37 | 2% |
| 2045 | Chain A, Crystal Structure Of Human Tr Alpha Bound T3 | 30656 | 5.41 | 14% |
| | In Orthorhombic Space Group | | | |
| 2045 | LARP | 42148 | 5.48 | 4% |
| 2045 | hypothetical protein LOC400867 | 15522 | 10.07 | 4% |
| 2158 | putative | 2269 | 9.79 | 38% |
| 2284 | chain A . . . apolipoprotein | 28061 | 5.27 | 62% |
| 2284 | putative | 2269 | 9.79 | 38% |
| 2284 | tenascin | 246210 | 4.80 | 0% |
| 2284 | Chain A, Structure Of Human Trypsin Iv (Brain Trypsin) | 24832 | 6.56 | 5% |
| 2284 | glutathione transferase M3 | 27127 | 5.37 | 6% |
| 2284 | chain A, aspartylglucosaminidase | 17552 | 4.82 | 9% |
| 2284 | unnamed protein product | 23195 | 5.48 | 5% |
| 2284 | dermcidin preproprotein | 11391 | 6.08 | 10% |
| 2284 | ATP binding domain 3 | 37296 | 9.58 | 2% |
| 2284 | cAMP-specific phosphodiesterase PDE4D5 | 84945 | 5.03 | 5% |
| 2285 | Chain A, Crystal Structure of Lipid-Free human | 28061 | 5.27 | 82% |
| | apolipoproprotein A-I | | | |
| 2285 | unnamed protein | 23195 | 5.48 | 24% |
| 2285 | putative | 2269 | 9.79 | 38% |
| 2285 | DEP domain containing 1, isoform CRA | 84786 | 8.71 | 0% |
| 2285 | glutathione transferase M3 | 27127 | 5.37 | 7% |
| 2285 | PREDICTED: hypothetical protein | 47324 | 12.09 | 1% |
| 2285 | unnamed protein product | 71246 | 5.92 | 1% |
| 2285 | Chain A, Trypsin (E.C.3.4.21.4) Complexed With The | 24669 | 7.64 | 3% |
| | Inhibitor Diisopropyl-Fluorophosphofluoridate (Dfp) | | | |
| 2305 | Chain A, Crystal Structure of Lipid-Free Human | 28061 | 5.27 | 44% |
| | Apolipoprotein A-1 | | | |
| 2305 | putative | 2269 | 9.79 | 38% |
| 2305 | dermcidin preproprotein | 11391 | 6.08 | 10% |
| 2305 | plasminogen | 93263 | 6.89 | 1% |
| 2305 | hCG22067 | 30319 | 7.98 | 3% |
| 2305 | unnamed protein product | 71246 | 5.92 | 1% |
| 2305 | supported by mouse EST AA538043 (NID: g2284036) | 37423 | 9.28 | 2% |
| 2305 | cAMP-specific phosphodiesterase PDE4D5 | 84945 | 5.03 | 2% |
| 2305 | Chain A, Human Aspartylglucosaminidase | 17552 | 4.82 | 4% |
| 2364 | C-type lectin domain family 3, member b, isoform CRA_a | 22921 | 5.52 | 37% |
| 2364 | hypothetical protein LOC79017 | 21222 | 5.07 | 8% |
| 2364 | putative | 2269 | 9.79 | 38% |
| 2364 | lipoprotein Gln I | 28329 | 5.27 | 26% |
| 2364 | unnamed protein product | 112600 | 8.27 | 0% |
| 2364 | PRSS3 protein | 27040 | 6.86 | 8% |
| 2364 | Chain A, Solution Structure Of The Human Defensin Hbd-2 | 4345 | 9.50 | 16% |
| 2364 | KIAA1481 protein | 150746 | 6.77 | 0% |
| 2609 | putative | 2269 | 9.79 | 38% |
| 2609 | Chain B, Crystal Structure Of A Rac-Rhogdi Complex | 20521 | 6.16 | 12% |
| 2609 | dismutase, Cu/Zn superoxide | 16020 | 8.76 | 16% |
| 2609 | hypothetical protein LOC51250 | 28038 | 9.31 | 2% |
| 2609 | proteasome 26S non-ATPase subunit 10 isoform 1 | 24697 | 5.71 | 6% |
| 2609 | hCG2044987 | 11472 | 9.73 | 8% |
| 44 | fibronectin precursor | 266034 | 5.45 | 27% |
| 44 | complement component C3 | 188585 | 6.02 | 17% |
| 44 | alpha-2-macroglobulin precursor | 164600 | 6 | 10% |
| 44 | hemopexin precursor | 52254 | 6.57 | 27% |
| 44 | semenogelin II precursor | 65519 | 9.08 | 3% |
| 44 | cAMP-specific phosphodiesterase | 84945 | 5.03 | 2% |
| 82 | fibronectin precursor | 266034 | 5.45 | 7% |
| 82 | plasma protease (C1) inhibitor precursor | 55375 | 6.09 | 16% |
| 82 | Chain B, Structure of Complement C3b | 104912 | 5.18 | 8% |
| 82 | megakaryocyte stimulating factor | 152195 | 9.53 | 4% |
| 82 | alpha-1-antitrypsin | 46848 | 5.43 | 14% |
| 109 | fibronectin 1 isoform 3 preproprotein | 262656 | 5.49 | 23% |
| 109 | factor H | 143710 | 6.28 | 7% |
| 109 | alpha-1-antitrypsin | 13859 | 5.43 | 21% |
| 109 | alpha-2-macroglobulin precursor | 164600 | 6 | 1% |
| 109 | complement component C3 | 188585 | 6.02 | 2% |
| 126 | fibronectin 1 isoform 3 preproprotein | 262656 | 5.49 | 22% |
| 126 | alpha-2-macroglobulin precursor | 164600 | 6 | 10% |
| 126 | complement component C3 | 188585 | 6.02 | 3% |
| 126 | unnamed protein, serum protein | 71246 | 5.92 | 6% |
| 126 | gelsolin | 86043 | 5.9 | 4% |
| 126 | hemopexin precursor | 52254 | 6.57 | 2% |
| 164 | fibronectin 1 isoform 3 preproprotein | 262656 | 5.49 | 23% |
| 164 | macroglobulin alpha 2 | 162072 | 5.95 | 19% |
| 164 | gelsolin | 52511 | 5.21 | 6% |
| 175 | fibronectin precursor | 260064 | 5.45 | 6% |
| 175 | inter-alpha-trypsin family heavy chain-related protein | 103549 | 6.51 | 6% |
| 175 | thyroxine-binding globulin precursor | 46637 | 5.87 | 11% |
| 175 | afamin precursor | 70963 | 5.64 | 1% |
| 175 | complement component C3 | 188585 | 6.02 | 4% |
| 175 | attractin-2 | 146159 | 6.65 | 1% |
| 175 | annexin A2 isoform 2 | 38808 | 7.57 | 7% |
| 175 | alpha-1-antitrypsin | 13859 | 7.93 | 21% |
| 184 | alpha 2 macroglobulin | 167505 | 6.06 | 21% |
| 184 | mutant beta-globin | 16098 | 7.14 | 19% |
| 191 | alpha-1-antitrypsin precursor | 46849 | 5.51 | 22% |
| 191 | alpha-2-macroglobulin precursor | 164,000 | 6 | 8% |
| 191 | complement component C3 | 188585 | 6.02 | 5% |
| 191 | hemopexin precursor | 52254 | 6.57 | 9% |
| 191 | fibronectin precursor | 260064 | 5.45 | 3% |
| 205 | alpha-1-antichymotrypsin | 48834 | 5.79 | 3% |
| 205 | aggrecan core protein precursor | 251979 | 4.1 | 0% |
| 205 | creatine kinase M | 43247 | 6.63 | 3% |
| 213 | alpha-1-antitrypsin | 22871 | 6.11 | 36% |
| 216 | collagen type IV alpha 1 | 161654 | 8.55 | 3% |
| 216 | annexin A2 isoform 2 | 38808 | 7.57 | 16% |
| 216 | alpha 2 type IV collagen preproprotein | 168646 | 8.89 | 5% |
| 244 | collagen type IV alpha 1 | 161654 | 8.55 | 3% |
| 244 | annexin A2 isoform 2 | 38808 | 7.57 | 16% |
| 244 | alpha 2 type IV collagen preproprotein | 168646 | 8.89 | 5% |
| 252 | alpha 2 macroglobulin | 167505 | 6.06 | 23% |
| 252 | complement factor H | 143694 | 6.21 | 24% |
| 252 | complement component C3 | 188585 | 6.02 | 9% |
| 252 | inter-alpha-trypsin inhibitor C-terminal | 93745 | 6.02 | 2% |
| 252 | hemopexin precursor | 52254 | 6.57 | 8% |
| 252 | annexin A2 isoform 2 | 38808 | 7.57 | 5% |
| 267 | Human Factor H (Four models determined by solution | 141553 | 6.19 | 10% |
| | Scattering) | | | |
| 267 | inter-alpha-trypsin inhibitor | 103549 | 6.51 | 8% |
| 267 | fibronectin precursor | 260064 | 5.45 | 2% |
| 267 | afamin precursor | 70963 | 5.64 | 5% |
| 267 | complement C4B precursor | 189599 | 7.39 | 8% |
| 267 | serpin peptidase inhibitor, clade B | 44313 | 5.32 | 15% |
| 267 | alpha-2-macroglobulin | 164600 | 6 | 1% |
| 267 | inter-alpha-trypsin inhibitor, C-terminal | 93745 | 6.02 | 1% |
| 267 | C9 complement protein | 64399 | 5.49 | 1% |
| 267 | ceruloplasmin | 98321 | 5.29 | 2% |
| 267 | serpin B8 | 43328 | 5.43 | 10% |
| 295 | complement factor H | 143694 | 6.21 | 34% |
| 295 | alpha 2 macroblobulin | 167505 | 6.06 | 15% |
| 295 | trypsin inhibitor | 107103 | 6.58 | 10% |
| 295 | inter-alpha-trypsin inhibitor | 101782 | 6.31 | 5% |
| 295 | complement component C3 | 188585 | 6.02 | 2% |
| 305 | complement factor H | 143694 | 6.21 | 39% |
| 305 | alpha 2 macroglobulin | 167505 | 6.06 | 9% |
| 305 | inter-alpha-trypsin inhibitor | 106826 | 6.4 | 10% |
| 305 | inter-alpha trypsin inhibitor heavy chain H1 precursor | 101782 | 6.31 | 8% |
| 305 | hemopexin precursor | 52254 | 6.57 | 4% |
| 352 | complement factor H | 143694 | 6.21 | 35% |
| 352 | alpha-2-macroglobulin precursor | 164600 | 6 | 12% |
| 352 | inter-alpha-trypsin inhibitor heavy chain H1 precursor | 101782 | 6.31 | 10% |
| 352 | trypsin inhibitor | 107103 | 6.58 | 9% |
| 352 | complement component C3 | 188585 | 6.02 | 6% |
| 352 | Protein S100-A7 | 11564 | 6.27 | 21% |
| 392 | ceruloplasmin | 116197 | 5.43 | 15% |
| 392 | inter-alpha-trypsin family heavy chain-related protein | 103549 | 6.51 | 15% |
| 392 | phosphatidylinositol-glycan-specific phospholipase D1 | 92943 | 5.91 | 7% |
| | precursor | | | |
| 392 | alpha-2-macroglobulin precursor | 164600 | 6 | 3% |
| 392 | complement C4B | 189599 | 7.39 | 1% |
| 392 | creatine kinase M | 43247 | 6.63 | 3% |
| 396 | ceruloplasmin | 116197 | 5.43 | 19% |
| 396 | inter-alpha-trypsin inhibitor family heavy chain-related | 103549 | 5.43 | 19% |
| | protein | | | |
| 396 | inter-alpha-trypsin family heavy chain-related protein | 103549 | 6.51 | 15% |
| 396 | afamin precursor | 70963 | 5.64 | 15% |
| 396 | C9 complement protein | 64399 | 5.49 | 7% |
| 396 | complement C4B | 189599 | 7.39 | 1% |
| 396 | plasminogen | 93263 | 6.89 | 1% |
| 469 | Chain B, Human Complement Component C3 | 112869 | 5.55 | 27% |
| 469 | ceruloplasmin | 115398 | 5.43 | 5% |
| 469 | complement component 6, isoform CRA_b | 105683 | 6.41 | 6% |
| 469 | hypothetical protein (macroglobulin alpha2 ?) | 163207 | 6 | 28% |
| 509 | inter-alpha-trypsin inhibitor family heavy chain-related | 103321 | 6.51 | 23% |
| | protein | | | |
| 509 | ceruloplasmin | 115398 | 5.43 | 17% |
| 509 | Chain B, Structure of Complement C3b | 103886 | 5.18 | 8% |
| 509 | regucalcin | 33231 | 5.89 | 5% |
| 509 | serpin peptidase inhibitor, clade F | 57016 | 6.47 | 9% |
| 509 | coagulation factor II precursor variant | 70091 | 5.7 | 6% |
| 509 | Chain A, G68a Human Lysozyme | 14705 | 9.28 | 41% |
| 509 | afamin precursor | 69024 | 5.64 | 8% |
| 510 | ceruloplasmin (ferroxidase) | 116685 | 5.48 | 12% |
| 510 | Chain A, Crystal Structure of human Galectin-7 In complex | 14935 | 7 | 27% |
| | with Galactosamine | | | |
| 510 | unnamed protein (S100 calcium-binding protein) | 10931 | 9.19 | 46% |
| 510 | inter-alpha-trypsin inhibitor family heavy chain-related | 103308 | 6.64 | 8% |
| | protein | | | |
| 510 | calmodulin-like skin protein | 15911 | 4.34 | 19% |
| 510 | SCCA2/SCCA1 fusion protein isoform 1 | 44620 | 5.99 | 12% |
| 510 | Protein S100-A7 (Psoriasin) | 11450 | 6.27 | 33% |
| 510 | small proline-rich protein | 8049 | 9.07 | 25% |
| 510 | fatty acid binding protein 5 | 15155 | 6.6 | 40% |
| 510 | tubulin, beta, 4 | 88325 | 5.55 | 6% |
| 512 | ceruloplasmin (ferroxidase) | 116685 | 5.48 | 7% |
| 512 | inter-alpha-trypsin inhibitor family heavy chain-related | 103321 | 6.51 | 9% |
| | protein | | | |
| 547 | inter-alpha-trypsin inhibitor family heavy chain-related | 103321 | 6.51 | 20% |
| | protein | | | |
| 547 | complement component C3 | 187046 | 6.02 | 13% |
| 547 | alpha-2-macroglobulin | 70751 | 5.47 | 16% |
| 547 | ceruloplasmin (ferroxidase) | 116685 | 5.48 | 9% |
| 547 | complement component C3b | 25280 | 4.49 | 16% |
| 547 | fibrinogen gamma | 46252 | 5.54 | 9% |
| 547 | Chain A, Factor H | 136992 | 6.19 | 12% |
| 547 | serum albumin | 69349 | 6.13 | 7% |
| 553 | inter-alpha-trypsin inhibitor family heavy chain-related | 103321 | 6.51 | 22% |
| | protein | | | |
| 553 | ceruloplasmin | 115398 | 5.43 | 15% |
| 553 | complement component C3 | 187046 | 6.02 | 13% |
| 553 | COMP | 82780 | 4.34 | 5% |
| 553 | Chain E, Prethrombin2 | 33789 | 8.32 | 19% |
| 553 | lumican | 38375 | 6.16 | 11% |
| 553 | alpha-2-macroglobulin precursor | 163175 | 6 | 5% |
| 553 | prepro-C3b/C4B | 65725 | 7.72 | 5% |
| 553 | afamin precursor | 69024 | 5.64 | 13% |
| 553 | beta-globin | 18919 | 6.28 | 12% |
| 554 | inter-alpha-trypsin inhibitor family heavy chain-related | 103321 | 6.51 | 16% |
| | protein | | | |
| 554 | complement component C3 | 187046 | 6.02 | 16% |
| 554 | alpha-2-macroglobulin precursor | 163175 | 6 | 9% |
| 554 | ceruloplasmin | 115398 | 5.43 | 7% |
| 554 | annexin A2, isoform CRA_c | 32429 | 5.93 | 16% |
| 554 | alpha-2-antiplasmin precursor | 54194 | 5.71 | 3% |
| 554 | hemopexin precursor | 51512 | 6.57 | 7% |
| 554 | inter-alpha (globulin) inhibitor H1 | 101339 | 6.31 | 9% |
| 555 | complement component C3 | 187046 | 6.02 | 29% |
| 555 | alpha-2-macroglobulin precursor | 163175 | 6 | 11% |
| 555 | inter-alpha-trypsin inhibitor | 106370 | 6.4 | 5% |
| 555 | ceruloplasmin | 115398 | 5.43 | 6% |
| 555 | ALB protein | 71658 | 6.42 | 13% |
| 555 | hemopexin precursor | 51512 | 6.57 | 12% |
| 555 | fibrinogen gamma chain | 47344 | 5.54 | 14% |
| 555 | annexin A2, isoform 2 | 38580 | 7.57 | 8% |
| 555 | alpha-2-anti-plasmin precursor | 54194 | 5.71 | 4% |
| 555 | gelsolin isoform a precursor | 85644 | 5.9 | 3% |
| 561 | complement component C3 | 187046 | 6.02 | 32% |
| 561 | alpha-2-macroglobulin precursor | 163175 | 6 | 12% |
| 561 | trypsin inhibitor | 106647 | 6.58 | 5% |
| 561 | annexin A2, isoform 2 | 38580 | 7.57 | 15% |
| 561 | plasminogen | 57372 | 7.42 | 9% |
| 561 | serum albumin | 69349 | 6.13 | 9% |
| 561 | hemopexin | 51512 | 6.57 | 11% |
| 561 | fibrinogen gamma | 46252 | 5.54 | 7% |
| 561 | complement factor H | 139019 | 6.21 | 5% |
| 567 | Chain B, Human complement component C3 | 112869 | 5.55 | 53% |
| 567 | alpha 2 macroglobulin | 166022 | 6.06 | 10% |
| 567 | hemoglobin beta | 15866 | 5.23 | 14% |
| 567 | hemopexin precursor | 51512 | 6.57 | 13% |
| 568 | complement component C3 | 187046 | 6.02 | 29% |
| 568 | complement factor B | 85450 | 6.55 | 9% |
| 568 | macroglobulin alpha | 160704 | 5.95 | 13% |
| 568 | gelsolin isoforma precursor | 85644 | 5.9 | 8% |
| 568 | S100 calciium-binding protein A9 | 13234 | 5.71 | 19% |
| 655 | complement component 4A preprotein | 192665 | 7.39 | 10% |
| 655 | afamin precursor | 69024 | 5.64 | 27% |
| 655 | ceruloplasmin (ferroxidase) | 116685 | 5.48 | 19% |
| 655 | alpha-2-macroglobulin precursor | 163175 | 6 | 14% |
| 655 | Chain B, Structure of Complement C3b | 103886 | 5.18 | 20% |
| 655 | hypothetical protein inter-alpha (globulin inhibitor H4 | 70808 | 5.86 | 16% |
| 655 | fibulin-1 isoform D precursor | 77223 | 5.11 | 9% |
| 655 | alpha-1-B-glycoprotein | 51908 | 5.65 | 6% |
| 655 | valosin-containing protein | 89266 | 5.14 | 17% |
| 655 | inter-alpha-trypsin inhibitor C-terminal | 93402 | 6.02 | 3% |
| 655 | Vitamin D-binding protein precursor | 52929 | 5.4 | 16% |
| 655 | complement C5 precursor | 188212 | 6.11 | 3% |
| 655 | C9 complement protein | 62974 | 5.49 | 11% |
| 655 | trypsin inhibitor | 106647 | 6.58 | 9% |
| 655 | fibrinogen | 46252 | 5.54 | 8% |
| 655 | serpin B6 (placental thrombin inhibitor) | 42562 | 5.18 | 9% |
| 677 | alpha 2 macroglobulin variant | 164030 | 6 | 16% |
| 677 | complement component C3 | 187046 | 6.02 | 20% |
| 677 | complement factor B | 85450 | 6.55 | 16% |
| 677 | unnamed protein (complement component 2 precursor) | 83180 | 7.23 | 7% |
| 677 | annexin A2 | 38552 | 7.57 | 18% |
| 677 | complement protein C7 precursor | 93453 | 6.09 | 7% |
| 677 | hemopexin precursor | 51512 | 6.57 | 16% |
| 701 | alpha 2 macroglobulin variant | 164030 | 6 | 20% |
| 701 | complement component C3 | 1870046 | 6.02 | 11% |
| 701 | annexin A2 isoform 2 | 38580 | 7.57 | 10% |
| 701 | ANXA1 protein | 5273 | 4.66 | 27% |
| 703 | alpha 2 macroglobulin variant | 164030 | 6 | 16% |
| 703 | complement component 3 precursor | 187030 | 6.02 | 12% |
| 703 | inter-alpha-trypsin inhibitor family heavy chain-related | 103321 | 6.51 | 4% |
| | protein | | | |
| 703 | ceruloplasmin (ferroxidase) | 116685 | 5.48 | 5% |
| 704 | alpha 2 macroglobulin precursor | 164600 | 6 | 9% |
| 704 | hornerin precursor | 188565 | 9.82 | 4% |
| 704 | beta-globin | 19204 | 6.28 | 12% |
| 704 | complement component C3 | 188585 | 6.02 | 1% |
| 704 | cAMP-specific phosphodiesterase | 84945 | 5.03 | 1% |
| 704 | SCP-1 | 114424 | 5.84 | 3% |
| 712 | complement component 1, s subcomponent | 78174 | 4.86 | 21% |
| 712 | inter-alpha-trypsin inhibitor | 99401 | 5.61 | 9% |
| 712 | lumican | 38717 | 6.16 | 24% |
| 712 | ASPIC | 71448 | 4.98 | 12% |
| 712 | afamin precursor | 70963 | 5.64 | 14% |
| 712 | Vitamin K-dependent protein | 77127 | 5.48 | 9% |
| 712 | trypsin inhibitor | 107103 | 6.58 | 5% |
| 712 | lysosomal membrane glycoprotein-2 | 45374 | 5.47 | 1% |
| 712 | gelsolin | 86043 | 5.9 | 2% |
| 712 | Chain L, alpha-thrombin | 4145 | 4.65 | 27% |
| 712 | crystal structure of protein phosphatase 2a | 65173 | 5.07 | 6% |
| 712 | cAMP-specific phospodiesterase | 84945 | 5.03 | 1% |
| 729 | plasma kallikrein precursor (kininogenin) | 73433 | 8.6 | 16% |
| 729 | S100 calcium-binding protein A9 | 13291 | 5.71 | 43% |
| 729 | Chain A, crystal structure of human galectin-7 in complex | 14992 | 7 | 40% |
| | with galactosamine | | | |
| 729 | glyceraldehyde-3-phosphate dehydrogenase | 36202 | 8.26 | 14% |
| 729 | Protein S100-A7 (psoriasin) | 11564 | 6.27 | 23% |
| 729 | stratifin | 27871 | 4.68 | 22% |
| 744 | gelsolin isoform a precursor | 86043 | 5.9 | 22% |
| 744 | alpha-2-macroglobulin precursor | 164600 | 6 | 3% |
| 744 | fibrinogen gamma chain | 50077 | 5.61 | 7% |
| 744 | Chain B, Alpha-Ferrous-Carbonmonoxy | 16070 | 7.12 | 17% |
| 744 | inter-alpha-trypsin inhibitor C-terminal | 93745 | 6.02 | 1% |
| 744 | hemopexin precursor | 52254 | 6.57 | 6% |
| 744 | Complement C1r subcomponent precursor | 81661 | 5.89 | 2% |
| 744 | unnamed protein product (UDP glucuronosyltransferase) | 51813 | 7.62 | 2% |
| 745 | gelsolin isoform a precursor | 86043 | 5.9 | 14% |
| 745 | annexin A2 isoform 2 | 38808 | 7.57 | 9% |
| 745 | alpha 2 macroglobulin | 167505 | 6.06 | 2% |
| 745 | coagulation factor XIII B chain | 77723 | 5.97 | 1% |
| 745 | KIAA1134 protein | 68436 | 5.69 | 6% |
| 748 | gelsolin isoform a precursor | 86043 | 5.9 | 26% |
| 748 | coagulation factor XIII | 77723 | 5.97 | 18% |
| 748 | fibrinogen gamma chain | 50077 | 5.61 | 18% |
| 748 | Chain B, Alpha-Ferrous-Carbonmonoxy, | 15971 | 6.81 | 23% |
| 748 | glucosamine (N-acetyl)-6-sulfatase precursor | 62840 | 8.6 | 6% |
| 748 | annexin A2 isoform | 38808 | 7.57 | 8% |
| 748 | macroglobulin alpha2 | 162072 | 5.95 | 5% |
| 748 | hemopexin precursor | 52254 | 6.57 | 8% |
| 748 | Complement C1r subcomponent precursor | 81661 | 5.89 | 1% |
| 748 | inter-alpha (globulin) inhibitor H1 | 101816 | 6.43 | 4% |
| 763 | afamin precursor | 70963 | 5.64 | 31% |
| 763 | inter-alpha-trypsin inhibitor | 106826 | 6.4 | 14% |
| 763 | coagulation factor II precursor | 71475 | 5.64 | 27% |
| 763 | insulin-like growth factor binding protein, acid labile | 66735 | 6.33 | 18% |
| 763 | inter-alpha (globulin) inhibitor H4 | 103521 | 6.51 | 16% |
| 763 | Chain A, crystal Structure of Lipid-Free Human | 28061 | 5.27 | 35% |
| | Apolipoprotein A-I | | | |
| 763 | alpha-1-B-glycoprotein | 52479 | 5.65 | 17% |
| 763 | histidine-rich glycoprotein precursor | 60510 | 7.09 | 12% |
| 763 | inter-alpha-trypsin inhibitor C-terminal | 93745 | 6.02 | 5% |
| 763 | phospholipid transfer protein isoform a precursor | 54933 | 6.53 | 10% |
| 763 | C9 complement protein | 64399 | 5.49 | 11% |
| 763 | complement C4B precursor | 189599 | 7.39 | 4% |
| 763 | hemopexin precursor | 52254 | 6.57 | 11% |
| 763 | coagulation factor XII | 68618 | 7.94 | 5% |
| 763 | antithrombin III | 53041 | 6.32 | 10% |
| 763 | ceruloplasmin | 98321 | 5.29 | 3% |
| 763 | Chain B, Structure of Complement C3b: Insights Into | | 5.18 | 3% |
| | Complement Activation and Regulation | | | |
| 763 | lumican | 38717 | 6.16 | 9% |
| 763 | serpin peptidase inhibitor clade I | 46287 | 5.08 | 5% |
| 763 | Chain B, Crystal structure of S-Nitroso-Nitrosyl Human | 15922 | 6.81 | 28% |
| | Hemoglobin A | | | |
| 763 | plasma kallikrein precursor | 73433 | 8.6 | 1% |
| 763 | complement component 5 variant | 124357 | 8.43 | 1% |
| 764 | gelsolin isoform a precursor | 86043 | 5.9 | 35% |
| 764 | coagulation factor XIII B chain | 77723 | 5.97 | 23% |
| 764 | glucosamine (N-acetyl)-6-sulfatase precursor | 62840 | 8.6 | 7% |
| 764 | complement C1r subcomponent precursor | 81661 | 81661 | 6% |
| 764 | fibrinogen gamma | 46823 | 5.54 | 7% |
| 764 | cAMP-specific phosphodiesterase | 84945 | 5.03 | 1% |
| 765 | ASPIC | 71448 | 4.98 | 29% |
| 765 | inter-alpha-trypsin inhibitor family heavy chain-related | 103549 | 6.51 | 13% |
| | protein | | | |
| 765 | coagulation factor II precursor (unnamed protein product) | 70723 | 5.53 | 20% |
| 765 | lumican | 38717 | 6.16 | 28% |
| 765 | vitronectin (unnamed protein product) | 55106 | 5.55 | 18% |
| 765 | complement component C4A | 194337 | 6.65 | 3% |
| 765 | histidine-rich glycoprotein precursor | 60510 | 7.09 | 10% |
| 765 | Biotinidase precursor | 59730 | 5.5 | 6% |
| 765 | coagulation factor XII | 68618 | 7.94 | 8% |
| 765 | inter-alpha-trypsin inhibitor heavy chain H3 | 99401 | 5.61 | 4% |
| 765 | plasma kallikrein | 73433 | 8.6 | 5% |
| 765 | alpha-2-antiplasmin precursor | 54536 | 5.71 | 6% |
| 765 | complement component C3 | 188585 | 6.02 | 1% |
| 765 | phospholipid transfer protein isoform a precursor | 54933 | 6.53 | 2% |
| 765 | lysosomal membrane glycoprotein-2 | 45374 | 5.47 | 1% |
| 765 | trypsin inhibitor | 107103 | 6.58 | 5% |
| 765 | inter-alpha-trypsin inhibitor | 93745 | 6.02 | 1% |
| 765 | annexin A2 isoform 2 | 38808 | 7.57 | 2% |
| 765 | preproacrosin | 46455 | 9.2 | 11% |
| 765 | beta globin chain variant | 16117 | 6.75 | 25% |
| 765 | kininogen I | 48936 | 6.29 | 8% |
| 765 | creatine kinase M | 43427 | 6.63 | 3% |
| 765 | serum albumin (unnamed protein) | 71246 | 5.92 | 1% |
| 765 | hemopexin precursor | 52254 | 6.57 | 2% |
| 765 | NLR family member X1 isoform 1 | 108574 | 7 | 2% |
| 766 | afamin precursor | 70963 | 5.64 | 34% |
| 766 | inter-alpha-trypsin inhibitor heavy chain H2 precursor | 106826 | 6.4 | 16% |
| 766 | Chain B, Structure of Complement C3b | 104912 | 5.18 | 14% |
| 766 | coagulation factor II precursor (unnamed protein) | 70723 | 5.53 | 16% |
| 766 | inter-alpha-trypsin inhibitor family heavy chain-related | 103549 | 6.51 | 11% |
| | protein | | | |
| 766 | Chain A, Crystal Structure of Lipid-Free Human | 28061 | 5.27 | 44% |
| | Apolipoprotein A-1 | | | |
| 766 | insulin-like growth factor binding protein, acid labile | 66735 | 6.33 | 17% |
| | subunit | | | |
| 766 | alpha-1-B-glycoprotein | 52479 | 5.65 | 17% |
| 766 | inter-alpha-trypsin inhibitor C-terminal | 93745 | 6.02 | 5% |
| 766 | serine (or cysteine) proteinase inhibitor, clade B | 43004 | 5.61 | 6% |
| 766 | thrombin inhibitor | 42901 | 5.33 | 3% |
| 766 | Complement C5 precursor | 189923 | 6.11 | 3% |
| 766 | histidine-rich glycoprotein precursor | 60510 | 7.09 | 10% |
| 766 | coagulation factor XII | 68618 | 7.94 | 2% |
| 766 | C9 complement protein | 64399 | 5.49 | 1% |
| 766 | lumican | 38717 | 6.16 | 7% |
| 766 | hemopexin precursor | 52254 | 6.57 | 7% |
| 766 | phospholipid transfer protein isoform a precursor | 54933 | 6.53 | 2% |
| 766 | Chain A, Antithrombin Iii | 49350 | 5.95 | 6% |
| 766 | Vitamin D-binding protein precursor | 54526 | 5.4 | 2% |
| 766 | hornerin | 48797 | 9.71 | 6% |
| 770 | gelsolin isoform a precursor | 86043 | 5.9 | 28% |
| 770 | mitosin | 360199 | 5.06 | 0% |
| 770 | macrophin 1 isoform 4 | 603087 | 5.05 | 0% |
| 770 | coagulation factor XIII B chain precursor | 77723 | 5.97 | 5% |
| 770 | ret preprotein | 32219 | 6.06 | 3% |
| 776 | trypsin inhibitor | 107103 | 6.58 | 12% |
| 776 | insulin-like growth factor binding protein | 66735 | 6.33 | 19% |
| 776 | inter-alpha-trypsin inhibitor family heavy chain-related | 103549 | 6.51 | 5% |
| | protein | | | |
| 776 | inter-alpha-trypsin inhibitor C-terminal | 93745 | 6.02 | 5% |
| 776 | ceruloplasmin | 98321 | 5.29 | 2% |
| 776 | Chain L, Alpha-Thrombin | 4145 | 4.65 | 27% |
| 776 | histidine-rich glycoprotein | 60510 | 7.09 | 7% |
| 776 | hemopexin precursor | 52254 | 6.57 | 4% |
| 776 | coagulation factor XII | 68618 | 7.94 | 1% |
| 776 | serine (or cysteine) proteinase inhibitor | 42892 | 5.9 | 6% |
| 776 | proapolipoprotein | 28944 | 5.45 | 8% |
| 776 | cAMP-specific phosphodiesterse | 849945 | 5.03 | 1% |
| 776 | lumican | 38717 | 6.16 | 5% |
| | alpha-1-B-glycoprotein | 52479 | 5.65 | 3% |
| 818 | vanin 1 precursor | 57728 | 5.32 | 13% |
| 818 | coagulation factor II precursor (unnamed protein) | 70723 | 5.53 | 11% |
| 818 | lumican | 38717 | 6.16 | 25% |
| 818 | Biotinidase precursor | 59730 | 5.5 | 4% |
| 818 | alpha-1-antichymotrypsin | 48834 | 5.79 | 14% |
| 818 | vitronectin (unnamed protein) | 55106 | 5.55 | 15% |
| 818 | aggrecan core protein precursor (Cartilage-specific | 251979 | 4.1 | 1% |
| | proteoglycan core protein) | | | |
| 818 | coagulation factor XII | 68618 | 7.94 | 8% |
| 818 | inter-alpha-trypsin inhibitor C-terminal | 93745 | 6.02 | 2% |
| 818 | histidine-rich glycoprotein precursor | 60510 | 7.09 | 5% |
| 818 | ASPIC | 71448 | 4.98 | 5% |
| 818 | plasma protease (C1) inhibitor precursor | 55375 | 6.09 | 9% |
| 818 | lysosomal membrane glycoprotein-2 | 45374 | 5.47 | 1% |
| 818 | Chain B, Structure of Complement C3b | 104912 | 5.18 | 4% |
| 818 | extracellular protein | 45048 | 5.26 | 3% |
| 818 | inter-alpha-trypsin inhibitor family heavy chain-related | 103549 | 6.51 | 4% |
| | protein | | | |
| 818 | trypsin inhibitor | 107103 | 6.58 | 6% |
| 818 | tumor endothelial marker 7-related precursor | 60116 | 5.99 | 4% |
| | COMP precursor | 85403 | 4.34 | 3% |
| 825 | Chain B, Human Complement Component C3 | 114238 | 5.55 | 34% |
| 825 | inter-alpha-trypsin inhibitor heavy chain H! | 101782 | 6.31 | 12% |
| 825 | extracellular matrix protein 1 isoform precursor | 62262 | 6.25 | 16% |
| 825 | alpha-2-macroglobulin precursor protein | 164600 | 6 | 4% |
| 825 | annexin A2 isoform 2 | 38808 | 7.57 | 8% |
| 825 | hemopexin precursor | 52254 | 6.57 | 14% |
| 825 | complement C4B precursor | 189599 | 7.39 | 6% |
| 825 | histidine-rich glycoprotein precursor | 60510 | 7.09 | 5% |
| 825 | gelsolin isoform a precursor | 86043 | 5.9 | 2% |
| 825 | alpha-2-antiplasmin precursor | 54536 | 5.71 | 2% |
| 825 | Dopamine beta-hydroxylase precursor | 68425 | 5.9 | 3% |
| 825 | peptidoglycan recognition protein L precursor | 68699 | 7.62 | 3% |
| 825 | Chain A, Crystal Structure of Native Heparin Cofactor Ii | 55096 | 6.26 | 7% |
| 825 | glucosamine (N-acetyl)-6-sulfatase precursor | 62840 | 8.6 | 4% |
| 825 | beta-globin | 15984 | 7.88 | 8% |
| 827 | lumican | 38717 | 6.16 | 5% |
| 827 | inter-alpha-trypsin inhibitor heavy chain H1 precursor | 101782 | 6.31 | 10% |
| 827 | Chain B, Structure of Complement C3b | 104912 | 5.18 | 23% |
| 827 | Chain A, Crystal Structure of Native Heparin Cofactor Ii | 55096 | 6.26 | 15% |
| 827 | glucosamine (N-acetyl)-6-sulfatase precursor | 62840 | 8.6 | 9% |
| 827 | fibrinogen gamma chain | 50077 | 5.61 | 13% |
| 827 | alpha-2-macroglobulin precursor | 164600 | 6 | 6% |
| 827 | filaggrin 2 | 249296 | 8.45 | 2% |
| 827 | hornerin precursor | 188565 | 9.82 | 5% |
| 827 | annexin A2 isoform 2 | 38808 | 7.57 | 5% |
| 827 | Chain B, T-To-T(High) Quaternary Transitions | 15960 | 6.75 | 21% |
| 827 | hemopexin precursor | 52254 | 6.57 | 8% |
| 827 | gp180-carboxypeptidase D-like enzyme | 153903 | 5.7 | 3% |
| 827 | junction plakoglobin | 82376 | 5.95 | 1% |
| 827 | coagulation factor Xii | 68618 | 7.94 | 1% |
| 833 | desmoplakin I | 334023 | 6.44 | 2% |
| 833 | inter-alpha (globulin) inhibitor H1, isoform CRA_b | 99813 | 6.59 | 19% |
| 833 | complement component C3 | 188585 | 6.02 | 9% |
| 833 | glucosamine (N-acetyl)-6-sulfatase precursor | 62840 | 8.6 | 15% |
| 833 | fibrinogen gamma chain | 50077 | 5.61 | 23% |
| 833 | extracellular matrix protein 1 isoform 1 precursor | 62262 | 6.25 | 5% |
| 833 | alpha-2-macroglobulin precursor | 164600 | 6 | 6% |
| 833 | annexin A2 isoform 2 | 38808 | 7.57 | 8% |
| 833 | gp180-carboxypeptidase D-like enzyme | 153903 | 5.7 | 65 |
| 833 | hemopexin precursor | 52254 | 6.57 | 6% |
| 833 | Chain A, Hr1b Domain from Prk1 | 9054 | 9.77 | 11% |
| 833 | glutathione transferase M3 | 27127 | 5.37 | 4% |
| 844 | matrix, extracellular phosphoglycoprotein with ASARM | 58498 | 8.62 | 11% |
| | motiff | | | |
| 844 | alpha-1-B-glycoprotein | 52479 | 5.65 | 22% |
| 844 | alpha-2-antiplasmin precursor | 54536 | 5.71 | 14% |
| 844 | lumican | 38717 | 6.16 | 28% |
| 844 | inter-alpha-trypsin inhibitor family heavy chain-related | 103549 | 6.51 | 9% |
| | protein | | | |
| 844 | Chain B, Structure of Complement C3b | 104912 | 5.18 | 12% |
| 844 | complement component 1 | 78174 | 4.86 | 15% |
| 844 | hemopexin precursor | 52254 | 6.57 | 14% |
| 844 | Chain B, T-To-T (High) Quaternary Transitions In Human | 15960 | 6.75 | 15% |
| | hemoglobin | | | |
| 844 | histidine-rich glycoprotein precursor | 60510 | 7.09 | 10% |
| 844 | vitronectin precursor (unnamed protein) | 55106 | 5.55 | 11% |
| 844 | coagulation factor II precursor (unnamed protein) | 71475 | 5.64 | 7% |
| 844 | kininogen 1 | 48936 | 6.29 | 8% |
| 844 | preproacrosin | 46455 | 9.2 | 11% |
| 844 | complement component 1 | 54192 | 6.75 | 4% |
| 844 | alpha-2-plasmin inhibitor | 2064 | 4.53 | 63% |
| 844 | Vitamin K-dependent protein S precursor | 77127 | 5.48 | 4% |
| 846 | ASPIC | 71448 | 4.98 | 2% |
| 846 | alpha-1-B-glycoprotein | 52479 | 5.65 | 24% |
| 846 | inter-alpha-trypsin inhibitor | 103549 | 6.51 | 15% |
| 846 | Vitamin K-dependent protein S precursor | 77127 | 5.48 | 22% |
| 846 | Chain B, Human Complement Component C3 | 114238 | 5.55 | 19% |
| 846 | coagulation factor II (unnamed protein) | 70723 | 5.64 | 12% |
| 846 | histidine-rich glycoprotein precursor | 60510 | 7.09 | 13% |
| 846 | Chain B, T-To-T (High) Quaternary Transitions In Human | 15960 | 6.75 | 23% |
| | Hemoglobin | | | |
| 846 | alpha-2-antiplasmin precursor | 54536 | 5.71 | 14% |
| 846 | hemopexin | 52254 | 6.57 | 16% |
| 846 | lumican | 38717 | 6.16 | 7% |
| 846 | complement component C4A | 18042 | 5.08 | 8% |
| 846 | complement 9 | 61728 | 5.42 | 8% |
| 846 | kininogen 1 | 72954 | 6.34 | 4% |
| 846 | inter-alpha-trypsin inhibitor | 93745 | 6.02 | 4% |
| 846 | phospholipid transfer protein isoform a precursor | 54933 | 6.53 | 4% |
| 846 | GRP78 | 72185 | 5.03 | 4% |
| 846 | complement component 1 | 54192 | 6.75 | 4% |
| 846 | Chain A, Hemoglobin Thionville Alpha Chain Mutant with | 15446 | 7.82 | 16% |
| | Val 1 replaced . . . | | | |
| 846 | creatine kinase M | 43247 | 6.63 | 6% |
| 846 | Chain A, Crystal Structure of Native heparin Cofactor Ii | 55096 | 6.26 | 5% |
| 846 | Chain A, Antithrombin Iii | 49350 | 5.95 | 3% |
| 920 | afamin precursor | 70963 | 5.64 | 6% |
| 920 | complement C4B precursor | 189599 | 7.39 | 12% |
| 920 | complement component 3 | 144417 | 8.24 | 12% |
| 920 | inter-alpha-trypsin inhibitor heavy | 101782 | 6.31 | 3% |
| 920 | hemoglobulin beta chain variant Hb S-Wake | 16045 | 7.12 | 23% |
| 920 | glucosamine (N-acetyl)-6-sulfatase precursor | 62840 | 8.6 | 2% |
| 920 | Chain, Annexin I | 35246 | 7.77 | 10% |
| 920 | phospholipase D3 isoform 2 | 49196 | 6 | 2% |
| 920 | hornerin | 48797 | 9.71 | 7% |
| 920 | annexin A2 isoform 2 | 38808 | 7.57 | 7% |
| 920 | butyrophilin | 82036 | 8.4 | 1% |
| 937 | chaperon containing TCP1, subunit 7 isoform a | 59842 | 7.55 | 4% |
| 937 | complement C4B precursor | 189599 | 7.39 | 13% |
| 937 | complement component 3 precursor | 188569 | 6.02 | 10% |
| 937 | annexin A2 isoform 2 | 38808 | 7.57 | 8% |
| 937 | armadillo repeat-containing protein | 31489 | 5.54 | 5% |
| 927 | cAMP-specific phosphodiesterase | 84945 | 5.03 | 3% |
| 972 | kininogen 1 | 48936 | 6.29 | 41% |
| 972 | vitronectin (unnamed protein) | 55099 | 5.55 | 16% |
| 972 | lumican | 38717 | 6.16 | 24% |
| 972 | angiotensinogen | 53407 | 5.78 | 11% |
| 972 | alpha-2-antiplasmin precursor | 54536 | 5.71 | 9% |
| 972 | extracellular protein | 45048 | 5.26 | 17% |
| 972 | ectonucleotide pyrophosphatase | 54745 | 5.94 | 7% |
| 972 | hemopexin precursor | 52254 | 6.57 | 8% |
| 972 | C9 complement protein | 64399 | 5.49 | 14% |
| 972 | carnosinase 1 | 56770 | 5.14 | 9% |
| 972 | complement component C3 | 188585 | 6.02 | 1% |
| 981 | creatine kinase M | 43247 | 6.63 | 3% |
| 981 | complement component 3 | 144417 | 8.24 | 23% |
| 981 | complement C4B precursor | 189599 | 7.39 | 5% |
| 981 | annexin A2 isoform 2 | 38808 | 7.57 | 9% |
| 982 | armadillo repeat-containing protein | 31489 | 5.54 | 5% |
| 982 | complement component 3 | 144417 | 8.24 | 23% |
| 982 | complement C4B precursor | 189599 | 7.39 | 4% |
| 982 | annexin A2 isoform 2 | 38808 | 7.57 | 3% |
| 983 | cAMP-specific phosphodiesterase | 84945 | 5.03 | 1% |
| 983 | kininogen 1 | 48936 | 6.29 | 48% |
| 983 | lumican | 38717 | 6.16 | 18% |
| 983 | alpha-2-antiplasmin precursor | 54536 | 5.71 | 4% |
| 983 | apolipoprotein D | 28317 | 5.14 | 17% |
| 983 | ectonucleotide pyrophosphatase | 54745 | 5.94 | 3% |
| 983 | angiotensinogen | 53407 | 5.78 | 8% |
| 983 | thyroxine-binding globulin precursor | 46637 | 5.87 | 3% |
| 983 | extracellular protein | 45048 | 5.26 | 3% |
| 983 | C9 complement protein | 64399 | 5.49 | 3% |
| 983 | ASPIC | 71448 | 4.98 | 1% |
| 983 | creatine kinase M | 43247 | 6.63 | 3% |
| 983 | cAMP-specific phosphodiesterase | 84945 | 5.03 | 1% |
| 983 | hemopexin precursor | 52254 | 6.57 | 5% |
| 983 | LYST-interacting protein | 26297 | 9.09 | 2% |
| 983 | ubiquitin | 8446 | 6.56 | 12% |
| 983 | alpha-1-antichymotrypsin | 48834 | 5.79 | 3% |
| 990 | complement C8-beta propeptide | 63605 | 8.24 | 3% |
| 990 | complement component 3 | 144417 | 8.24 | 18% |
| 1098 | hornerin | 48797 | 9.71 | 6% |
| 1098 | alpha 2 macroglobulin | 167505 | 6.06 | 5% |
| 1098 | annexin A2 isoform 2 | 38808 | 7.57 | 19% |
| 1098 | plasma carboxypeptidase B2 isoform a preproprotein | 48982 | 7.61 | 6% |
| 1098 | selenoprotein P | 43727 | 7.87 | 13% |
| 1098 | antithrombin III | 53041 | 6.32 | 12% |
| 1098 | kallistatin | 48696 | 7.33 | 11% |
| 1098 | Beta-2-glycoprotein 1 precursor | 39584 | 8.34 | 11% |
| 1098 | hemopexin precursor | 52254 | 6.57 | 10% |
| 1098 | phospholipase D3 isoform | 49196 | 6 | 2% |
| 1098 | glutathione transferase M3 | 27127 | 5.37 | 4% |
| 1098 | calcium binding protein 39 (unnamed protein) | 33844 | 6.67 | 3% |
| 1098 | Chain A, Crystal Structure Analysis of the Bb Segment of | 56816 | 7.16 | 8% |
| | Factor B | | | |
| 1105 | kininogen | 4228 | 6.52 | 34% |
| 1105 | Chain A, antithrombin Iii | 49350 | 5.95 | 47% |
| 1105 | angiotensinogen | 53407 | 5.78 | 19% |
| 1105 | thyroxine-binding globulin precursor | 46637 | 5.87 | 27% |
| 1105 | kininogen 1 | 48936 | 6.29 | 21% |
| 1105 | complement C4B precursor | 189599 | 7.39 | 6% |
| 1105 | alpha-2-antiplasmin precursor | 54536 | 5.71 | 9% |
| 1105 | Vitamin D-binding protein precursor | 54526 | 5.4 | 14% |
| 1105 | blood plasma glutamate carboxypeptidase precursor | 60349 | 8.02 | 5% |
| 1105 | ENPP5 | 54747 | 5.94 | 5% |
| 1105 | trypsin | 27329 | 5.95 | 5% |
| 1105 | complement component C3 | 188585 | 6.02 | 1% |
| 1105 | antithrombin_TRI | 5804 | 4.49 | 18% |
| 1105 | lumican | 38717 | 6.16 | 15% |
| 1105 | alpha 2-plasmin inhibitor | 2064 | 4.53 | 63% |
| 1105 | inter-alpha-trypsin inhibitor | 103549 | 6.51 | 3% |
| 1105 | cAMP-specific phosphodiesterase | 84945 | 5.03 | 1% |
| 1218 | secretoglobin, family 3A member 2 | 10269 | 6.71 | 9% |
| 1218 | blood plasma glutamate carboxypeptidase precursor | 60349 | 8.02 | 3% |
| 1218 | alpha-1-antichymotrypsin | 48834 | 5.79 | 14% |
| 1218 | alpha2-HS glycoproteiin | 40197 | 5.43 | 10% |
| 1244 | creatine kinase M | 43247 | 6.63 | 3% |
| 1244 | alpha-1-antichymotrypsin precursor | 45567 | 5.32 | 26% |
| 1244 | alpha2-HS glycoprotein | 40197 | 5.43 | 16% |
| 1244 | plasma glutamate carboxypeptidase | 52083 | 5.79 | 7% |
| 1244 | Chain A, antithrombin Iii | 49350 | 5.95 | 6% |
| 1244 | creatine kinase M | 43247 | 6.63 | 3% |
| 1244 | hornerin | 48797 | 9.71 | 7% |
| 1244 | COMP | 85403 | 4.34 | 3% |
| 1244 | hematopoietic cell-specific Lyn substrate 1 | | 4.74 | 3% |
| 1244 | chondroitin sulfate proteoglycan 2 | 374585 | 4.45 | 0% |
| 1244 | nesprin 1 longest | 10169979 | 5.37 | 1% |
| 1244 | Rab3-interacting molecule 2 | 161216 | 9.17 | 0% |
| 1297 | ORF | 74466 | 9.49 | 1% |
| 1297 | selenium binding protein | 52928 | 5.93 | 33% |
| 1297 | T-plastin polypeptide | 64281 | 5.73 | 22% |
| 1297 | complement component C3 | 188585 | 6.02 | 4% |
| 1297 | PEPD protein | 55354 | 5.64 | 16% |
| 1297 | ectonucleotide pyrophosphatase/phosphodiesterase 5 | 54745 | 5.94 | 11% |
| 1297 | pigment epithelial-differentiating factor | 46471 | 5.84 | 12% |
| 1297 | annexin A2 isoform 2 | 38808 | 7.57 | 6% |
| 1297 | cd14 protein precursor | 40681 | 5.84 | 4% |
| 1297 | alpha-2-antiplasmin precursor | 54536 | 5.71 | 2% |
| 1297 | glucosamine (N-acetyl)-6-sulfatase precursor | 62840 | 8.6 | 3% |
| 1297 | phospholipase D3 isoform 2 | 49196 | 6 | 2% |
| 1297 | blood plasma glutamate carboxypeptidase precursor | 60349 | 8.02 | 2% |
| 1297 | Chain, The Solution Structure of Reduced monomeric | 15954 | 5.39 | 13% |
| | Superoxide dismutase | | | |
| 1297 | kinesin superfamily protein KIF1B | 200459 | 5.43 | 4% |
| 1297 | lymphoid-restricted membrane protein | 56691 | 5.44 | 3% |
| 1338 | cAMP-specific phosphodiesterase | 84945 | 5.03 | 1% |
| 1338 | phospholipase D3 | 49196 | 6 | 14% |
| 1338 | Chain B, alpha-Ferrous-Carbonmonoxy, Beta-Cobaltous- | 15971 | 6.81 | 32% |
| | Deoxy hemoglobin | | | |
| 1338 | annexin A2 isoform | 38808 | 7.57 | 6% |
| 1338 | lysosomal acid phosphatase 2 precursor | 48713 | 6.28 | 8% |
| 1338 | beta-fibrinogen precursor | 55545 | 8.31 | 7% |
| 1338 | hemoglobin alpha-2 | 15337 | 8.72 | 45% |
| 1338 | plasma serine protease inhibitor precursor | 45886 | 9.38 | 13% |
| 1338 | hemopexin | 13452 | 6.7 | 9% |
| 1338 | regucalcin | 33802 | 5.89 | 6% |
| 1338 | complement component C4A | 194337 | 6.65 | 3% |
| 1338 | complement component C3 | 188585 | 6.02 | 0% |
| 1338 | cAMP-specific phosphodiesterase | 84945 | 5.03 | 1% |
| 1338 | peptidoglycan recognition protein L precursor | 68669 | 7.62 | 1% |
| 1346 | hornerin | 48797 | 9.71 | 7% |
| 1346 | pigment epithelial-differentiating factor | 46471 | 5.84 | 20% |
| 1346 | complement component C3 | 188585 | 6.02 | 5% |
| 1346 | glucosamine (N-acetyl)-6-sulfatase precursor | 62840 | 8.6 | 7% |
| 1346 | fibrinogen gamma chain | 50077 | 5.61 | 12% |
| 1346 | ectonucleotide pyrophosphatase/phosphodiesterase 5 | 54745 | 5.94 | 9% |
| 1346 | beta-fibrinogen precursor | 55545 | 8.31 | 12% |
| 1346 | CFI protein | 44285 | 8.49 | 11% |
| 1346 | aldehyde dehydrogenase | 51367 | 5.83 | 9% |
| 1346 | Chain B, T-To-T (High) Quaternary . . . | 15975 | 6.75 | 15% |
| 1346 | phospholipase D3 isoform 2 | 49196 | 6 | 2% |
| 1346 | gp180-carboxypeptidase D-like enzyme | 153903 | 5.7 | 1% |
| 1346 | annexin A2 isoform | 38808 | 7.57 | 14% |
| 1346 | regucalcin | 33802 | 5.89 | 3% |
| 1346 | plasma serine protease inhibitor precursor | 45886 | 9.38 | 6% |
| 1346 | fetuin-like protein IRL685 | 42922 | 6.87 | 3% |
| 1387 | lactoferrin (unnamed protein) | 80228 | 8.56 | 1% |
| 1387 | coagulation factor X precursor | 53870 | 5.34 | 16% |
| 1387 | ceruloplasmin | 116197 | 5.43 | 2% |
| 1387 | alpha-2-antiplasmin precursor | 54536 | 5.71 | 4% |
| 1387 | complement C4B precursor | 189599 | 7.39 | 5% |
| 1387 | alpha-N-acetylgalatosaminidase | 40506 | 5.34 | 3% |
| 1387 | complement component C3 | 188585 | 6.02 | 2% |
| 1387 | serum paraoxonase/arylesterase 1 | 39895 | 5.08 | 6% |
| 1387 | antithrombin III | 53041 | 6.32 | 4% |
| 1387 | nesprin 1 isoform longer | 1011226 | 5.37 | 0% |
| 1397 | creatine kinase M | 43247 | 6.63 | 3% |
| 1397 | hemopexin precursor | 52254 | 6.57 | 12% |
| 1397 | Chain B, Alpha-Ferrous-Carbonmonoxy | 15971 | 6.81 | 31% |
| 1397 | annexin A2 precursor | 38808 | 7.57 | 6% |
| 1414 | cAMP-specific phosphodiesterase | 84945 | 5.03 | 2% |
| 1414 | hemopexin precursor | 52254 | 6.57 | 26% |
| 1414 | 2-phosphopyruvate-hydratase alpha-enolase | 47421 | 7.01 | 21% |
| 1414 | annexin A2 isoform 2 | 38808 | 7.57 | 8% |
| 1414 | alpha 2 macroglobulin | 167505 | 6.06 | 2% |
| 1414 | arylsulfatase B precursor | 60195 | 8.43 | 3% |
| 1439 | complement component C3 | 188585 | 6.02 | 0% |
| 1439 | complement component 4 binding protein | 29308 | 5.05 | 8% |
| 1439 | Chain B, Structure of Complement C3b | 104912 | 5.18 | 4% |
| 1439 | protein, alpha1 acid glyco | 21433 | 5.09 | 9% |
| 1439 | complement C4B precursor | 189599 | 7.39 | 2% |
| 1439 | integrin beta-1 precursor | 91714 | 5.3 | 1% |
| 1447 | ankyrin-3 | 482387 | 6.12 | 0% |
| 1447 | complement component C3 | 188585 | 6.02 | 7% |
| 1447 | Chain B, Crystal Structure of S-Nitroso-Nitrosyl | 15922 | 6.81 | 53% |
| | Hemoglobin A | | | |
| 1447 | annexin A2 isoform 2 | 38808 | 7.57 | 16% |
| 1447 | arylsulfatase B precursor | 60195 | 8.43 | 6% |
| 1447 | lysophospholipase 3 | 46913 | 6.27 | 13% |
| 1447 | Chain annexin I | 35246 | 7.77 | 5% |
| 1447 | phospholipase D3 | 49196 | 6 | 2% |
| 1503 | alpha-fibrinogen precursor | 70223 | 8.26 | 14% |
| 1503 | Chain B, Alpha-Ferrous-Carbonmonoxy | 15971 | 6.81 | 31% |
| 1503 | annexin A2 isoform 2 | 38808 | 7.57 | 16% |
| 1503 | neuropolypeptide h3 | 21027 | 7.42 | 20% |
| 1503 | glucosamine (N-acetyl0-6-sulfatase precursor | 62840 | 8.6 | 4% |
| 1503 | phospholipase D3 isoform 2 | 49196 | 6 | 4% |
| 1503 | Chain E, Structure of Human Transferrin Receptor- | 39476 | 6.41 | 6% |
| | Transferrin Complex | | | |
| 1503 | cAMP-specific phosphodiesterase | 84945 | 5.03 | 2% |
| 1503 | dismutase, Cu/Zn superoxide | 16020 | 8.76 | 7% |
| 1503 | complement component C3 | 188585 | 6.02 | 1% |
| 1503 | glutathione transferase M3 | 27127 | 5.37 | 7% |
| 1563 | Rho guanine nucleotide exchange factor | 223645 | 5.9 | 0% |
| 1563 | mutant beta-actin | 42128 | 5.22 | 16% |
| 1563 | thrombin inhibitor | 42901 | 5.33 | 2% |
| 1563 | ectonucleotide pyrophosphatase/phosphodiesterase 5 | 54745 | 5.94 | 6% |
| 1563 | complement C4B precursor | 189599 | 7.39 | 4% |
| 1656 | Vitamin D-binding protein precursor | 54256 | 5.4 | 2% |
| 1656 | prepro-C3b/C4B inactivator | 68120 | 7.72 | 5% |
| 1656 | ectonucleotide pyrophosphatase/phosphodiesterase 5 | 54745 | 5.94 | 11% |
| 1656 | annexin A2 isoform 2 | 38808 | 7.57 | 14% |
| 1656 | apolipoprotein J precursor | 49342 | 6.27 | 12% |
| 1656 | regucalcin | 33802 | 5.89 | 12% |
| 1656 | phospholipase D3 isoform | 49196 | 6 | 2% |
| 1656 | thrombin inhibitor | 42901 | 5.33 | 2% |
| 1656 | Serpin B8 (Cytoplasmic antiproteinase 2) | 43328 | 5.43 | 2% |
| 1656 | Chain, Glutathione S-Transferase | 25729 | 6.02 | 12% |
| 1656 | inter-alpha-trypsin inhibitor family heavy chain-related | 103549 | 6.51 | 1% |
| | protein | | | |
| 1656 | cAMP-specific phosphodiesterase | 84945 | 5.03 | 1% |
| 1671 | HRX | 436238 | 9.23 | 0% |
| 1671 | antithrombin III | 53041 | 6.32 | 6% |
| 1671 | ectonucleotide pyrophosphatase/phosphodiesterase 5 | 54745 | 5.94 | 2% |
| 1671 | alpha2-HS glycoprotein | 36268 | 5.2 | 2% |
| 1671 | creatine kinase M | 43247 | 6.63 | 3% |
| 1671 | cAMP-specific phophodiesterase | 84945 | 5.03 | 3% |
| 1671 | apolipoprotein D | 28317 | 5.14 | 15% |
| 1671 | mitochondrial ribosomal protein L30 | 18678 | 10.01 | 5% |
| 1671 | HER2 receptor | 141145 | 5.58 | 1% |
| 1681 | Mucin-16 (Ovarian carcinoma antigen CA125) | 2359682 | 5.65 | 0% |
| 1681 | apolipoprotein J precursor | 49342 | 6.27 | 24% |
| 1681 | complement component C3 | 188585 | 6.02 | 4% |
| 1681 | beta-globin types (unnamed protein) | 16070 | 7.12 | 8% |
| 1681 | annexin A2 isoform | 38808 | 7.57 | 6% |
| 1681 | prepro-C3b/C4B inactivator | 68120 | 7.72 | 4% |
| 1681 | regucalcin | 33802 | 5.89 | 12% |
| 1681 | creatine kinase M | 43247 | 6.63 | 3% |
| 1689 | cAMP-specific phophodiesterase | 84945 | 5.03 | 2% |
| 1689 | Chain B, Structure of Complement C3b | 104912 | 5.18 | 7% |
| 1689 | hypothetical protein | 53029 | 5.89 | 14% |
| 1689 | prepro-C3b/C4B inactivator | 68120 | 7.72 | 1% |
| 1689 | regucalcin | 33802 | 5.89 | 3% |
| 1690 | cAMP-specific phophodiesterase | 84945 | 5.03 | 1% |
| 1690 | annexin A2 isoform 2 | 38808 | 7.57 | 22% |
| 1690 | transaldolase 1 | 37688 | 6.36 | 13% |
| 1690 | Chain B, Cathepsin D | 26457 | 5.31 | 7% |
| 1690 | plasminogen | 93263 | 6.89 | 1% |
| 1690 | Chain A, Crystal Structure of homo sapien glycerol-3- | 38401 | 5.81 | 5% |
| | phosphate dehydrogenase 1 | | | |
| 1690 | phosphodiesterase 4D, cAMP-specific . . . | 24806 | 9.88 | 12% |
| 1690 | Chain B, Hemoglobin (Deoxy) Mutant with Val B . . . | 15866 | 6.7 | 22% |
| 1719 | glutathione transferase M3 | 27127 | 5.37 | 4% |
| 1719 | apolipoprotein J precursor | 49342 | 6.27 | 20% |
| 1725 | plasminogen | 93263 | 6.89 | 1% |
| 1725 | transaldolase 1 | 37688 | 6.36 | 13% |
| 1725 | annexin A2 isoform 2 | 38808 | 7.57 | 22% |
| 1725 | Chain B, Alpha-Ferrous-Carbonmonoxy, . . . | 15971 | 6.81 | 22% |
| 1725 | glutathione S-transferase GSTMS-5 | 25847 | 6.9 | 9% |
| 1725 | plasminogen | 93233 | 7.04 | 0% |
| 1737 | malate dehydrogenase, mitochondrial precursor | 35965 | 8.92 | 10% |
| 1737 | regucalcin | 33802 | 5.89 | 27% |
| 1737 | immunoglobulin lambda light chain VLJ region | 11755 | 8.62 | 9% |
| 1737 | bridging integrator 3 | 29703 | 6.95 | 4% |
| 1745 | cAMP-specific phosphodiesterase | 84945 | 5.03 | 2% |
| 1745 | regucalcin | 33802 | 5.89 | 17% |
| 1745 | apolipoprotein D | 28317 | 5.14 | 25% |
| 1745 | apolipoprotein J precursor | 49342 | 6.27 | 6% |
| 1745 | annexin A2 isoform 2 | 38808 | 7.57 | 6% |
| 1745 | antithrombin III | 53041 | 6.32 | 8% |
| 1745 | Rho guanine nucleotide exchange factor | 223645 | 5.9 | 2% |
| 1747 | alpha2-HS glycoprotein | 36268 | 5.2 | 2% |
| 1747 | apolipoprotein D | 28317 | 5.14 | 20% |
| 1747 | regucalcin | 4759036 | 5.89 | 11% |
| 1747 | annexin A2, isoform | 32600 | 5.93 | 9% |
| 1747 | apolipoprotein J precursor | 49342 | 6.27 | 4% |
| 1757 | cAMP-specific phosphodiesterase | 84945 | 5.03 | 1% |
| 1757 | regucalcin | 33802 | 5.89 | 11% |
| 1757 | protein, alpha1 acid glyco | 21443 | 5.09 | 4% |
| 1757 | apolipoprotein J precursor | 49342 | 6.27 | 6% |
| 1757 | cAMP-specific phosphodiesterase | 84945 | 5.03 | 1% |
| 1757 | annexin A2 isoform 2 | 38808 | 7.57 | 3% |
| 1778 | aberrant LSLCL | 33536 | 6.82 | 6% |
| 1778 | annexin A2 isoform 2 | 38808 | 7.57 | 43% |
| 1778 | Chain, Annexin Family Mol_{—} | 36480 | 5.63 | 13% |
| 1778 | ficolin 3 isoform 1 precursor | 66198 | 6.2 | 9% |
| 1778 | alpha-fibrinogen precursor | 70223 | 8.26 | 4% |
| 1778 | Chain B, Cathepsin D | 26457 | 5.31 | 7% |
| 1778 | plasminogen | 93233 | 7.04 | 0% |
| 1778 | cAMP-specific phosphodiesterase PDE4D5 | 84945 | 5.03 | 1% |
| 1778 | actin related protein2/3 | 34426 | 6.84 | 3% |
| 1778 | basic helix-loop-helix domain containing | 23935 | 9.37 | 3% |
| 1778 | mitochondrial ribosomal protein S34 | 25692 | 9.98 | 3% |
| 1778 | Bruton's agammaglobulinemia tyrosine kinase | 76625 | 8.05 | 1% |
| 1781 | ubinuclein | 122036 | 9.37 | 2% |
| 1781 | Chain, Human Annexin V with Proline substitution by | 36041 | 4.94 | 19% |
| | Thioproline | | | |
| 1781 | annexin A2 isoform 2 | 38808 | 7.57 | 18% |
| 1781 | Chain, Annexin Family Mol_{—} | 36480 | 5.63 | 6% |
| 1781 | cathepsin Z precursor | 34544 | 6.7 | 3% |
| 1781 | complex-forming glycoprotein HC | 20592 | 5.84 | 11% |
| 1781 | apolipoprotein J precursor | 49342 | 6.27 | 5% |
| 1781 | cAMP-specific phosphodiesterase | 84945 | 5.03 | 1% |
| 1781 | bromodomain adjacent to zinc finger domain 2B | 222440 | 5.95 | 1% |
| 1783 | mitochondrial ribosomal protein L30 | 18678 | 10.01 | 10% |
| 1783 | annexin A2 isoform 2 | 38808 | 7.57 | 27% |
| 1783 | Chain, Annexin Family Mol | 36480 | 5.63 | 16% |
| 1783 | alpha-fibrinogen precursor | 70223 | 8.26 | 2% |
| 1783 | annexin II cell-surface form = cytomegalovirus binding | 2160 | 5.8 | 100% |
| | protein | | | |
| 1783 | Chain B, Cathepsin D | 26457 | 5.31 | 7% |
| 1783 | Chain, Annexin I | 35246 | 7.77 | 5% |
| 1783 | apolipoprotein J precursor | 49342 | 6.27 | 6% |
| 1783 | glutathione transferase M3 | 27127 | 5.37 | 4% |
| 1783 | truncated putative T7-like mitochondrial DNA helicase | 66430 | 8.33 | 5% |
| 1783 | cAMP-specific posphodiesterase | 84945 | 5.03 | 1% |
| 1783 | alpha-2-macroglobulin precursor | 164600 | 6 | 0% |
| 1791 | Chain A, Hr1b Domain From Prk1 | 9054 | 9.77 | 11% |
| 1791 | annexin A2 isoform 2 | 38808 | 7.57 | 32% |
| 1791 | Chain, Annexin Family Mol_{—} . . . | 36480 | 5.63 | 13% |
| 1791 | Chain, Annexin I | 35246 | 7.77 | 7% |
| 1791 | annexin II cell-surface form = . . . | 2160 | 5.8 | 100% |
| 1791 | complement C4B precursor | 189599 | 7.39 | 2% |
| 1865 | Chain B, Cathepsin D | 26457 | 5.31 | 7% |
| 1865 | protein PP4-X (annexin variant) | 36262 | 5.65 | 18% |
| 1868 | cAMP-specific phosphodiesterase | 84945 | 5.03 | 4% |
| 1868 | protein PP4-X (annexin A4 variable) | 36262 | 5.65 | 26% |
| 1868 | apolipoprotein E | 36302 | 5.65 | 5% |
| 1868 | Chain B, Crystal Structure of S-Nitroso-Nitrosyl Human | 15922 | 6.81 | 23% |
| | hemoglobin A | | | |
| 1868 | regucalcin | 33802 | 5.89 | 10% |
| 1868 | protein disulfide isomerase-related protein 5 | | 4.95 | 2% |
| 1868 | cathepsin Z precursor | 34544 | 6.7 | 3% |
| 1868 | serine (or cysteine) proteinase inhibitor | 42829 | 5.9 | 1% |
| 1868 | Chain C, X-Ray Crystal Structure of Cyclophilin AHIV-1 . . . | 16178 | 5.94 | 4% |
| 1868 | Chain A, Crystal Structure of Human Tr . . . | 30705 | 5.41 | 4% |
| 1868 | coiled-coil domain containing 96 | 62958 | 4.92 | 1% |
| 1915 | immunoglobulin lambda light chain VLJ region . . . | 11755 | 8.62 | 9% |
| 1915 | T-plastin polypeptide (actin crosslinking) | 64281 | 5.73 | 12% |
| 1915 | Chain A, Crystal Structure of Lipid-Free Human | 28061 | 5.27 | 44% |
| | Apolipoprotein A-I | | | |
| 1915 | nuclear chloride channel | 27249 | 5.02 | 20% |
| 1915 | regucalcin | 33802 | 5.89 | 18% |
| 1915 | inositol-1-monophosphatase | 26813 | 5.74 | 10% |
| 1915 | Chain B, T-To-T Quaternary Transitions . . . | 15927 | 6.75 | 30% |
| 1915 | thrombin inhibitor | 42901 | 5.33 | 3% |
| 1915 | UCC1 protein (mammalian ependymin protein) | 20162 | 5.03 | 6% |
| 1915 | ectonucleotide pyrophosphatase/phosphodiesterase | 54745 | 5.94 | 1% |
| 1915 | glutathione S-transferase | 25847 | 6.9 | 11% |
| 1915 | complement C4B precursor | 189599 | 7.39 | 3% |
| 1915 | protein disulfide isomerase-related protein 5 | 46512 | 4.95 | 2% |
| 1915 | complex-forming glycoprotein HC | 20592 | 5.84 | 7% |
| 1915 | Chain A, Hemoglobin Thionville Alpha Chain Mutant . . . | 15446 | 7.82 | 23% |
| 1915 | Chain B, Cathepsin D | 26457 | 5.31 | 7% |
| 1915 | gelsolin isoform a precursor | 86043 | 5.9 | 1% |
| 1915 | albumin, isoform CRA_a | 25732 | 6.45 | 3% |
| 1915 | heat shock 70 kDa protein 8 isoform 1 | 71082 | 5.37 | 1% |
| 1915 | serpin pieptidase inhibitor, clade I . . . | 46287 | 5.08 | 6% |
| 1915 | cAMP-specific phosphodiesterase | 84945 | 5.03 | 2% |
| 1915 | complement factor B | 86819 | 6.55 | 0% |
| 1915 | heat shock protein | 70237 | 5.56 | 1% |
| 1915 | cathepsin F | 38244 | 6.54 | 2% |
| 1938 | thrombospondin-4 | 108415 | 4.44 | 1% |
| 1938 | apolipoprotein D | 28317 | 5.14 | 29% |
| 1938 | 78 kDa glucose regulated protein precursor | 72492 | 5.07 | 8% |
| 1938 | L-plastin | 70815 | 5.2 | 8% |
| 1938 | heat shock 70 kDa protein 8 isoform 1 | 71082 | 5.37 | 6% |
| 1938 | Chain B, Crystal Structure of S-Nitroso-Nirosyl Human | 15922 | 6.81 | 23% |
| | Hemoglobin A | | | |
| 1938 | beta-trace protein, prostaglandin D synthase | 18898 | 6.95 | 4% |
| 1938 | glutathione transferase M3 | 27127 | 5.37 | 4% |
| 2029 | creatine kinase M | 43247 | 6.63 | 7% |
| 2029 | heat shock 70 kDa protein 8 isoform 1 | 71082 | 5.37 | 8% |
| 2029 | heat shock protein | 70237 | 5.56 | 9% |
| 2029 | GRP78 (78 kDa glucose-regulated protein precursor) | 72185 | 5.03 | 7% |
| 2029 | proapolipoprotein | 28944 | 5.45 | 20% |
| 2029 | glutathione transferase M3 | 27127 | 5.37 | 4% |
| 2029 | Chain A, Human Aspartylglucosaminidase | 17552 | 4.82 | 4% |
| 2029 | HSPC336 (apolipoprotein M) | 21220 | 6.48 | 3% |
| 2052 | cAMP-specific phosphodiesterase | 84945 | 5.03 | 2% |
| 2052 | heat shock 70 kDa protein 8 isoform | 71082 | 5.37 | 7% |
| 2052 | heat shock protein | 70237 | 5.56 | 7% |
| 2052 | GRP78 precursor (78 kDa glucose-regulated protein | 72185 | 5.03 | 5% |
| | precursor | | | |
| 2052 | lipoprotein Gln I | 28329 | 5.27 | 16% |
| 2052 | albumin, isoform CRA_t | 60211 | 6.66 | 3% |
| 2052 | glutathione transferase M3 | 27127 | 5.37 | 11% |
| 2052 | beta-trace protein, prostaglandin D synthase | 18898 | 6.95 | 4% |
| 2068 | plasminogen | 93263 | 6.89 | 1% |
| 2068 | Chain A, Human aspartylglucosaminidase | 17552 | 4.82 | 9% |
| 2068 | 78 kDa glucose-regulated protein precursor | 72492 | 5.07 | 6% |
| 2068 | heat shock 70 kDa protein 8 isoform 1 | 71082 | 5.37 | 2% |
| 2068 | cAMP--specific phosphodiesterase | 84945 | 5.03 | 1% |
| 2090 | proapolipoprotein | 28944 | 5.45 | 3% |
| 2090 | Chain B, Non-Covalent Complex Between Alpha-1-Pi . . . | 26077 | 8.23 | 51% |
| 2090 | Chain C, Globular Head of the Complement System Protein | 14451 | 8.85 | 25% |
| | C1q | | | |
| 2090 | putative acrosin-like protease | 25468 | 9.09 | 6% |
| 2090 | 2′,′3′-cyclic-nucleotide 3′-phosphodiesterase | 45469 | 8.73 | 4% |
| 2090 | Chain A, Trypsin | 24669 | 7.64 | 4% |
| 2090 | alpha-fibrinogen precursor | 70223 | 8.26 | 2% |
| 2090 | glutathione transferase A5 | 25763 | 7.74 | 6% |
| 2090 | inter-alpha-trypsin inhibitor | 93745 | 6.02 | 1% |
| 2090 | collagen alpha 1 | 70610 | 9.18 | 2% |
| 2090 | immunoglobulin lambda-chain | 13716 | 7.71 | 10% |
| 2090 | cAMP-specific phosphodiesterase | 84945 | 5.03 | 4% |
| 2146 | potassium channel, subfamily K, member 18 | 44498 | 6.6 | 1% |
| 2146 | glutathione peroxidase 3 precursor | 25774 | 8.2 | 27% |
| 2146 | human basement membrane heparan sulfate proteoglycan | 479812 | 6.1 | 0% |
| | core protein | | | |
| 2146 | mitochondrial ribosomal protein L30 | 18678 | 10.01 | 5% |
| 2146 | calcium channel beta-2 chain | 53073 | 9.3 | 4% |
| 2180 | cAMP-specific phosphodiesterase | 84945 | 5.03 | 1% |
| 2180 | glucosamine-phosphate N-acetyltransferase 1 | 21078 | 8.17 | 21% |
| 2180 | neuropolypeptide h3 | 21027 | 7.42 | 26% |
| 2180 | preproacrosin | 46455 | 9.2 | 3% |
| 2180 | cAMP-specific phosphodiesterase | 84945 | 5.03 | 4% |
| 2208 | axonemal dynein light chain 1 | 17256 | 5.32 | 5% |
| 2208 | Chain B, T-To-T(High) Quaternary Transitions | 15911 | 6.75 | 28% |
| 2208 | cytoplasmic phosphotyrosyl protein phosphatase | 18091 | 7.04 | 12% |
| 2208 | Chain, The Solution Structure of Reduced Monomericc | 15954 | 5.39 | 13% |
| | Superoxide Dismutase | | | |
| 2208 | Chain B, Crystal Structure of A Rac-Rhogdi Complex | 20521 | 6.16 | 8% |
| 2208 | albumin, isoform CRA_b | 61122 | 6.96 | 4% |
| 2215 | plasminogen | 93263 | 6.89 | 1% |
| 2215 | DOK6 protein (docking protein 5-like | 31492 | 5.9 | 4% |
| 2215 | dermcidin preproprotein | 11391 | 6.08 | 10% |
| 2235 | shroom family member 3 protein | 218190 | 7.87 | 3% |
| 2235 | aspartylglucosaminidase alpha subunit (N-terminal) | 2177 | 8.53 | 40% |
| 2235 | ubiquitin-conjugating enzyme 9 | 18146 | 8.67 | 5% |

TABLE 5

| | |
|---|---|
| 27 | alpha-2-macroglobulin precursor |
| 76 | fibronectin precursor, Chain B, Structure of complement C3b, C9 |
| | complement protein |
| 93 | no proteins meet the criteria |
| 124 | unnamed protein (coagulation factor II precursor) |
| 206 | alpha-1-antichymotrypsin |
| 210 | alpha-2-macroglobulin precursor, complement component C3, complement |
| | factor H |
| 246 | alpha-2-macroglobulin precursor, complement factor H |
| 255 | macroglobulin alpha2, complement factor H |
| 265 | No proteins meet criteria |
| 292 | alpha-2-macroglobulin precursor, complement factor H, trypsin inhibitor, |
| | inter-alpha-trypsin heavy chain H1 precursor |
| 372 | alpha-2-macroglobulin precursor, complement factor H isoforma precursor, |
| | gelsolin isoform a precursor |
| 384 | Ceruloplasmin, inter-alpha-trypsin inhibitor heavy chain H4 precursor, |
| | phosphatidylinositol-glycan-specific phospholipase D1 precursor |
| 385 | phosphatidylinositol-glycan-specific phospholipase D1 precursor, |
| | ceruloplasmin, inter-alpha-trypsin inhibitor family heavy chain-related |
| | protein |
| 393 | Ceruloplasmin, inter-alpha-trypsin inhibitor heavy chain H4 precursor, |
| | phosphatidylinositol-glycan-specific phospholipase D1 precursor |
| 394 | inter-alpha-trypsin inhibitor heavy chain H4 precursor, ceruloplasmin |
| 408 | glycosylphosphatidylinositol specific phosphatase D1, ceruloplasmin |
| 477 | alpha-2-macroglobulin precursor, complement component C6 precursor |
| | peptide, complement factor B |
| 481 | pre-pro-alpha(I) collagen |
| 495 | Ceruloplasmin, complement component C3, factor H, SERPIN2 protein, gp- |
| | 180-carboxypeptidase D-like enzyme |
| 496 | inter-alpha-trypsin inhibitor family heavy chain-related protein, Chain B, Human |
| | Complement Component C3, alpha-2-macroglobulin precursor, ceruloplasmin |
| 644 | Fibulin-1 isoform D precursor, inter-alpha-trypsin inhibitor family heavy |
| | chain-related protein, plasminogen, complement factor B, HGF activator, |
| | preproprotein |
| 746 | unnamed protein (coagulation factor II precursor), afamin precursor, |
| | Vitamin K-dependent protein, complement component 1, s subcomponent, |
| | iinter-alpha-trypsin inhibitor, ASPIC |
| 805 | complement component 3 precursor, plasma kallikrein precursor, annexin |
| | A2 isoform 2 |
| 840 | glucosamine (N-acetyl)-6-sulfatase precursor, unnamed protein (cystic |
| | fibrosis antigen), ezrin (p81) (cytovillin) (villin-2), S100 Calcium binding |
| | protein A9 |
| 849 | glucosamine (N-acetyl)-6-sulfatase precursor, coagulation factor XIII A |
| | chain precursor, peptidoglycan recognition protein L precursor, complement |
| | component 4 binding protein |
| 856 | inter-alpha-trypsin inhibitor heavy chain H1 precursor, glucosamine (N- |
| | acetyl)-6-sulfatase precursor, fibrinogen gamma chain, coagulation factor |
| | XIII A chain precursor |
| 864 | glucosamine (N-acetyl)-6-sulfatase precursor, Ig mu chain precursor, |
| | phospholipase D3 isoform, moesin |
| 962 | complement 9, Chain B, Structure of Complement C3b, alpha-2-antiplasmin |
| | precursor, kininogen, thrombin inhibitor |
| 973 | complement 9, Chain A, antithrombin Iii, alpha-2-antiplasmin precursor, |
| | kinninogen, thrombin inhibitor, L-plastin, complement component 1, alpha- |
| | 1-B-glycoprotein, hemopexin precursor |
| 1472 | complement component C4A, complement component C3, apolipoprotein |
| | A-IV precursor, serum paraoxonase/arylesterase, preprohaptoglobulin, |
| | COMP, serpin peptidase inhibitor, clade I, follistatin-like 1 precursor |
| 1498 | complement factor H-related protein 1 precursor, Chain A, Crystal Structure |
| | of Lipid-Free human Apolipoprotein A-1, annexin A2 isoform 2, |
| | phospholipase D3 isoform 2, Chain E, Structure of human transferring |
| | receptor-transferrin complex |
| 44 | fibronectin precursor, complement component C3, alpha-2-macroglobulin |
| | precursor |
| 82 | Chain B, Structure of Complement C3b |
| 109 | factor H, fibronectin precursor |
| 126 | fibronectin 1 isoform 3 preproprotein, alpha-2-macroglobulin precursor, |
| 164 | macroglobulin alpha 2 |
| 184 | alpha-2-macroglobulin |
| 191 | alpha-2-macroglobulin, complement component C3 |
| 205 | no protein matches criteria |
| 216 | collagen type IV alpha 1, alpha 2 type IV collagen preproprotein |
| 244 | collagen type IV alpha 1, alpha 2 type IV collagen preproprotein |
| 252 | alpha 2 macroglobulin, complement factor H, complement component C3 |
| 267 | inter-alpha-trypsin inhibitor, Human Factor H, fibronectin precursor, inter- |
| | alpha-trypsin inhibitor, C-terminal |
| 295 | complement factor H, alpha 2 macroglobulin, trypsin inhibitor, inter-alpha- |
| | trypsin inhibitor |
| 352 | complement factor H, alpha 2 macroglobulin, inter-alpha-trypsin inhibitor, |
| | trypsin inhibitor, complement component C3 |
| 392 | Ceruloplasmin, phosphatidylinositol-glycan-specific phospholipase D1 |
| 396 | afamin precursor, ceruloplasmin, inter-alpha-trypsin inhibitor |
| 469 | complement component 6, isoform CRA_b |
| 509 | inter-alpha-trypsin inhibitor heavy chain-related protein, ceruloplasmin, |
| | Chain B, Structure of Complement C3b |
| 510 | ceruloplasmin (ferroxidase), Chain A, Crystal Structure of human Galectin-7 |
| 512 | ceruloplasmin (ferroxidase), inter-alpha-trypsin inhibitor family heavy |
| | chain-related protein |
| 547 | serum albumin, inter-alpha-trypsin inhibitor family heavy chain-related |
| | protein |
| 533 | COMP, ceruloplasmin |
| 554 | inter-alpha-trypsin inhibitor family heavy chain-related protein |
| 555 | ALB protein, gelsolin isoform a precursor |
| 561 | serum albumin, trypsin inhibitor, complement component C3 |
| 567 | Chain B, Human complement component C3 |
| 568 | complement component C3, complement factor B, gelsolin isoform |
| | precursor |
| 655 | Ceruloplasmin (ferroxidase), fibulin-1 isoform D precursor, hypothetical |
| | protein (inter-alpha-globulin inhibitor H4), valosin-containing protein, |
| | Vitamin D-binding protein precursor |
| 677 | unnamed protein (complement component 2 precursor), annexin A2, |
| | complement factor B |
| 701 | Annexin A2 isoform 2 |
| 703 | inter-alpha-trypsin inhibitor family heavy chain-related protein, |
| | ceruloplasmin (ferroxidase) |
| 704 | hornerin precursor |
| 712 | complement component 1, s subcomponent, ASPIC, afamin precursor, |
| | Vitamin K-dependent protein |
| 729 | plasma kallikrein precursor, S100 calcium-binding protein A9, Protein |
| | S100-A7 (psoriasin) |
| 744 | gelsolin isoform a precursor |
| 745 | gelsolin isoform a precursor |
| 748 | gelsolin isoform a precursor, coagulation factor XIII, Chain b, Alpha- |
| | Ferrous-Carbonmonoxy |
| 763 | afamin precursor, coagulation factor II precursor, insulin-like growth factor |
| | binding protein, acid labile, histidine-rich glycoprotein precursor, |
| | phospholipid transfer protein isoform a precursor, antithrombin III |
| 764 | glucosamine (N-acetyl)-6-sulfatase precursor, coagulation factor XIII B |
| | chain, gelsolin isoform a precursor |
| 765 | ASPIC, coagulation factor II precursor (unnamed protein), vitronectin |
| | (unnamed protein), histidine-rich glycoprotein precursor, biotinidase |
| | precursor |
| 766 | afamin precursor, insulin-like growth factor binding protein, acid labile |
| | subunit, coagulation factor II precursor, alpha-1-B-glycoprotein, thrombin |
| | inhibitor, C9 complement protein |
| 770 | Coagulation factor XIII B chain precursor |
| 776 | insulin-like growth factor binding protein, hemopexin precursor |
| 818 | vanin 1 precursor, biotinidase precursor, vitronectin (unnamed protein), |
| | ASPIC |
| 825 | extracellular matrix protein 1 isoform precursor, hemopexin precursor, |
| | histidine-rich glycoprotein, dopamine beta-hydroxylase precursor, |
| | peptidoglycan recognition protein L precursor |
| 827 | Chain A, Crystal Structure of Native Heparin Cofactor Ii, fibrinogen gamma |
| | chain, hemopexin precursor |
| 833 | Extracellular matrix protein1 isoform 1 precursor, inter-alpha (globulin) |
| | inhibitor H1, isoform CRA_b, hemopexin precursor |
| 844 | alpha-1-B-glycoprotein, alpha-2-antiplasmin precursor, complement |
| | component 1, vitronectin precursor (unnamed protein), Vitamin K- |
| | dependent protein S precursor, ASPIC |
| 846 | Vitamin K-dependent protein S precursor, |
| | Chain B, Human Complement Component C3, coagulation factor (unnamed |
| | protein), complement 9, GRP78, afamin precursor |

TABLE 6

| | |
|---|---|
| 27 | alpha-2-macroglobulin precursor |
| 76 | fibronectin precursor, Chain B, Structure of complement C3b, C9 |
| | complement protein |
| 93 | no proteins meet the criteria |
| 124 | unnamed protein (coagulation factor II precursor) |
| 206 | alpha-1-antichymotrypsin |
| 210 | alpha-2-macroglobulin precursor, complement component C3, complement |
| | factor H |
| 246 | alpha-2-macroglobulin precursor, complement factor H |
| 255 | macroglobulin alpha2, complement factor H |
| 265 | No proteins meet criteria |
| 292 | alpha-2-macroglobulin precursor, complement factor H, trypsin inhibitor, |
| | inter-alpha-trypsin heavy chain H1 precursor |
| 372 | alpha-2-macroglobulin precursor, complement factor H isoforma precursor, |
| | gelsolin isoform a precursor |
| 384 | Ceruloplasmin, inter-alpha-trypsin inhibitor heavy chain H4 precursor, |
| | phosphatidylinositol-glycan-specific phospholipase D1 precursor |
| 385 | phosphatidylinositol-glycan-specific phospholipase D1 precursor, |
| | ceruloplasmin, inter-alpha-trypsin inhibitor family heavy chain-related |
| | protein |
| 393 | Ceruloplasmin, inter-alpha-trypsin inhibitor heavy chain H4 precursor, |
| | phosphatidylinositol-glycan-specific phospholipase D1 precursor |
| 394 | inter-alpha-trypsin inhibitor heavy chain H4 precursor, ceruloplasmin |
| 408 | glycosylphosphatidylinositol specific phosphatase D1, ceruloplasmin |
| 477 | alpha-2-macroglobulin precursor, complement component C6 precursor |
| | peptide, complement factor B |
| 481 | pre-pro-alpha(I) collagen |
| 495 | Ceruloplasmin, complement component C3, factor H, SERPIN2 protein, gp- |
| | 180-carboxypeptidase D-like enzyme |
| 496 | inter-alpha-trypsin inhibitor family heavy chain-related protein, Chain B, |
| | Human Complement Component C3, alpha-2-macroglobulin precursor, |
| | ceruloplasmin |
| 644 | Fibulin-1 isoform D precursor, inter-alpha-trypsin inhibitor family heavy |
| | chain-related protein, plasminogen, complement factor B, HGF activator, |
| | preproprotein |
| 746 | unnamed protein (coagulation factor II precursor), afamin precursor, |
| | Vitamin K-dependent protein, complement component 1, s subcomponent, |
| | iinter-alpha-trypsin inhibitor, ASPIC |
| 805 | complement component 3 precursor, plasma kallikrein precursor, annexin |
| | A2 isoform 2 |
| 840 | glucosamine (N-acetyl)-6-sulfatase precursor, unnamed protein (cystic |
| | fibrosis antigen), ezrin (p81) (cytovillin) (villin-2), S100 Calcium binding |
| | protein A9 |
| 849 | glucosamine (N-acetyl)-6-sulfatase precursor, coagulation factor XIII A |
| | chain precursor, peptidoglycan recognition protein L precursor, complement |
| | component 4 binding protein |
| 856 | inter-alpha-trypsin inhibitor heavy chain H1 precursor, glucosamine (N- |
| | acetyl)-6-sulfatase precursor, fibrinogen gamma chain, coagulation factor |
| | XIII A chain precursor |
| 864 | glucosamine (N-acetyl)-6-sulfatase precursor, Ig mu chain precursor, |
| | phospholipase D3 isoform, moesin |
| 962 | complement 9, Chain B, Structure of Complement C3b, alpha-2-antiplasmin |
| | precursor, kininogen, thrombin inhibitor |
| 973 | complement 9, Chain A, antithrombin Iii, alpha-2-antiplasmin precursor, |
| | kinninogen, thrombin inhibitor, L-plastin, complement component 1, alpha- |
| | 1-B-glycoprotein, hemopexin precursor |
| 1472 | complement component C4A, complement component C3, apolipoprotein |
| | A-IV precursor, serum paraoxonase/arylesterase, preprohaptoglobulin, |
| | COMP, serpin peptidase inhibitor, clade I, follistatin-like 1 precursor |
| 1498 | complement factor H-related protein 1 precursor, Chain A, Crystal Structure |
| | of Lipid-Free human Apolipoprotein A-1, annexin A2 isoform 2, |
| | phospholipase D3 isoform 2, Chain E, Structure of human transferring |
| | receptor-transferrin complex |
| 44 | fibronectin precursor, complement component C3, alpha-2-macroglobulin precursor |
| 82 | Chain B, Structure of Complement C3b |
| 109 | factor H, fibronectin precursor |
| 126 | fibronectin 1 isoform 3 preproprotein, alpha-2-macroglobulin precursor, |
| 164 | macroglobulin alpha 2 |
| 191 | complement component C3 |
| 205 | no protein matches criteria |
| 216 | collagen type IV alpha 1, alpha 2 type IV collagen preproprotein |
| 244 | collagen type IV alpha 1, alpha 2 type IV collagen preproprotein |
| 252 | alpha 2 macroglobulin, complement factor H, complement component C3 |
| 267 | inter-alpha-trypsin inhibitor, Human Factor H, fibronectin precursor, inter- |
| | alpha-trypsin inhibitor, C-terminal |
| 295 | complement factor H, alpha 2 macroglobulin, trypsin inhibitor, inter-alpha- |
| | trypsin inhibitor |
| 352 | complement factor H, alpha 2 macroglobulin, inter-alpha-trypsin inhibitor, |
| | trypsin inhibitor, complement component C3 |
| 392 | Ceruloplasmin, phosphatidylinositol-glycan-specific phospholipase D1 |
| 396 | afamin precursor, ceruloplasmin, inter-alpha-trypsin inhibitor |
| 469 | complement component 6, isoform CRA_b |
| 509 | inter-alpha-trypsin inhibitor heavy chain-related protein, ceruloplasmin, |
| | Chain B, Structure of Complement C3b |
| 510 | Chain A, Crystal Structure of human Galectin-7 |
| 512 | inter-alpha-trypsin inhibitor family heavy chain-related protein |
| 547 | serum albumin, inter-alpha-trypsin inhibitor family heavy chain-related |
| | protein |
| 533 | COMP, ceruloplasmin |
| 554 | inter-alpha-trypsin inhibitor family heavy chain-related protein |
| 555 | ALB protein, gelsolin isoform a precursor |
| 561 | serum albumin, trypsin inhibitor, complement component C3 |
| 567 | Chain B, Human complement component C3 |
| 568 | complement component C3, complement factor B, gelsolin isoform |
| | precursor |
| 655 | fibulin-1 isoform D precursor, hypothetical protein (inter-alpha-globulin |
| | inhibitor H4), valosin-containing protein, Vitamin D-binding protein |
| | precursor |
| 677 | unnamed protein (complement component 2 precursor), annexin A2, |
| | complement factor B |
| 701 | Annexin A2 isoform 2 |
| 703 | inter-alpha-trypsin inhibitor family heavy chain-related protein |
| 704 | hornerin precursor |
| 712 | complement component 1, s subcomponent, ASPIC, afamin precursor, |
| | Vitamin K-dependent protein |
| 729 | plasma kallikrein precursor, S100 calcium-binding protein A9, Protein |
| | S100-A7 (psoriasin) |
| 744 | gelsolin isoform a precursor |
| 745 | gelsolin isoform a precursor |
| 748 | gelsolin isoform a precursor, coagulation factor XIII, Chain b, Alpha- |
| | Ferrous-Carbonmonoxy |
| 763 | afamin precursor, coagulation factor II precursor, insulin-like growth factor |
| | binding protein, acid labile, histidine-rich glycoprotein precursor, |
| | phospholipid transfer protein isoform a precursor, antithrombin III |
| 764 | glucosamine (N-acetyl)-6-sulfatase precursor, coagulation factor XIII B |
| | chain, gelsolin isoform a precursor |
| 765 | ASPIC, coagulation factor II precursor (unnamed protein), vitronectin |
| | (unnamed protein), histidine-rich glycoprotein precursor, biotinidase |
| | precursor |
| 766 | afamin precursor, insulin-like growth factor binding protein, acid labile |
| | subunit, coagulation factor II precursor, alpha-1-B-glycoprotein, thrombin |
| | inhibitor, C9 complement protein |
| 770 | Coagulation factor XIII B chain precursor |
| 776 | insulin-like growth factor binding protein, hemopexin precursor |
| 818 | vanin 1 precursor, biotinidase precursor, vitronectin (unnamed protein), |
| | ASPIC |
| 825 | extracellular matrix protein 1 isoform precursor, hemopexin precursor, |
| | dopamine beta-hydroxylase precursor, peptidoglycan recognition protein L |
| | precursor |
| 827 | Chain A, Crystal Structure of Native Heparin Cofactor Ii, fibrinogen gamma |
| | chain, hemopexin precursor |
| 833 | Extracellular matrix protein1 isoform 1 precursor, inter-alpha (globulin) |
| | inhibitor H1, isoform CRA_b, hemopexin precursor |
| 844 | alpha-1-B-glycoprotein, alpha-2-antiplasmin precursor, complement |
| | component 1, vitronectin precursor (unnamed protein), Vitamin K- |
| | dependent protein S precursor, ASPIC |
| 846 | Vitamin K-dependent protein S precursor, Chain B, Human Complement |
| | Component C3, coagulation factor (unnamed protein), complement 9, |
| | GRP78, afamin precursor |

1. A method for diagnosing osteoarthritis in a subject, said method comprising steps of:
providing a biological sample obtained from the subject;
determining, in the biological sample, the level of at least one protein selected from the group consisting of proteins identified in Tables 1-6, to obtain a test protein expression profile; and
based on the test protein expression profile obtained, providing an osteoarthritis diagnosis to the subject.

2. The method of claim 1, wherein the protein is selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

3. The method of claim 1, wherein the protein is selected from the group consisting of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, hornerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

4. The method of claim 1, wherein the protein is selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, fibronectin 1 isoform 3 preproprotein, collagen type IV alpha 1, C-terminal inter-alpha trypsin inhibitor, phosphatidylinositol-glycan-specific phospholipase D1, complement component 6 isoform CRA_b, Chain A Crystal Structure of human Galectin-7, ALB protein, valosin-containing protein, Vitamin D-binding protein precursor, complement component 2 precursor, annexin A2, hornerin precursor, Protein S100-A7 (psoriasin), coagulation factor XIII, B Chain Alpha-Ferrous-Carbonmonoxy, insulin-like growth factor binding protein acid labile, histidine-rich glycoprotein precursor, phospholipid transfer protein isoform a precursor, coagulation factor XIII B chain, vitronectin, biotinidase precursor, alpha-1-B-glycoprotein, Coagulation factor XIII B chain precursor, vanin 1 precursor, extracellular matrix protein 1 isoform precursor, histidine-rich glycoprotein, dopamine beta-hydroxylase precursor, Chain A Crystal Structure of Native Heparin Cofactor Ii, inter-alpha (globulin) inhibitor H1, vitronectin precursor, Vitamin K-dependent protein S precursor, GRP78, analogs and fragments thereof; and any combination thereof.

5. The method of claim 1, wherein providing an osteoarthritis diagnosis to the subject comprises steps of:
comparing the test protein expression profile to a control protein expression profile, wherein a difference between the test protein expression profile and the control protein expression profile is indicative of a stage of osteoarthritis; and based on the comparison, identifying osteoarthritis suffered by the subject as early stage osteoarthritis or late stage osteoarthritis.

6. The method of claim 5, wherein the control protein expression profile is an early osteoarthritis expression profile, and the difference is indicative of late stage osteoarthritis.

7. The method claim 6, wherein the difference is selected from the group consisting of: an increase in the level of expression of one or more proteins selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, fibronectin 1 isoform 3 preproprotein, collagen type IV alpha 1, C-terminal inter-alpha trypsin inhibitor, phosphatidylinositol-glycan-specific phospholipase D1, complement component 6 isoform CRA_b, Chain A Crystal Structure of human Galectin-7, ALB protein, valosin-containing protein, Vitamin D-binding protein precursor, complement component 2 precursor, annexin A2, hornerin precursor, Protein S100-A7 (psoriasin), coagulation factor XIII, B Chain Alpha-Ferrous-Carbonmonoxy, insulin-like growth factor binding protein acid labile, histidine-rich glycoprotein precursor, phospholipid transfer protein isoform a precursor, coagulation factor XIII B chain, vitronectin, biotinidase precursor, alpha-1-B-glycoprotein, Coagulation factor XIII B chain precursor, vanin 1 precursor, extracellular matrix protein 1 isoform precursor, histidine-rich glycoprotein, dopamine beta-hydroxylase precursor, Chain A Crystal Structure of Native Heparin Cofactor Ii, inter-alpha (globulin) inhibitor H1, vitronectin precursor, Vitamin K-dependent protein S precursor, GRP78, analogs and fragments thereof; and any combination thereof.

8. The method of claim 5, wherein the protein is selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

9. The method of claim 8, wherein the biological sample comprises a sample of synovial fluid.

10. The method of claim 1, wherein the subject is a human being.

11. The method of claim 10, wherein the subject is suspected of having osteoarthritis.

12. The method of claim 1 further comprising a step of: selecting a therapy for the subject based on the osteoarthritis diagnosis.

13. An osteoarthritis (OA) expression profile map comprising expression level information for one or more of polypeptides selected from the group consisting of alpha-2-macroglobulin precursor, fibronectin precursor, Chain B structure of complement C3b, C9 complement protein, coagulation factor II precursor, alpha-1-antichymotrypsin, complement factor H, inter-alpha-trypsin heavy chain H1 precursor, complement factor H isoform a precursor, gelsolin isoform a precursor, inter-alpha-trypsin inhibitor heavy chain H4 precursor, phosphatidylinositol-glycan-specific phospholipase D1 precursor, inter-alpha-trypsin inhibitor family heavy chain-related protein, glycosylphosphatidylinositol specific phosphatase D1, complement component C6 precursor peptide, complement factor B, pre-pro-alpha(I) collagen, SERPIN2 protein, gp-180-carboxypeptidase D-like enzyme, Fibulin-1 isoform D precursor, plasminogen, HGF activator preproprotein, afamin precursor, Vitamin K-dependent protein, complement component 1 s subcomponent, inter-alpha-trypsin inhibitor, ASPIC, complement component 3 precursor, plasma kallikrein precursor, annexin A2 isoform 2, glucosamine(N-acetyl)-6-sulfatase precursor, (cystic fibrosis antigen), ezrin (p81) (cytovillin) (villin-2), S100 Calcium binding protein A9, coagulation factor XIII A chain precursor, peptidoglycan recognition protein L precursor, complement component 4 binding protein, inter-alpha-trypsin inhibitor heavy chain H1 precursor, fibrinogen gamma chain, Ig mu chain precursor, phospholipase D3 isoform, moesin, alpha-2-antiplasmin precursor, kininogen, thrombin inhibitor, Chain A antithrombin III, L-plastin, complement component 1, alpha-1-B-glycoprotein, hemopexin precursor, complement component C4A, apolipoprotein A-IV precursor, serum paraoxonase/arylesterase, preprohaptoglobulin, COMP, serpin peptidase inhibitor clade I, follistatin-like 1 precursor, complement factor H-related protein 1 precursor, Chain A Crystal Structure of Lipid-Free human Apolipoprotein A-1, phospholipase D3 isoform 2, Chain E Structure of human transferring receptor-transferrin complex, complement component C3, analogs and fragments thereof; and any combination thereof.

14. The OA expression profile map of claim 13, wherein the expression profile map comprises expression level information for at least one biological sample obtained from a healthy individual, an individual with early stage osteoarthritis or an individual with late stage osteoarthritis.

15. An OA expression profile map comprising expression level information for one or more of proteins selected from the group consisting of fibronectin precursor, alpha-2-macroglobulin precursor, Chain B Structure of Complement C3b, complement factor H, fibronectin 1 isoform 3 preproprotein, collagen type IV alpha 1, alph 2 type IV collagen preprotein, inter-alpha-trypsin inhibitor, C-terminal inter-alpha trypsin inhibitor, phosphatidylinositol-glycan-specific phospholipase D1, afamin precursor, complement component 6 isoform CRA_b, inter-alpha-trypsin inhibitor heavy chain-related protein, Chain A Crystal Structure of human Galectin-7, COMP, ALB protein, gelsolin isoform a precursor, complement factor B, fibulin-1 isoform D precursor, valosin-containing protein, Vitamin D-binding protein precursor, complement component 2 precursor, annexin A2, Annexin A2 isoform 2, hornerin precursor, complement component 1 s subcomponent, ASPIC, Vitamin K-dependent protein, plasma kallikrein precursor, S100 calcium-binding protein A9, Protein S100-A7 (psoriasin), coagulation factor XIII, B Chain Alpha-Ferrous-Carbonmonoxy, coagulation factor II precursor, insulin-like growth factor binding protein acid labile subunit, histidine-rich glycoprotein precursor, phospholipid transfer protein isoform a precursor, antithrombin III, glucosamine(N-acetyl)-6-sulfatase precursor, coagulation factor XIII B chain, vitronectin, biotinidase precursor, acid labile subunit, alpha-1-B-glycoprotein, thrombin inhibitor, C9 complement protein, Coagulation factor XIII B chain precursor, hemopexin precursor, vanin 1 precursor, extracellular matrix protein 1 isoform precursor, histidine-rich glycoprotein, dopamine beta-hydroxylase precursor, peptidoglycan recognition protein L precursor, Chain A Crystal Structure of Native Heparin Cofactor Ii, fibrinogen gamma chain, inter-alpha (globulin) inhibitor H1, alpha-2-antiplasmin precursor, vitronectin precursor, Vitamin K-dependent protein S precursor, complement component 9, GRP78, analogs and fragments thereof; and any combination thereof.

16. The OA expression profile map of claim 15, wherein the expression profile map comprises expression level information for at least one biological sample obtained from a healthy individual, an individual with early stage osteoarthritis or an individual with late stage osteoarthritis.

17-20. (canceled)
